(19) United States
(12) Patent Application Publication
(10) Pub. No.: US 2024/0425598 A1
(43) Pub. Date: Dec. 26, 2024

(54) RECEPTOR-MEDIATED DELIVERY OF NUCLEIC ACIDS

(71) Applicants: KODIKAZ THERAPEUTIC SOLUTIONS, INC., New York, NY (US); EMORY UNIVERSITY, Atlanta, GA (US)

(72) Inventors: Anthony JOHNSON, Long Island City, NY (US); Leon BERNAL-MIZRACHI, Atlanta, GA (US); Munevver CINAR, Brookhaven, GA (US)

(21) Appl. No.: 18/735,738

(22) Filed: Jun. 6, 2024

Related U.S. Application Data
(63) Continuation of application No. PCT/US22/81039, filed on Dec. 6, 2022.
(60) Provisional application No. 63/286,915, filed on Dec. 7, 2021.
(60) Provisional application No. 63/369,908, filed on Jul. 29, 2022.

(51) Int. Cl. C07K 16/28; C07K 16/44

(52) U.S. Cl. CPC C07K 16/2833; C07K 16/44; C07K 2317/76

(57) ABSTRACT

Methods disclosed herein relate to receptor-mediated uptake of circulating tumor DNA (ctDNA) and derivatives thereof, including synthetic delivery systems. Provided are methods of identifying a receptor that mediates or is associated with uptake of ctDNA, methods of reducing ctDNA uptake by target cells, methods of determining an expression level of a receptor associated with uptake of ctDNA, and methods of treatment that can be based on an expression level of the receptor.

### CROSS REFERENCE

This application is a continuation of International Application No. PCT/US22/81039, filed Dec. 6, 2022, which claims the benefit of U.S. Provisional Patent Application No. 63/369,908, filed Jul. 29, 2022, and U.S. Provisional Patent Application No. 63/286,915, filed Dec. 7, 2021, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jun. 5, 2024, is named 54774_706_301_SL.txt and is 1,032,708 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Tumor-derived cell-free DNA can contain genetic alterations relevant to tumorigenesis, suggesting circulating tumor-derived DNA (ctDNA) serves as a vehicle for genetic exchange between tumor cells. It has been suggested that ctDNA can transfer oncogenic gene mutations for reshaping the tumor microenvironment. However, the underlying process and mechanisms remain poorly understood.

### BRIEF DESCRIPTION OF THE DRAWINGS

FIG. 1A shows the percent of MMI1s multiple myeloma cells, MIA pancreatic cancer cells, and HCT116 colon cancer cells that were positive for ctDNA signal after incubation with CY5 labelled-ctDNA from multiple myeloma patients (A, D, E), pancreatic cancer patients (G, I, F), or colon cancer patients (KLM), with (+T) or without (−T) trypsin treatment to cleave cell surface-exposed membrane proteins.

FIG. 1B shows representative flow cytometry scatterplots of MMIs multiple myeloma cells, MIA pancreatic cancer cells, and HCT116 colon cancer cells after incubation with CY5 labelled-ctDNA from a patient with a corresponding type of cancer, with or without trypsin treatment to cleave cell surface-exposed membrane proteins.

FIG. 2 shows the effect of pre-incubating ctDNA from multiple myeloma (MM) or pancreatic cancer (PC) patients with anti-dsDNA antibodies (SLE1, SLE2) or IgG isotype control antibodies (IgG) on nuclear localization of ctDNA. CY5-labelled ctDNA was incubated with 1 g/mL of anti-dsDNA antibodies for 24 hours, then added to cultures of cell lines of the same cell type as the ctDNA's tissue of origin (MMIs—multiple myeloma; MIA—pancreatic cancer). 24 hours later, samples were imaged and the nuclear intensity of ctDNA determined.

FIG. 3 outlines an experiment to identify proteins that can function as ctDNA recognizing receptors.

FIG. 4 provides illustrative membrane proteins identified as candidates that bind to ctDNA by a pull down and mass spectrometry assay.

FIG. 5 shows confocal fluorescence microscopy images of PANC-1 pancreatic cancer cells that were pre-incubated with anti-HLA antibody (right panel) or IgG isotype control antibody (left panel), then treated with rhodamine labelled ctDNA from a pancreatic cancer patient.

FIG. 6A and FIG. 6B show representative fluorescence microcopy images of IMIs cells treated with rhodamine-labelled ctDNA with or without pre-treatment with anti-HLA-A antibody or an IgG isotype control.

FIG. 6C shows the average nuclear intensity of ctDNA (rhodamine-labelled) for HLA positive cells (left) or HLA negative IMIs cells (right) that were pre-treated for 4 hours with titrating doses of anti-HLA-A antibody or IgG isotype control (doses: 0, 0.25, 0.5, or 1 μg/mL), after which rhodamine-labeled ctDNA was added to the culture wells at 1 μg/mL for 24 hours. Cells were fixed and stained with a fluorescent secondary antibody to stain HLA-A-expressing cells and differentiate them from cells that were HLA-A negative.

FIG. 7 shows the level of rhodamine-labelled multiple myeloma ctDNA in the nuclei of MMIs cells treated with antibodies against CD41, CD97, Integrin αVβ5 (Int-P1F6), P142A, TMEM, or PTK7A. Data are shown for cells that stained positive for the anti-receptor antibodies after labelling with an AF488-conjugated secondary antibody.

FIG. 8A shows the results of an Amplified Luminescent Proximity Homogeneous Assay (ALPHA) for ctDNA-receptor binding performed on multiple myeloma cell lines (e.g., MM1s, OPM1, RPMI, JK6L) with multiple myeloma ctDNA. Increases in the IgG/biotin ratio of more than threefold were considered indicative of a receptor that bound to multiple myeloma ctDNA.

FIG. 8B shows the results of an Amplified Luminescent Proximity Homogeneous Assay (ALPHA) for ctDNA-receptor binding performed on multiple myeloma cell lines (e.g., MM1s, OPM1, RPMI, JK6L) and non-myeloma cell lines (e.g., MIA, HTC116, A549, MDA-MD-468) with multiple myeloma ctDNA. Increases in the IgG/biotin ratio of more than threefold were considered indicative of a receptor that bound to multiple myeloma ctDNA.

FIG. 9 shows competitive inhibition of labeled ctDNA or synthetic delivery system polynucleotide uptake by co-treatment with unlabeled ctDNA or synthetic delivery system polynucleotide.

FIG. 10 shows the results of a fluorescence microscopy assay demonstrating colocalization of ctDNA with APLP2, PKP4, or HLA-A at the cell membrane.

### SUMMARY

Disclosed herein, in some aspects, is a method of reducing uptake of circulating tumor-derived DNA (ctDNA) by target cells in a subject, the method comprising administering to the subject an antagonist of a receptor that is associated with cellular uptake of the ctDNA, thereby reducing the uptake of the ctDNA by the target cells in the subject.

Disclosed herein, in some aspects, is a method of reducing uptake of circulating tumor-derived DNA (ctDNA) by target cells, the method comprising contacting the target cells with an antagonist of a receptor that is associated with uptake of the ctDNA, thereby reducing the uptake of the ctDNA by the target cells.

Disclosed herein, in some aspects, is a method comprising determining that a biological sample from a subject expresses a receptor that is associated with uptake of ctDNA at a level that is associated with the uptake of the ctDNA.

Disclosed herein, in some aspects, is a method of treating a condition in a subject in need thereof, the method comprising administering a therapeutically-effective amount of a therapeutic agent to the subject, wherein the administering is based at least in part on a result of an assay performed on a biological sample from the subject, wherein the assay determined that a receptor associated with uptake of ctDNA is expressed in the biological sample at a level that is associated with the uptake of the ctDNA.

Disclosed herein, in some aspects, is a method of identifying a receptor that mediates uptake of a cargo by a target cell, the method comprising: (a) isolating ctDNA or a derivative thereof that is bound to the receptor or a fragment thereof; and (b) identifying the receptor.

Disclosed herein, in some aspects, is a method of delivering a cargo to a target cell, the method comprising contacting a receptor on the target cell with a synthetic delivery system that comprises a polynucleotide, thereby inducing uptake of the cargo by the target cell, wherein: (a) the polynucleotide comprises a first nucleic acid sequence identified in circulating tumor DNA (ctDNA) from a substantially similar cell type as the target cell; and (b) the receptor comprises an armadillo-like protein, an APP (amyloid precursor protein) family member, a p120(ctn)/plakophilin subfamily member, an NMC protein, a GPCR, an adhesion protein, a tyrosine phosphatase, an integrin, an ion channel, a mineral transporter, a nutrient transporter, or an ATPase.

### DETAILED DESCRIPTION

Compositions and methods provided herein relate to receptor-mediated cellular uptake of nucleic acids, for example, uptake of circulating tumor DNA/circulating tumor-derived DNA (ctDNA) and synthetic delivery systems derived therefrom.

ctDNA can target and transmit genetic material to target cells that resemble the ctDNA's cell of origin. For example, ctDNA can be internalized by target cells, transported to the nucleus, and integrated into the genome of the target cells. The transfer of ctDNA to target cells has been associated with, for example, induction or transfer of resistance or sensitivity of a cancer to anti-cancer agents, such as chemotherapeutic drugs.

Disclosed herein are receptors that mediate binding to and/or internalization of ctDNA by target cells, and related compositions and methods.

Antagonists of receptors disclosed herein can be used to reduce uptake of ctDNA by target cells. Blocking uptake of ctDNA by target cells (e.g., cancer cells, or cells of a similar cell type as the cancer cells) can be useful, for example, for delaying or reducing resistance of cancer cells to anti-cancer therapeutic agents, such as chemotherapeutic drugs. An antagonist (e.g., an antibody or antigen-binding fragment thereof) can be contacted to a target cell and can bind to the receptor on the target cell, thereby reducing uptake of ctDNA by the target cell. An antagonist can be administered to a subject to inhibit cancer progression, cancer metastasis, or resistance of the cancer to anti-cancer therapeutic agents, for example.

Receptors disclosed herein can be targeted to induce uptake of a ctDNA or synthetic delivery system. Synthetic delivery systems disclosed herein can utilize polynucleotides to facilitate binding, uptake, nuclear trafficking, and/or genomic integration of a component of the synthetic delivery system, for example, a cargo. The synthetic delivery system can be used for delivery of a cargo to cells that express the receptor, including cancer cells and/or specific cell types.

A therapeutic agent, for example, comprising a synthetic delivery system, can be administered to a subject on the basis of whether target cells in the subject express a receptor associated with uptake of ctDNA at a level that is associated with uptake of the ctDNA. For example, a biological sample (e.g., blood sample, cancer cell sample, or tissue (e.g., cancer) biopsy) from a subject can be assayed for expression of the receptor, and a therapeutic agent comprising a synthetic delivery system can be administered to the subject if the corresponding receptor for the synthetic delivery system is detected as expressed at least at a suitable level.

Also disclosed herein are methods of identifying receptors that mediate uptake of ctDNA or a derivative thereof by a target cell, and various related assays, for example, assays for confirming binding of a candidate receptor to ctDNA or a synthetic delivery system derived therefrom, assays for evaluating a role of the receptor in uptake of the ctDNA or synthetic delivery system, assays for determining expression levels of the receptor in biological samples, and assays for determining whether the receptor is expressed at a level that is associated with uptake of the ctDNA.

Methods disclosed herein can also comprise delivering a cargo to a target cell. For example, a target cell can comprise a receptor, and uptake of the cargo by the target cell can be induced by contacting the receptor on the target cell with a synthetic delivery system and/or polynucleotide disclosed herein.

### Receptors

Compositions and methods disclosed herein can utilize receptors. Methods of the disclosure can comprise, for example, reducing uptake of ctDNA by contacting a target cell with an antagonist of the receptor, delivering a cargo to a target cell by contacting a receptor with a synthetic delivery system or a polynucleotide that binds to the receptor, assaying a level of a receptor expressed in a biological sample, and/or administering an agent that binds to and/or induces uptake via a receptor based on an expression level of the receptor in the biological sample. Methods disclosed herein can comprise identifying a receptor that can be used to deliver a cargo to a target cell.

A receptor can be expressed by a target cell disclosed herein. In some embodiments, expression of the receptor is specific to a target cell, target cell type, or target cell population, thereby facilitating specific delivery of a cargo to the target cell, target cell type, or target cell population. In some embodiments, expression of the receptor is relatively higher on a target cell, target cell type, or target cell population than a control cell, control cell type, or control cell population, thereby facilitating delivery of a cargo to the target cell, target cell type, or target cell population.

In some embodiments, expression of the receptor is not specific to a target cell, target cell type, or target cell population. In some embodiments, expression of the receptor is not relatively higher on a target cell, target cell type, or target cell population than a control cell, control cell type, or control cell population. In some embodiments, delivery of a cargo to a target cell, target cell type, or target cell population is higher than delivery to a control cell, control cell type, or control cell population even though expression of the receptor is not higher or is substantially similar on the target cell, target cell type, or target cell population than the control cell, control cell type, or control cell population. For example, the receptor can exhibit a different activity despite similar expression, or additional receptors, adaptor proteins, or cooperative factors can contribute to uptake of the cargo by the target cells in combination with the receptor.

Binding of a synthetic delivery system or a polynucleotide to a receptor disclosed herein can induce uptake of the synthetic delivery system, polynucleotide, or cargo associated therewith by, for example, endocytosis, receptor-mediated endocytosis, clathrin-dependent endocytosis, caveolae-dependent endocytosis, CLIC/GEEC pathway uptake, lipid-raft associated endocytosis, phagocytosis, macropinocytosis, micropinocytosis, pinocytosis, or endosomal recycling, e.g., of MHC, such as HLA-A.

A receptor can be or can comprise a MHC protein or a component thereof. A receptor can be or can comprise a human leukocyte antigen or a component thereof. A receptor can be or can comprise a class I MHC protein or a component thereof. For example, a receptor can be or can comprise HLA-A or a component thereof. A receptor can be or can comprise a protein that interacts with MHC class I molecules, for example, APLP2.

A receptor can be or can comprise a G-protein coupled receptor (GPCR) or a component thereof. The GPCR can be a class A (rhodopsin-like), class B (e.g., secretin and adhesion subfamilies), class C (glutamate), or class F (Frizzled) family or subfamily GPCR. A GPCR can be a GPCR that elicits a cyclic adenosine 3,5-monophosphate (cAMP) response, calcium mobilization, or phosphorylation of extracellular regulated protein kinases 1/2 (pERK1/2). For example, a receptor can be or can comprise CD97 or a component thereof.

A receptor can be or can comprise an adhesion protein or a component thereof. For example, a receptor can be or can comprise CD97 or a component thereof, PTK7 or a component thereof, ITGB5 or a component thereof, ITGA2B or a component thereof, or αVβ5 integrin or a component thereof.

A receptor can be or can comprise an integrin or a component thereof. A receptor can be or can comprise, for example, ITGB5 or a component thereof, ITGA2B or a component thereof, or αVβ5 integrin or a component thereof.

A receptor can be or can comprise a tyrosine phosphatase or a component thereof. A receptor can be or can comprise a protein tyrosine phosphatase or a component thereof. A receptor can be or can comprise a protein tyrosine phosphatase receptor or a component thereof. A receptor can be or can comprise, for example, PTPRF or a component thereof.

A receptor can be or can comprise an ion channel or a component thereof. A receptor can comprise a sensory neuron ion channel, such as a nociceptor ion channel. A receptor can comprise a function in mechanosensation. A receptor can be or can comprise, for example, TMEM120A or a component thereof.

A receptor can be or can comprise a mineral transporter or a component thereof. A receptor can be or can comprise a ferroxidase or a component thereof. A receptor can be or can comprise, for example, ceruloplasmin or a component thereof.

A receptor can be or can comprise a metalloprotein or a component thereof. A receptor can be or can comprise, for example, ceruloplasmin or a component thereof.

A receptor can be or can comprise a nutrient transporter or a component thereof. A receptor can be or can comprise a monocarboxylate transporter or a component thereof. A receptor can be or can comprise, for example, SLC16A1 or a component thereof. A receptor can be or can comprise an amino acid transporter or a component thereof. A receptor can be or can comprise, for example, SLC7A5 or a component thereof.

A receptor can be or can comprise a receptor of fibronectin, fibrinogen, plasminogen, prothrombin, thrombospondin, vitronectin, or a combination thereof. For example, a receptor can be or can comprise ITGA2B or a component thereof.

A receptor can be or can comprise an ATPase or a component thereof. A receptor can be or can comprise a sodium/potassium transporting ATPase or a component thereof. A receptor can be or can comprise, for example, ATP1B1 or a component thereof. A receptor can be or can comprise a calcium transporter or a component thereof. A receptor can be or can comprise an ion transporting ATPase or a component thereof. A receptor can be, for example, ATP2B4 or a component thereof.

A receptor can be or can comprise an Armadillo-like protein, for example, comprising one or more armadillo repeats. A receptor can be or can comprise a member of a p120(ctn)/plakophilin subfamily of Armadillo-like proteins, including for example CTNND1, CTNND2, PKP1, PKP2, PKP4, or ARVCF. In some embodiments a receptor is PKP4.

A receptor can be or can comprise an APP (amyloid precursor protein) family member, for example, APP, APLP1, or APLP2. In some embodiments, a receptor is APLP2.

A receptor can be or can comprise a kinase. A receptor can be or can comprise a lipid kinase. A receptor can be or can comprise a Phosphatidylinositol kinase. A receptor can be or can comprise a phosphatidylinositol-5-phosphate 4-kinase family member. A receptor can be capable of catalyzing the phosphorylation of phosphatidylinositol-5-phosphate on the fourth hydroxyl of the myo-inositol ring to form phosphatidylinositol-5,4-bisphosphate. A receptor can be or can comprise PIP4K2A.

A receptor can be or can comprise P142A or a component thereof.

In some embodiments, a particular cargo or type of cargo disclosed herein can be delivered by targeting a synthetic delivery system to a receptor or type of receptor disclosed herein, for example, targeting via a polynucleotide and/or recognition sequence.

In some embodiments, targeting a synthetic delivery system to a receptor or type of receptor disclosed herein can increase specific binding, uptake, nuclear localization, and/or genomic integration of the synthetic delivery system, polynucleotide, and/or cargo by target cells relative to control cells.

In some embodiments, a receptor comprises, consists essentially of, or consists of a single protein, receptor, or subunit disclosed herein, for example, APLP2, PKP4, HLA-A, CD41, CD97, ITGB5, ITGA2B, Integrin αVβ5, PI4K2A, TMEM120A, PTK7A, Ceruloplasmin, PTPRF, SLC16A1, SLC7A5, ATP1B1, ATP2B4, P142A, or PIP4K2A.

In some embodiments, the receptor comprises, consists essentially of, or consists of APLP2. In some embodiments, the receptor comprises, consists essentially of, or consists of PKP4. In some embodiments, the receptor comprises, consists essentially of, or consists of HLA-A. In some embodiments, the receptor comprises, consists essentially of, or consists of CD41. In some embodiments, the receptor comprises, consists essentially of, or consists of CD97. In some embodiments, the receptor comprises, consists essentially of, or consists of Integrin αVβ5. In some embodiments, the receptor comprises, consists essentially of, or consists of PI4K2A. In some embodiments, the receptor comprises, consists essentially of, or consists of TMEM120A. In some embodiments, the receptor comprises, consists essentially of, or consists of PTK7A. In some embodiments, the receptor comprises, consists essentially of, or consists of Ceruloplasmin. In some embodiments, the receptor comprises, consists essentially of, or consists of PTPRF. In some embodiments, the receptor comprises, consists essentially of, or consists of SLC16A1. In some embodiments, the receptor comprises, consists essentially of, or consists of SLC7A5. In some embodiments, the receptor comprises, consists essentially of, or consists of ATP1B1. In some embodiments, the receptor comprises, consists essentially of, or consists of ATP2B4. In some embodiments, the receptor comprises, consists essentially of, or consists of P142A. In some embodiments, the receptor comprises, consists essentially of, or consists of PIP4K2A.

In some embodiments, a receptor comprises, consists essentially of, or consists of two proteins or subunits disclosed herein, or two receptors, proteins, or subunits contribute to uptake, for example, two of APLP2, PKP4, HLA-A, CD41, CD97, Integrin αVβ5, PI4K2A, TMEM120A, PTK7A, Ceruloplasmin, PTPRF, SLC16A1, SLC7A5, ATP1B1, ATP2B4, P142A, and PIP4K2A. In some embodiments, a receptor comprises, consists essentially of, or consists of APLP2 and PKP4. In some embodiments, a receptor comprises, consists essentially of, or consists of APLP2 and HLA-A. In some embodiments, a receptor comprises, consists essentially of, or consists of PKP4 and HLA-A. In some embodiments, a receptor comprises, consists essentially of, or consists of ITGB5 and ITGA2B, e.g., Integrin αVβ5.

In some embodiments, a receptor comprises, consists essentially of, or consists of three proteins or subunits disclosed herein, or three receptors, proteins, or subunits contribute to uptake, for example for example, three of APLP2, PKP4, HLA-A, CD41, CD97, ITGB5, ITGA2B, Integrin αVβ5, PI4K2A, TMEM120A, PTK7A, Ceruloplasmin, PTPRF, SLC16A1, SLC7A5, ATP1B1, ATP2B4, P142A, and PIP4K2A.

In some embodiments, a receptor comprises, consists essentially of, or consists of four proteins or subunits disclosed herein, or four receptors, proteins, or subunits contribute to uptake, for example, for example, four of APLP2, PKP4, HLA-A, CD41, CD97, ITGB5, ITGA2B, Integrin αVβ5, PI4K2A, TMEM120A, PTK7A, Ceruloplasmin, PTPRF, SLC16A1, SLC7A5, ATP1B1, ATP2B4, P142A, and PIP4K2A.

In some embodiments, a single protein or receptor disclosed herein mediates binding and/or internalization of a ctDNA, derivative thereof, synthetic delivery system, or polynucleotide disclosed herein, for example, APLP2, PKP4, HLA-A, CD41, CD97, ITGB5, ITGA2B, Integrin αVβ5, PI4K2A, TMEM120A, PTK7A, Ceruloplasmin, PTPRF, SLC16A1, SLC7A5, ATP1B1, ATP2B4, PIP4K2A, or P142A.

In some embodiments, APLP2 mediates binding and/or internalization of the ctDNA, derivative thereof, synthetic delivery system, or polynucleotide. In some embodiments, PKP4 mediates binding and/or internalization of the ctDNA, derivative thereof, synthetic delivery system, or polynucleotide. In some embodiments, HLA-A mediates binding and/or internalization of the ctDNA, derivative thereof, synthetic delivery system, or polynucleotide. In some embodiments, APLP2 mediates binding and/or internalization of the ctDNA, derivative thereof, synthetic delivery system, or polynucleotide. In some embodiments, PKP4 mediates binding and/or internalization of the ctDNA, derivative thereof, synthetic delivery system, or polynucleotide. In some embodiments, HLA-A mediates binding and/or internalization of the ctDNA, derivative thereof, synthetic delivery system, or polynucleotide. In some embodiments, CD41 mediates binding and/or internalization of the ctDNA, derivative thereof, synthetic delivery system, or polynucleotide. In some embodiments, CD97 mediates binding and/or internalization of the ctDNA, derivative thereof, synthetic delivery system, or polynucleotide. In some embodiments, Integrin αVβ5 mediates binding and/or internalization of the ctDNA, derivative thereof, synthetic delivery system, or polynucleotide. In some embodiments, PI4K2A mediates binding and/or internalization of the ctDNA, derivative thereof, synthetic delivery system, or polynucleotide. In some embodiments, TMEM120A mediates binding and/or internalization of the ctDNA, derivative thereof, synthetic delivery system, or polynucleotide. In some embodiments, PTK7A mediates binding and/or internalization of the ctDNA, derivative thereof, synthetic delivery system, or polynucleotide. In some embodiments, Ceruloplasmin mediates binding and/or internalization of the ctDNA, derivative thereof, synthetic delivery system, or polynucleotide. In some embodiments, PTPRF mediates binding and/or internalization of the ctDNA, derivative thereof, synthetic delivery system, or polynucleotide. In some embodiments, SLC16A1 mediates binding and/or internalization of the ctDNA, derivative thereof, synthetic delivery system, or polynucleotide. In some embodiments, SLC7A5 mediates binding and/or internalization of the ctDNA, derivative thereof, synthetic delivery system, or polynucleotide. In some embodiments, ATP1B1 mediates binding and/or internalization of the ctDNA, derivative thereof, synthetic delivery system, or polynucleotide. In some embodiments, ATP2B4 mediates binding and/or internalization of the ctDNA, derivative thereof, synthetic delivery system, or polynucleotide. In some embodiments, PIP4K2A mediates binding and/or internalization of the ctDNA, derivative thereof, synthetic delivery system, or polynucleotide. In some embodiments, P142A mediates binding and/or internalization of the ctDNA, derivative thereof, synthetic delivery system, or polynucleotide.

In some embodiments, one or more proteins, subunits, or receptors disclosed herein mediate binding and/or internalization of a ctDNA, derivative thereof, synthetic delivery system, or polynucleotide disclosed herein, for example, one or more of APLP2, PKP4, HLA-A, CD41, CD97, ITGB5, ITGA2B, Integrin αVβ5, PI4K2A, TMEM120A, PTK7A, Ceruloplasmin, PTPRF, SLC16A1, SLC7A5, ATP1B1, ATP2B4, PIP4K2A, and P142A.

In some embodiments, APLP2 and one or more additional proteins mediate binding and/or internalization of the ctDNA, derivative thereof, synthetic delivery system, or polynucleotide. In some embodiments, PKP4 and one or more additional proteins mediate binding and/or internalization of the ctDNA, derivative thereof, synthetic delivery system, or polynucleotide. In some embodiments, HLA-A and one or more additional proteins mediate binding and/or internalization of the ctDNA, derivative thereof, synthetic delivery system, or polynucleotide. In some embodiments, CD41 and one or more additional proteins mediate binding and/or internalization of the ctDNA, derivative thereof, synthetic delivery system, or polynucleotide. In some embodiments, CD97 and one or more additional proteins mediate binding and/or internalization of the ctDNA, derivative thereof, synthetic delivery system, or polynucleotide. In some embodiments, Integrin αVβ5 and one or more additional proteins mediate binding and/or internalization of the ctDNA, derivative thereof, synthetic delivery system, or polynucleotide. In some embodiments, PI4K2A and one or more additional proteins mediate binding and/or internalization of the ctDNA, derivative thereof, synthetic delivery system, or polynucleotide. In some embodiments, TMEM120A and one or more additional proteins mediate binding and/or internalization of the ctDNA, derivative thereof, synthetic delivery system, or polynucleotide. In some embodiments, PTK7A and one or more additional proteins mediate binding and/or internalization of the ctDNA, derivative thereof, synthetic delivery system, or polynucleotide. In some embodiments, Ceruloplasmin and one or more additional proteins mediate binding and/or internalization of the ctDNA, derivative thereof, synthetic delivery system, or polynucleotide. In some embodiments, PTPRF and one or more additional proteins mediate binding and/or internalization of the ctDNA, derivative thereof, synthetic delivery system, or polynucleotide. In some embodiments, SLC16A1 and one or more additional proteins mediate binding and/or internalization of the ctDNA, derivative thereof, synthetic delivery system, or polynucleotide. In some embodiments, SLC7A5 and one or more additional proteins mediate binding and/or internalization of the ctDNA, derivative thereof, synthetic delivery system, or polynucleotide. In some embodiments, ATP1B1 and one or more additional proteins mediate binding and/or internalization of the ctDNA, derivative thereof, synthetic delivery system, or polynucleotide. In some embodiments, ATP2B4 and one or more additional proteins mediate binding and/or internalization of the ctDNA, derivative thereof, synthetic delivery system, or polynucleotide. In some embodiments, PIP4K2A and one or more additional proteins mediate binding and/or internalization of the ctDNA, derivative thereof, synthetic delivery system, or polynucleotide. In some embodiments, P142A and one or more additional proteins mediate binding and/or internalization of the ctDNA, derivative thereof, synthetic delivery system, or polynucleotide.

In some embodiments, two proteins, receptors, or subunits disclosed herein mediate binding and/or internalization of a ctDNA, derivative thereof, synthetic delivery system, or polynucleotide disclosed herein, for example, two of APLP2, PKP4, HLA-A, CD41, CD97, ITGB5, ITGA2B, Integrin αVβ5, PI4K2A, TMEM120A, PTK7A, Ceruloplasmin, PTPRF, SLC16A1, SLC7A5, ATP1B1, ATP2B4, PIP4K2A, and P142A. In some embodiments APLP2 and PKP4 mediate binding and/or internalization of the ctDNA, derivative thereof, synthetic delivery system, or polynucleotide. In some embodiments, APLP2 and HLA-A mediate binding and/or internalization of the ctDNA, derivative thereof, synthetic delivery system, or polynucleotide. In some embodiments, PKP4 and HLA-A mediate binding and/or internalization of the ctDNA, derivative thereof, synthetic delivery system, or polynucleotide.

In some embodiments, two or more proteins, receptors, or subunits disclosed herein mediate binding and/or internalization of a ctDNA, derivative thereof, synthetic delivery system, or polynucleotide disclosed herein, for example, two or more of APLP2, PKP4, HLA-A, CD41, CD97, ITGB5, ITGA2B, Integrin αVβ5, PI4K2A, TMEM120A, PTK7A, Ceruloplasmin, PTPRF, SLC16A1, SLC7A5, ATP1B1, ATP2B4, PIP4K2A, and P142A. In some embodiments, APLP2, PKP4, and one or more additional proteins mediate binding and/or internalization of the ctDNA, derivative thereof, synthetic delivery system, or polynucleotide. In some embodiments, APLP2, HLA-A, and one or more additional proteins mediate binding and/or internalization of the ctDNA, derivative thereof, synthetic delivery system, or polynucleotide. In some embodiments, PKP4, HLA-A, and one or more additional proteins mediate binding and/or internalization of the ctDNA, derivative thereof, synthetic delivery system, or polynucleotide.

In some embodiments, three proteins, receptors, or subunits disclosed herein mediate binding and/or internalization of a ctDNA, derivative thereof, synthetic delivery system, or polynucleotide disclosed herein, for example, three of APLP2, PKP4, HLA-A, CD41, CD97, ITGB5, ITGA2B, Integrin αVβ5, PI4K2A, TMEM120A, PTK7A, Ceruloplasmin, PTPRF, SLC16A1, SLC7A5, ATP1B1, ATP2B4, PIP4K2A, and P142A. In some embodiments, APLP2, PKP4, and HLA-A mediate binding and/or internalization of the ctDNA, derivative thereof, synthetic delivery system, or polynucleotide.

In some embodiments, three or more proteins, receptors, or subunits disclosed herein mediate binding and/or internalization of a ctDNA, derivative thereof, synthetic delivery system, or polynucleotide disclosed herein, for example, three or more of APLP2, PKP4, HLA-A, CD41, CD97, ITGB5, ITGA2B, Integrin αVβ5, PI4K2A, TMEM120A, PTK7A, Ceruloplasmin, PTPRF, SLC16A1, SLC7A5, ATP1B1, ATP2B4, PIP4K2A, and P142A. In some embodiments, APLP2, PKP4, and HLA-A, and one or more additional proteins mediate binding and/or internalization of the ctDNA, derivative thereof, synthetic delivery system, or polynucleotide.

### Target Cells

Compositions and methods disclosed herein can relate to target cells. For example, an antagonist disclosed herein can be used to reduce uptake of ctDNA by target cells, a biological sample disclosed herein can comprise target cells and can be assayed to determine a level of expression of a receptor associated with uptake of ctDNA, a synthetic delivery system, polynucleotide, or cargo can be delivered to a target cell, or an assay can be performed on target cells to identify a receptor that mediates uptake of ctDNA.

Compositions and methods disclosed herein can comprise blocking binding or uptake of a ctDNA by a target cell. For example, a target cell can comprise a receptor, and the target cell, ctDNA, or receptor can be contacted with an antagonist disclosed herein, thereby reducing binding of the ctDNA to the target cell and/or reducing uptake of the ctDNA by the target cell.

Compositions and methods disclosed herein can comprise delivering a cargo to a target cell. For example, a target cell can comprise a receptor, and uptake of the synthetic delivery system or cargo by the target cell can be induced by contacting the target cell or the receptor on the target cell with a synthetic delivery system and/or polynucleotide disclosed herein. Methods disclosed herein can comprise identifying a receptor that mediates uptake of a cargo by a target cell.

A target cell can be a primary cell. A target cell can be a cancer cell, for example, a hematologic tumor cell or a solid tumor cell. A target cell can be a cell line.

A target cell can be a eukaryotic cell. A target cell can be a mammalian cell. A target cell can be a human cell.

A target cell can be a leukocyte, such as a lymphoid or myeloid cell. A target cell can be a plasma cell. A target cell can be a multiple myeloma cell. A target cell can be a B cell. A target cell can be a T cell. A target cell can be a lymphocyte. A target cell can be a monocyte or a macrophage. A target cell can be a neoplastic myeloid cell. A target cell can be a neoplastic lymphoid cell. A target cell can be a multiple myeloma cell. A target cell can be a leukemia cell. A target cell can be a lymphoma cell.

A target cell can be a pancreatic cell. A target cell can be an exocrine pancreatic cell. A target cell can be an endocrine pancreatic cell. A target cell can be a pancreatic cancer cell. A target cell can be a bile duct cell. A target cell can be a gallbladder cell.

A target cell can be a gastrointestinal cell. A target cell can be, for example, an intestine cell, such as a colon cell. A target cell can be a colorectal cancer cell.

A target cell can be, for example, a lymphoma, leukemia, lung cancer, colon cancer, or brain cancer cell.

In some embodiments, a ctDNA, synthetic delivery system, or polynucleotide disclosed herein or a component thereof (e.g., a recognition sequence) can specifically or preferentially mediate binding, uptake, and/or genomic integration of a target cell. For example, in some embodiments, a synthetic delivery system disclosed herein can bind to, enter, localize to the nucleus of, and/or integrate into the genome of a target cell disclosed herein at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold more than a control cell. The control cell can be, for example, a different cell or tissue type. The control cell can be, for example, a corresponding cell type that is a non-cancer cell, as compared to a target cell that is a cancer cell.

In some embodiments, specificity for a target cell can reduce off-target and unwanted side effects, for example, as compared to conventional cargo delivery system (e.g., nanoparticles or viral vectors).

### Synthetic Delivery System

Compositions and methods provided herein include synthetic delivery systems that utilize polynucleotides to facilitate binding, uptake, nuclear trafficking, and/or genomic integration of a component of the synthetic delivery system, for example, a cargo. The compositions and methods can be used for delivery of a cargo to cells, including specific cell types and cancer cells. Synthetic delivery systems disclosed herein can provide for cell-, tissue-, and/or organ-specific targeting, delivery, binding, uptake, nuclear localization and/or genomic integration, e.g., of a cargo.

A synthetic delivery system disclosed herein can comprise a polynucleotide. A synthetic delivery system can comprise, for example, a polynucleotide and a cargo. A polynucleotide can be or can comprise double stranded DNA (dsDNA). In some embodiments, a polynucleotide comprises single stranded DNA. In some embodiments, a polynucleotide comprises RNA.

A polynucleotide of the disclosure or a part thereof (for example, recognition and/or integration sequence) can be from or derived from a nucleic acid sequence of a biological sample (e.g., blood, plasma, or tissue sample of a subject, such as a human). A polynucleotide of the disclosure or a part thereof (for example, recognition and/or integration sequence) can be from or derived from a cancerous cell or tissue.

A polynucleotide in a synthetic delivery system or a part thereof (for example, recognition and/or integration sequence) can be from or derived from a circulating tumor DNA (ctDNA), for example, from a human. A ctDNA can comprise double stranded DNA (dsDNA). In some embodiments, a ctDNA comprises single stranded DNA. Such a ctDNA or a fragment thereof can comprise a nucleic acid sequence that can have the ability to target a certain cell or cell population, and induce binding, uptake, nuclear localization, and/or genomic integration of a polynucleotide or cargo disclosed herein.

A polynucleotide in a synthetic delivery system that delivers a cargo to a target cell can comprise a nucleic acid sequence that is from or derived from ctDNA. The ctDNA can originate from the same cell type or tissue type as the target cell. A polynucleotide used to deliver a cargo to a target cell can comprise a nucleic acid sequence that is from or derived from ctDNA of substantially the same cell type or substantially the same tissue type as the target cell. A polynucleotide used to deliver a cargo to a target cell can comprise a nucleic acid sequence that is from or derived ctDNA of a substantially similar cell type or substantially similar tissue type as the target cell. For example, a ctDNA from multiple myeloma or a component (e.g., subsequence or recognition sequence) therefrom can be used to generate a synthetic delivery system for delivery, binding, uptake, nuclear localization, and/or genomic integration of a cargo to a multiple myeloma target cell or a related target cell or target cell type. ctDNA from pancreatic cancer or a component (e.g., subsequence or recognition sequence) therefrom can be used to generate a synthetic delivery system for delivery, binding, uptake, nuclear localization, and/or genomic integration of a cargo to a pancreatic cancer target cell or a related target cell or target cell type. ctDNA from colorectal cancer or a component (e.g., subsequence or recognition sequence) therefrom can be used to generate a synthetic delivery system for delivery, binding, uptake, nuclear localization, and/or genomic integration of a cargo to a colorectal cancer target cell or a related target cell or target cell type. ctDNA from lung cancer or a component (e.g., subsequence or recognition sequence) therefrom can be used to generate a synthetic delivery system for delivery, binding, uptake, nuclear localization, and/or genomic integration of a cargo to a lung cancer target cell or a related target cell or target cell type.

A ctDNA, synthetic delivery system, or polynucleotide can comprise a recognition sequence that can provide for cell-specific targeting and/or uptake of the ctDNA, polynucleotide, synthetic delivery system, or cargo. In some embodiments, the recognition sequence binds to a receptor that mediates or is associated with uptake of ctDNA. In some embodiments, the recognition sequence of a ctDNA, synthetic delivery system, or polynucleotide specifically or preferentially binds to a receptor that is associated with uptake of ctDNA. In some embodiments, the recognition sequence results in preferential binding of a ctDNA, synthetic delivery system, polynucleotide, or cargo to a target cell as compared to a control cell. In some embodiments, the recognition sequence results in preferential uptake of a ctDNA, synthetic delivery system, polynucleotide, or cargo by a target cell as compared to a control cell. In some embodiments, the recognition sequence results in preferential binding, uptake, nuclear localization, and/or genomic integration of a ctDNA, synthetic delivery system, polynucleotide, or cargo by a target cell as compared to a control cell. The control cell can be, for example, a cell of a different cell type. In some embodiments, the target cell is a cancer cell and the control cell is a non-cancer cell. In some embodiments, the target cell is a cancer cell of a first cell type and the control cell is a cancer cell of a second cell type. In some embodiments, the target cell is a cancer cell of a first cell type and the control cell is a non-cancer cell of a second cell type.

In some embodiments, the recognition sequence of a ctDNA, synthetic delivery system, or polynucleotide facilitates at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold higher binding to a receptor disclosed herein or to a target cell (e.g., population thereof) disclosed herein as compared to a control ctDNA, synthetic delivery system, or polynucleotide that lacks the recognition sequence.

In some embodiments, the recognition sequence of a ctDNA, synthetic delivery system, or polynucleotide facilitates at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold higher uptake of the ctDNA, synthetic delivery system, polynucleotide, or cargo by target cells as compared to a control ctDNA, synthetic delivery system, or polynucleotide that lacks the recognition sequence.

In some embodiments, the recognition sequence of a ctDNA, synthetic delivery system, or polynucleotide facilitates at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold higher nuclear localization of the ctDNA, synthetic delivery system, polynucleotide, or cargo in target cells as compared to a control ctDNA, synthetic delivery system, or polynucleotide that lacks the recognition sequence.

In some embodiments, the recognition sequence of a ctDNA, synthetic delivery system, or polynucleotide facilitates at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold higher genomic integration of the ctDNA, synthetic delivery system, polynucleotide, or cargo by target cells as compared to a control ctDNA, synthetic delivery system, or polynucleotide that lacks the recognition sequence.

In an example, a multiple myeloma (MM)-derived recognition sequence can target binding and/or uptake of a synthetic delivery system, polynucleotide, and/or cargo by MM cells but not, e.g. pancreatic cells. A pancreatic cancer (PC)-derived recognition sequence can target binding and/or uptake of a synthetic delivery system, polynucleotide, and/or cargo by pancreatic cancer cells, but not, e.g., multiple myeloma cells.

A recognition sequence can be derived from a ctDNA. For example, a recognition sequence can be a fragment of a ctDNA identified by sequencing a ctDNA from a subject, e.g., from plasma of a human subject with a cancer. A recognition can be derived from a ctDNA that originates from a lymphoma, leukemia, lung cancer, colon cancer (e.g., colorectal cancer), brain cancer, multiple myeloma, pancreatic cancer, hematologic cancer, or solid cancer.

A synthetic delivery system or polynucleotide can comprise any suitable number of recognition sequences, for example, one, two, three, four, or five recognition sequences. In some embodiments, a synthetic delivery system or polynucleotide comprises one recognition sequence. In some embodiments, a synthetic delivery system or polynucleotide comprises two recognition sequences, for example, a first recognition sequence at a 5′ region of the polynucleotide and a second recognition sequence at a 3′ region of the polynucleotide, optionally separated by a non-recognition sequence or component, such as a nucleic acid cargo.

In some embodiments, a synthetic delivery system or polynucleotide can comprise at least two, at least three, at least four, at least five, or another suitable number of recognition sequences at either the 3′ or the 5′ end, or both, of a polynucleotide. A composition of the disclosure can comprise a plurality of such polynucleotides, and each polynucleotide can target one or more cell type(s). Accordingly, the methods and compositions of the present disclosure can be used to target one or more different target cells, one or more different target cell population(s), and/or one or more different target tissue(s), e.g., simultaneously.

A polynucleotide or a nucleic acid sequence (e.g., a recognition sequence, integration sequence, first nucleic acid sequence, or second nucleic acid sequence) can comprise a transposable element (TE) or a derivative thereof. A transposable element can comprise a transposon or a derivative thereof. A transposable element can comprise a retrotransposon or a derivative thereof. A transposable element or transposon can be any suitable class, subclass, family, subfamily, type, or the like, including those disclosed herein. A transposable element or transposon can be a variant or derivative of a transposable element of a given class, subclass, family, subfamily, or type, including those disclosed herein.

A transposable element or functional fragment thereof can be from or derived from, for example, a class I or class II transposon. In some embodiments, a transposable element comprises or is derived from a short interspersed nuclear elements (SINE), long interspersed nuclear elements (LINE), ERVL, or ERVK transposable element.

In some embodiments, a ctDNA, polynucleotide, or a nucleic acid sequence (e.g., a recognition sequence, integration sequence, first nucleic acid sequence, or second nucleic acid sequence) comprises a transposable element that is an AluSp, MER11C, AluY, 2L2a, ALluY, ALR/Alpha, ALU, AluJb, AluS1, AluSc8, AluSg, ALUSg2, AluSq, AluSq2, ALUSx, AluSx, AluSxl, AluSz, AluSz6, AluYc3, ASLUSq2, ERVK/LTR, ERVL, ERVL-MaLR, FLAM_C and AluY, HERV17-int, HERV9N-int, L1M1, L1MB3, L1ME4b, L1MEg, L1P1, L1P3, L1PA10, L1PA15, L1PA7, L1PB4, L2a, L2a/LTR40b/MLT1J2, LINE/L1, LINE/L2, LTR, LTR/ERV1, LTR/ERVL, LTR/ERVL-MaLR, LTR/Gypsy, LTR41C, LTR81B, Mam_R4, Mamr4, Many, MER11B, MER41E, MIR, MIRB, MIRc, MIRc-part L2, MLT1J2, MLT2B1, MTL1J2, MTL2B4, parAluSp-FULLMTL1J2, REP522, Satellite/centr, SINE/Alu, SINE/MIR, THE1A, THE1B, THE1C, or Tigger3a transposable element, or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is an AluSp transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is a MER11C transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is a AluSx transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is a MTL1J transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is a AluSg2 transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is a THE1A transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is an AluJb transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is a MTL2B4 transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is a L2a transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is a MTL1J2 transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is an AluSq transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is a L1MB3 transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is a THE1C transposable element or a derivative thereof.

In some embodiments, a ctDNA, polynucleotide, or a nucleic acid sequence (e.g., a recognition sequence, integration sequence, first nucleic acid sequence, or second nucleic acid sequence) comprises a transposable element that is an AluY transposable element or a derivative thereof.

In some embodiments, upon contacting, arrival, or internalization at the host cell, a transposable element disclosed herein co-opts the cellular retrotransposition machinery to integrate into the target (e.g., cancer) cell genome. In some embodiments, tissue-specific delivery, uptake, and/or integration can be determined by the retrotransposon's sequence and the host's expression of the retrotransposition machinery.

A ctDNA or polynucleotide can comprise an integration sequence that facilitates integration of the ctDNA, polynucleotide, synthetic delivery system, or cargo, into the genome of a target cell. The integration sequence can comprise, for example, a transposon integration signal. In some embodiments, a polynucleotide lacks an integration sequence.

A polynucleotide can comprise a transposon sequence or a functional fragment thereof. A polynucleotide can comprise two or more transposon sequences or functional fragments thereof. A transposon sequence or functional fragment thereof can be from or derived from, for example, a class II transposon.

A polynucleotide can comprise a first nucleic acid sequence that is from or derived from ctDNA. The ctDNA can originate from a cancer cell of the same, substantially the same, or a substantially similar cell type or tissue type as a target cell.

The first nucleic acid sequence can be in a 3′ region of the polynucleotide. The polynucleotide can further comprise a second nucleic acid sequence, for example, that was identified in, is from, or is derived from ctDNA. The ctDNA can be from or derived from the same, substantially the same, or a substantially similar cell type or tissue type as the target cell. The ctDNA can be the same ctDNA as that from which the first nucleic acid sequence is from or derived from. In some embodiments, the ctDNA is different than the ctDNA from which the first nucleic acid sequence is from or derived from. The second nucleic acid can be in a 5′ region of the polynucleotide. The first nucleic acid sequence can be or can comprise a nucleic acid sequence with at least 70%, at least 80%, at least 90%, at least 95%, or at least 99% sequence identity to any one or more of SEQ ID NOs: 1-277 and SEQ ID NOs: 282-349, or a fragment thereof. The second nucleic acid sequence can be or can comprise a nucleic acid sequence with at least 70%, at least 80%, at least 90%, at least 95%, or at least 99% sequence identity to any one or more of SEQ ID NOs: 1-277 and SEQ ID NOs: 282-349, or a fragment thereof. The first and/or the second nucleic acid sequence can comprise a functional fragment (for example, a recognition sequence or integration sequence) that is from or derived from any one or more of SEQ ID NOs: 1-277 and SEQ ID NOs: 282-349.

The first nucleic acid sequence can be in a 5′ region of the polynucleotide. The polynucleotide can further comprise a second nucleic acid sequence, for example, that was identified in, is from, or is derived from ctDNA. The ctDNA can be from or derived from the same, substantially the same, or a substantially similar cell type or tissue type as the target cell. The ctDNA can be the same ctDNA as that from which the first nucleic acid sequence is from or derived from. In some embodiments, the ctDNA is different than the ctDNA from which the first nucleic acid sequence is from or derived from. The second nucleic acid can be in a 3′ region of the polynucleotide. The first and/or the second nucleic acid sequence can comprise a functional fragment (for example, a recognition sequence or integration sequence) that is from or derived from any suitable sequence disclosed herein. The first nucleic acid sequence can be or can comprise a nucleic acid sequence with at least 70%, at least 80%, at least 90%, at least 95%, or at least 99% sequence identity to any one or more of SEQ ID NOs: 1-277 and SEQ ID NOs: 282-349, or a fragment thereof. The second nucleic acid sequence can be or can comprise a nucleic acid sequence with at least 70%, at least 80%, at least 90%, at least 95%, or at least 99% sequence identity to any one or more of SEQ ID NOs: 1-277 and SEQ ID NOs: 282-349, or a fragment thereof. The first and/or the second nucleic acid sequence can comprise a functional fragment (for example, a recognition sequence or integration sequence) that is from or derived from any one or more of SEQ ID NOs: 1-277 and SEQ ID NOs: 282-349.

In some embodiments, one portion (e.g., about half) of a nucleic acid sequence can be linked to the 5′ end of a nucleic acid cargo, gene, or sequence of interest, and the other portion (e.g., about half) of the sequence can be linked 3′ end of the nucleic acid cargo, gene, or sequence of interest. In some embodiments, a nucleic acid sequence can be linked directly or indirectly to a nucleic acid cargo, gene, or sequence of interest. For example, a first sequence can be linked directly to the 5′ end of a nucleic acid sequence comprising the nucleic acid cargo, gene, or sequence of interest, and a second sequence can be linked indirectly to the 3′ end of the nucleic acid sequence comprising the nucleic acid cargo, gene, or sequence of interest. In some embodiments, a composition of the present disclosure can comprise a guide sequence that is directly linked to a nucleic acid cargo, gene, or sequence of interest at one terminus (e.g., 3′ end) and directly linked to a recognition sequence or integration sequence at the other terminus (e.g., 5′ end), thereby indirectly connecting the integration or insertion sequence and the nucleic acid cargo, gene, or sequence of interest.

In some embodiments, a ctDNA, synthetic delivery system, polynucleotide, or nucleic acid sequence comprises a disclosed minimal degree of sequence identity to a sequence disclosed herein, or a fragment thereof. The fragment thereof can be a functional fragment, for example, that act as a recognition sequence or integration sequence as disclosed herein.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 10 consecutive nucleotides of any one of SEQ ID NOs: 1-277 and 282-349.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 15 consecutive nucleotides of any one of SEQ ID NOs: 1-277 and 282-349.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 20 consecutive nucleotides of any one of SEQ ID NOs: 1-277 and 282-349.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 30 consecutive nucleotides of any one of SEQ ID NOs: 1-277 and 282-349.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 50 consecutive nucleotides of any one of SEQ ID NOs: 1-277 and 282-349.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 75 consecutive nucleotides of any one of SEQ ID NOs: 1-277 and 282-349.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 100 consecutive nucleotides of any one of SEQ ID NOs: 1-277 and 282-349.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 200 consecutive nucleotides of any one of SEQ ID NOs: 1-277 and 282-349.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 300 consecutive nucleotides of any one of SEQ ID NOs: 1-277 and 282-349.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to any one of SEQ ID NOs: 1-277 and 282-349.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises, consists essentially of, or consists of the nucleotide sequence of any one of SEQ ID NOs: 1-277 and 282-349.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises, consists essentially of, or consists of a nucleic acid sequence with at most about 70%, at most about 75%, at most about 80%, at most about 85%, at most about 90%, at most about 95%, at most about 97.5. or at most about 99% sequence identity to any one of SEQ ID NOs: 1-277 and 282-349.

In some embodiments, a polynucleotide or a nucleic acid sequence (for example, a ctDNA or a synthetic delivery system or a part thereof, such as a recognition sequence, integration sequence, first nucleic acid sequence, or second nucleic acid sequence) comprises a nucleic acid sequence with one or more nucleotide insertions, deletions, or substitutions compared to any one of SEQ ID NOs: 1-277 and 282-349.

For example, the polynucleotide or a nucleic acid sequence can comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 nucleotide insertions relative to any one of SEQ ID NOs: 1-277 and 282-349.

In some embodiments, the polynucleotide or a nucleic acid sequence comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions relative to any one of SEQ ID NOs: 1-277 and 282-349.

In some embodiments, the polynucleotide or a nucleic acid sequence comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions relative to any one of SEQ ID NOs: 1-277 and 282-349.

The one or more insertions can be at the 5′ end, 3′ end, within the sequence, or a combination thereof. The one or more insertions can be contiguous, non-contiguous, or a combination thereof.

In some embodiments, the polynucleotide or a nucleic acid sequence comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 75, at least 100, at least 125, at least 150, at least 175, at least 200, at least 250, at least 300, at least 400, at least 500, at least 600, or at least 700 nucleotide deletions relative to any one of SEQ ID NOs: 1-277 and 282-349.

In some embodiments, the polynucleotide or a nucleic acid sequence comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or most 50, at most 75, at most 100, at most 125, at most 150, at most 175, at most 200, at most 250, at most 300, at most 400, at most 500, at most 600, or at most 700 nucleotide deletions relative to any one of SEQ ID NOs: 1-277 and 282-349.

In some embodiments, the polynucleotide or a nucleic acid sequence comprises 1, 2, 3, 4, 5, 6, 7, 8, 9, 10, 11, 12, 13, 14, 15, 16, 17, 18, 19, 20, 25, 30, 35, 40, 45, or50 nucleotide deletions relative to any one of SEQ ID NOs: 1-277 and 282-349.

The one or more deletions can be at the 5′ end, 3′ end, within the sequence, or a combination thereof. The one or more deletions can be contiguous, non-contiguous, or a combination thereof.

In some embodiments, the polynucleotide or a nucleic acid sequence comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 nucleotide substitutions relative to any one of SEQ ID NOs: 1-277 and 282-349.

In some embodiments, the polynucleotide or a nucleic acid sequence comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide substitutions relative to any one of SEQ ID NOs: 1-277 and 282-349.

In some embodiments, the polynucleotide or a nucleic acid sequence comprises 1, 2, 3, 4, 5, 6, 7, 8, 9, 10, 11, 12, 13, 14, 15, 16, 17, 18, 19, 20, 25, 30, 35, 40, 45, or50 nucleotide substitutions relative to any one of SEQ ID NOs: 1-277 and 282-349.

The one or more substitutions can be at the 5′ end, 3′ end, within the sequence, or a combination thereof. The one or more substitutions can be contiguous, non-contiguous, or a combination thereof.

A polynucleotide or a nucleic acid sequence disclosed herein can be from about 50 base pairs (bp) to about 1000 bp in length. A polynucleotide or a nucleic acid sequence disclosed herein can be from about 100 base pairs (bp) to about 900 bp in length. A polynucleotide or a nucleic acid sequence disclosed herein can be from about 200 bp to about 800 bp in length. A polynucleotide or a nucleic acid sequence disclosed herein can be from about 300 bp to about 700 bp in length. A polynucleotide or a nucleic acid sequence disclosed herein can be from about 400 bp to about 600 bp in length. A polynucleotide or a nucleic acid sequence disclosed herein can be at least about 50 bp in length. A polynucleotide or a nucleic acid sequence disclosed herein can be at least about 100 bp in length. A polynucleotide or a nucleic acid sequence disclosed herein can be at least about 200 bp in length. A polynucleotide or a nucleic acid sequence disclosed herein can be at least about 300 bp in length. A polynucleotide or a nucleic acid sequence disclosed herein can be at least about 400 bp in length. A polynucleotide or a nucleic acid sequence disclosed herein can be at least about 500 bp in length. A polynucleotide or a nucleic acid sequence disclosed herein can be at least about 1000 bp in length.

A polynucleotide or a nucleic acid sequence disclosed herein can be at most about 50 bp in length. A polynucleotide or a nucleic acid sequence disclosed herein can be at most about 100 bp in length. A polynucleotide or a nucleic acid sequence disclosed herein can be at most about 200 bp in length. A polynucleotide or a nucleic acid sequence disclosed herein can be at most about 300 bp in length. A polynucleotide or a nucleic acid sequence disclosed herein can be at most about 400 bp in length. A polynucleotide or a nucleic acid sequence disclosed herein can be at most about 500 bp in length. A polynucleotide or a nucleic acid sequence disclosed herein can be at most about 1000 bp in length. A polynucleotide or a nucleic acid sequence disclosed herein can be at most about 5000 bp in length. A polynucleotide or a nucleic acid sequence disclosed herein can be at most about 10000 bp in length.

In some embodiments, the polynucleotide or a nucleic acid sequence disclosed herein (e.g., recognition sequence, integration sequence, first nucleic acid sequence, or second nucleic acid sequence) is at least 10 base pairs in length. In some embodiments, the polynucleotide or a nucleic acid sequence disclosed herein is at least 20 base pairs in length. In some embodiments, the polynucleotide or a nucleic acid sequence disclosed herein is at least 50 base pairs in length. In some embodiments, the polynucleotide or a nucleic acid sequence disclosed herein is at least 100 base pairs in length. In some embodiments, the polynucleotide or a nucleic acid sequence disclosed herein is at least 200 base pairs in length. In some cases, the polynucleotide or a nucleic acid sequence disclosed herein can be at least 400 base pairs in length.

In some embodiments, the polynucleotide or a nucleic acid sequence disclosed herein (e.g., recognition sequence, integration sequence, first nucleic acid sequence, or second nucleic acid sequence) is from about 10 base pairs to about 100 base pairs in length. In some embodiments, the polynucleotide or a nucleic acid sequence disclosed herein can be from about 10 base pairs to about 200 base pairs in length. In some embodiments, the polynucleotide or a nucleic acid sequence disclosed herein can be from about 20 base pairs to about 100 base pairs in length. In some embodiments, the polynucleotide or a nucleic acid sequence disclosed herein can be from about 100 base pairs to about 200 base pairs in length. In some embodiments, the polynucleotide or a nucleic acid sequence disclosed herein can be from about 100 base pairs to about 500 base pairs in length. In some embodiments, the polynucleotide or a nucleic acid sequence disclosed herein can be from about 200 base pairs to about 800 base pairs in length. In some embodiments, the polynucleotide or a nucleic acid sequence disclosed herein can be from about 400 base pairs to about 2,000 base pairs in length. In some embodiments, the polynucleotide or a nucleic acid sequence disclosed herein can be from about 400 base pairs to about 5,000 base pairs in length. In some embodiments, the polynucleotide or a nucleic acid sequence disclosed herein can be from about 1,500 base pairs to about 7,200 base pairs in length. In some embodiments, the polynucleotide or a nucleic acid sequence disclosed herein can be from about 1,900 base pairs to about 5,800 base pairs in length. In some embodiments, the polynucleotide or a nucleic acid sequence disclosed herein can be from about 2,000 base pairs to about 10,000 base pairs in length. In some embodiments, the polynucleotide or a nucleic acid sequence disclosed herein can be from about 5,000 base pairs to about 15,000 base pairs in length. In some embodiments, the polynucleotide or a nucleic acid sequence disclosed herein can be from about 600 base pairs to about 16,900 base pairs in length. In some embodiments, the polynucleotide or a nucleic acid sequence disclosed herein can be from about 8,000 base pairs to about 18,000 base pairs in length. In some embodiments, the polynucleotide or a nucleic acid sequence disclosed herein can be from about 10,000 base pairs to about 20,000 base pairs in length. In some embodiments, the polynucleotide or a nucleic acid sequence disclosed herein can be from about 400 base pairs to about 20,000 base pairs in length.

In some embodiments, a ctDNA, polynucleotide, or synthetic delivery system comprises a nucleic acid sequence has at least 70%, at least 80%, at least 90%, at least 95%, or at least 99% sequence identity to any one or more of SEQ ID NOs: 1-277 or SEQ ID NOs: 282-349, or at least 70%, at least 80%, at least 90%, at least 95%, or at least 99% sequence identity to a fragment thereof.

A polynucleotide in a synthetic delivery system or a part thereof (for example, recognition and/or integration sequence) can comprise a nucleic acid sequence with at least 70%, at least 80%, at least 90%, at least 95%, or at least 99% sequence identity to any one or more of SEQ ID NOs: 1-277 and SEQ ID NOs: 282-349, or a fragment thereof. A recognition sequence can comprise a nucleic acid sequence with at least 70%, at least 80%, at least 90%, at least 95%, or at least 99% sequence identity to any one or more of SEQ ID NOs: 1-277 and SEQ ID NOs: 282-349, or a fragment thereof (for example, a functional fragment that an act as a recognition sequence as disclosed herein). An integration sequence can comprise a nucleic acid sequence with at least 70%, at least 80%, at least 90%, at least 95%, or at least 99% sequence identity to any one or more of SEQ ID NOs: 1-277 and SEQ ID NOs: 282-349, or a fragment thereof (for example, a functional fragment that an act as an integration sequence as disclosed herein).

In some embodiments, the polynucleotide comprises at least one substitution relative to any one of SEQ ID NOs: 1-277 and SEQ ID NOs: 282-349. In some embodiments, the polynucleotide comprises at least 10 substitutions relative to any one of SEQ ID NOs: 1-277 and SEQ ID NOs: 282-349. In some embodiments, the polynucleotide comprises at least 50 substitutions relative to any one of SEQ ID NOs: 1-277 and SEQ ID NOs: 282-349. In some embodiments, the polynucleotide comprises at least 100 substitutions relative to any one of SEQ ID NOs: 1-277 and SEQ ID NOs: 282-349. In some embodiments, the polynucleotide comprises at least 250 substitutions relative to any one of SEQ ID NOs: 1-277 and SEQ ID NOs: 282-349. In some embodiments, the polynucleotide comprises from about 1 to about 20 substitutions relative to any one of SEQ ID NOs: 1-277 and SEQ ID NOs: 282-349. In some embodiments, the polynucleotide comprises from about 20 to about 100 substitutions relative to any one of SEQ ID NOs: 1-277 and SEQ ID NOs: 282-349. In some embodiments, the polynucleotide comprises from about 50 to about 250 substitutions relative to any one of SEQ ID NOs: 1-277 and SEQ ID NOs: 282-349. In some embodiments, the polynucleotide comprises from about 150 to about 500 substitutions relative to any one of SEQ ID NOs: 1-277 and SEQ ID NOs: 282-349. In some embodiments, the polynucleotide comprises from about 250 to about 700 substitutions relative to any one of SEQ ID NOs: 1-277 and SEQ ID NOs: 282-349. In some embodiments, the polynucleotide comprises from about 500 to about 750 substitutions relative to any one of SEQ ID NOs: 1-277 and SEQ ID NOs: 282-349. In some embodiments, the polynucleotide comprises from about 500 to about 1,000 substitutions relative to any one of SEQ ID NOs: 1-277 and SEQ ID NOs: 282-349.

In some embodiments, a synthetic delivery system of the disclosure can be functional without requiring a commonly used delivery vector or delivery system, e.g., as “naked” DNA. For example, in some embodiments a synthetic delivery system does not require or utilize a viral vector, a nanoparticle, a lipid nanoparticle, a liposome, an exosome, a dendrimer, a gene gun, or electroporation.

In some embodiments, targeted cargo delivery via synthetic delivery systems disclosed herein can significantly reduce off-target and unwanted adverse effects associated with alternate delivery systems, for example, non-targeted delivery systems.

In some embodiments, a synthetic delivery system of the disclosure can be combined with or utilize a vector, such as a viral vector, a nanoparticle, a lipid nanoparticle, a liposome, an exosome, or a dendrimer.

In some embodiments, a candidate polynucleotide for use in a synthetic delivery system comprises a full length transposable element (e.g., retrotransposon). In some embodiments, candidate polynucleotide for use in a synthetic delivery system comprises a high rate of identified mutations, or more than one identified mutation (e.g., SNV), for example, favoring higher numbers of mutations relative to a reference sequence. The mutation can be shared between ctDNA-target cell coculture conditions and ctDNA alone, and not present or substantially not present in the genome of the untreated control cells. In some embodiments, a candidate polynucleotide for use in a synthetic delivery system is not located in an intron of a gene, or if present in an intron, is not close to an exon (e.g., at least 3 kb, 5 kb, or 10 kb from an exon).

In some embodiments, a synthetic delivery system disclosed herein does not elicit, substantially does not elicit, or elicits a reduced immunogenic response compared to an alternate delivery system, such as a viral vector.

In some embodiments, a synthetic delivery system disclosed herein comprises a conjugation moiety. The conjugation moiety can be attached at the 5′ end, the 3′ end, or at an internal site along the length of a polynucleotide. The conjugation moiety can be a nucleotide analogue (such as bromodeoxyuridine). The conjugation moiety can be a conjugating functional group. The conjugating functional group can be an azido group or an alkyne group. A conjugation moiety can further be derivatized through a chemical reaction such as click chemistry or any other bioconjugation reaction. The click chemistry can comprise a copper(I)-catalyzed [3+2]-Huisgen 1,3-dipolar cyclo-addition of alkynes and azides leading to 1,2,3-triazoles. The click chemistry can be a copper free variant of a reaction. The click chemistry can be an inverse electron-demand Diels-Alder reaction between a trans-cyclooctadiene and a tetrazine.

In some embodiments, the conjugation moiety as used herein can comprise a hapten group. A hapten group can include digoxigenin, 2,4-dinitrophenyl, biotin, avidin, or be selected from azoles, nitroaryl compounds, benzofurazans, triterpenes, ureas, thioureas, rotenones, oxazoles, thiazoles, coumarins, cyclolignans, heterobiaryl compounds, azoaryl compounds or benzodiazepines. A hapten group can include biotin. A nucleic acid comprising the conjugating moiety can further be linked to a second nucleic acid, a fluorescent moiety (such as a dye such as a quantum dot), or a conjugating partner such as a polymer (such as PEG), a macromolecule (such as a carbohydrate, a lipid, a polypeptide), for example.

TABLE 1 provides sequences of illustrative polynucleotides that can be used in a synthetic delivery system disclosed herein, for example, for delivery to cancer cells, e.g., hematologic cancer cells, such as multiple myeloma cells, B cells, and/or plasma cells.

TABLE 1

| SEQ ID NO | Description | Sequence |
|---|---|---|
| 283 | ALUSx | TTTTTCTTCTCTCTTTTTTTTTTATTTTTTGAGATGGAGTT |
| | | TCACTCTGTTGCCCAGGCTGGAGTGCAATGGTGCAGTCT |
| | | CAGCTCACTGCAACCTCCACCTCCCGGGTTTGAGCAGTG |
| | (ASLUSq2) | ATTCTGCCTCAGCCTCCCAAGTAGCTGGGATTACAGGCA |
| | | CCCACCACCATGCCTGGCTAATTTTTGTATTTTTAGTAGA |
| | | GATGGGGTTTCACCATGTTGGCCAGGCTGGTCTCGAACT |
| | | CCTGACCTCAGGTGATCCACCTGCCTCGGCCTCCCAAAG |
| | | TGCTGAAATTACAGGCATGAGCCACCGCACCTGGCC |
| 284 | L1MB3 | TTTTTGTATTTTTAGTAGAGATGGGGTTTCACCATGTTGG |
| | | CCAGGCTGGTCGAACTCCTGACCTCAGGTGATCCACCtG |
| | | CCTCGGCCTCCCAAAGTGCTGAaATTACAGGcaTGAGCCA |
| | | CCCCCAGAACTCTTCTCTTTCCCAAAACTAAAATTCTGC |
| | | ACCCATTGAACAGTAACTCCCCATTTCTCCTTTCCCCTAA |
| | | CTCCTGGCATCCACCATTCTGTTTTCTGTCTTTACGAATT |
| | | TTACTACCCTTGGTACTTCCTTTAAGTGGAATCATACAAT |
| | | ATTTTTCTTTTTCTGACTGGCTTATTTCACTAAGCATAAT |
| | | ATTTTTAAGGTTCAAGCATTTTGTAGCATATGTCAGAATT |
| | | TCCTTTCTTTTTAAAGCTCAATAATATTCCATTGTATGtAT |
| | | ATACCACATTTTACTTAACCATTCATCCgTCAGTGGACAc |
| | | TTGGGTTACTTCCAAcTTTTAGCTATTGTGAATAATGCTG |
| | | CTATGAACATGAATGTACAAATATCTnTTCGAGnCCCTGC |
| | | TTTCAATTCTTTTGGGTATATACCCAGAAGTGGAATTGCT |
| | | GGATCATATGGTAATTCTATTTTTAATTTTTTGAGGAACC |
| | | GCCATACTGTTTTCCACAGCGGCTGCACCATTTTACATTC |
| | | CCACCAGCAGTGCACAAGGGTTCCAATTTCTCCACATCC |
| | | TCGCCAACACTTGTTATTTTCTGTTTTTTTGATAGTAGCC |
| | | ATCCTAATGGGTGTGAGGTGGTATCTCATTGTGGTTTTG |
| | | ATTTGCATTTCCCTAATGATTAGTGATGTTGAGCATCTTT |
| | | TCATGTGCTTATTGGCCATTTGTATATCTTCTTTGGAGAA |
| | | ATGTCTATTCAAGTCCTTTGCCCATTTTTnAATCGGGTTG |
| | | TTTGTTTTTTTGTTGTTGAGTTGTAGGAGTTCTTTATATAT |
| | | TCTGGATATTAA |
| 285 | L2a | TCTCACCACCTCTACTACTGCCTATCTGCATCTGGGTGCA |
| | (2L2a) | CGTCCTCTGCTATTCCTTCTGTTTCTACAGAAGCTGTCCA |
| | | TATTTCTAAGGCTAATCCCTTGCATTTTGGTACCAGACTT |
| | | CCCTGACCTACACAAAGATGATCACCCCCCAAAATTGCC |
| | | TTTCTTTCCTGCATCAAGTTTTTTCTTCTCTACTGTATTAT |
| | | TCCTATTGCACATTAGCCCAAAAAATGCCGTTGTTTTTCC |
| | | ATCGGTAAAAAAAAAAAATAGAGAGAAAAAGAAAAAT |
| | | AATCCATTGCCGCCAATCTATCTTCTAGTCATTTTTCCAT |
| | | TTCTCTGTTCTCCATTCTGCATAAACCATTTTTACTTGAA |
| | | TTGTTTATACTTGCTGTTTACAACAAACCTCCTCTTTCTC |
| | | TCCACCCCAATCAGATTTTGGTCCACATTCCTCATGCAGT |
| | | GGTCAGTCCTTGGTCCTCAACCTAGTCTAGCCTGCTTGCT |
| | | GGTGGGTGTGCTCTCGGGGCATTTGCTTGTGTTTGCACG |
| | | ACAGCTCAGTCCCTCACTTCTCTCAAGTCTTTGCTAATAA |
| | | CACTCTTGCATCCAAGTCTTCCCTGACCTGCTACTTAAAA |
| | | ACAACTCACCCCCACCCCTAGAAATCCCTATCCTCTTCC |
| | | CTACGCTTTATTTTTCTCCATAATACTTATCACCATGTAG |
| | | CTGAACATGTATTTTACTCACTTTTTTTTATATTGTCCAT |
| | | CTTCCCTCAGTGAATTGAAAGCTCCATGAGGGCAGGAGG |
| | | TTTTGTTTATTGTGTTTACTGCTTTATCCCTAGCACCCAG |
| | | CACAGCATATGTGATATAGTAGACACTCAATACAAATCT |
| | | GATGTGTGTGTGTCT |
| 286 | THE1C | TGATATGGTTTGGCTGTGTCCCCACCTAAATCTCATCTTG |
| | (ERVL-MaLR) | AATTGTAGTTCCCATAATCCCCATGTGTCAtGGGAGGGA |
| | | CCCAGTGGGAGGTAATTTAATCCTGGGGGCAGTTACCCT |
| | | CCTGCTATGTGTGTGTGTGTGTGTGTGTTCTCACGAGATC |
| | | TGATGGTTTTATAAGGGGCTTTTCCCCCTTCGCTCGGCAC |
| | | TTCTCCTTCCTGCCGCCATGTGAAGAAGGACGTGTTTGC |
| | | TTCCCCTTCCGCCATGATTGTAAGTTTCCTGAGGCCTCCC |
| | | CAGCCATGCnGAACTGTGAGTCAATTAAACCTCTTTCCTT |
| | | TATAAATTACCCAGTCTCGGGTGTGTCTTTATTAGCAGC |
| | | GTGAGAACGGACTAATACA |
| 287 | ALUY (ALU) | TTTTGCTCTTCTTTTTTTTTTTTTTTTTGAGAGACGGAGTC |
| | | TCGCTCTGTCTCCCAGGCTGGAGTGCAGTGGCGCGATCT |
| | | CGGCTCACTGCAAGCTCCGCCTCCCGGGTTCACGCCATT |
| | | CTCCTGCCTCAGCCTCCCGAGTAGCTGGGACTACAGGCG |
| | | CCCGCCACCACGCCCGGCTAATTTTTTGTATTTTTAGTAG |
| | | AGACGGGGTTTCACCGTGTTAGCCAGGATGGTCTCGATC |
| | | TCCTGACCTCGTGATCCGCCCGCCTCGGCCTCCCAAAGT |
| | | GCTGGGATTACAGGCGTGAGCCACCGCGCCCGGCC |
| 288 | LIME4b | TATTTACATTATTTTAATAGGTAATGCAATTACATCTTCT |
| | | AAAATTCAGAAAGCATAAAAGTACATAAGAGAAAAACT |
| | | CATCATCCAGCCCTTGCCCTTGAGGACGCTAGTTTCTAC |
| | | CCACACAGCTAAACCTTGTTACAAATTACTTGGGTGTAT |
| | | TACCAGTGGCATTGCCAGGTTTAACAAATAAAAACATAG |
| | | GATTCCTGATTAAGTTTAATTTTCAGATAAATAATTACAT |
| | | GCTTTTTAAGTAGAAGTATTTCCCAAATATTACAAGAGA |
| | | CAATATATTTAAAAAATTGTTTGATATCCAAATTTAACT |
| | | ATGCGTCCTATATTTTGTCTGGCAACCCTAATCCTTCATT |
| | | GTATGGATGTACCCCAGGTTCTTATGCAAATAGAGAAAA |
| | | TATGCACTCTTTTATTATCCTCCGTCCCTTTCTTTTACAG |
| | | ACAATGATAGAATGCTTTATTTAGTACATACGTCATTCT |
| | | GCACCTGTGCGAGTATTTTAGTTAACCACACTTTTGGGG |
| | | ATTGTTCTGCTATCATAAAAGATATTTCTCTTTTTTACCA |
| | | TAATGTATTTCATTAAACCCTCTTTCACAGGTTAGGTCCC |
| | | ATATTTTTCTTTTTTTTTTTTTTTTTGAGAGACGGAGTCTC |
| | | GCTCTGTCTCCCAGGCTGGAGTGCAGTGGCGCGATCTCG |
| | | GCTCACTGCAAGCCTGATAGGTGAAAAATGGTATCTCAG |
| | | TGTAGTTTTAATTTGCATTTCTCTTATTATGAGTGAGGTT |
| | | GAGCATCTTTTCATATGTTTAAGAGCCATTTGTATTTCCT |
| | | TTTCTGTGAACTGTCTGTCCGCCTCCCGGGTTCACGCCAT |
| | | TCTTGGGTTGTTGGTCTTTTTCTTATTGACCTGCCTCAGC |
| | | CTCCCGAGTAGCTGGGAAATCTA |
| 289 | ALUSx | GGTCAGTCGCGGTGGCTCATACTTGTAATCTCAGCACTT |
| | (ALUSg2) | TGGGAGGCCAAGGCGGGTGGATCACCTCAGGTCAGGAG |
| | | TTCAAGACCAGCCTGACCAATGTGGTAAAACCCTGTCTC |
| | | TACTAAAAATACAAAAATTAGCTGGGCGTGGTGGCACA |
| | | TGCCTGTAATCCCAGCTACTCAGGAGGCTGAGGCAGGA |
| | | GAATCGCTTGAACCCGGGAGGCAGAGGTTGCAGTGAGC |
| | | CAAGATAACACCACTGCACTCCAGCCTGGGTGACAGAG |
| | | CAAGACTCCGTCTCAAAAAAAAAAAAAAAAGATCCAGT |
| | | GATAGG |
| 290 | MIR | TATCTATTCACATTTATATGCTCTTAGTCAGAAAATAAA |
| | | ATACCTCTCTTGAAATGGGCCTGACTCCGGAGAGGGAGA |
| | | CCAGATTCACTGCAAGCTCCGCCTCCCGGGTTCCCGCCA |
| | | TTCTCCTGCCTCAGCCTCCCCAGTAGCTCTGACTACAGG |
| | | CGCCCGCCACCACGCCCGGCTAATTTTTTTATTTTTTAGT |
| | | AGAGACGGGGTTTCACCGTGTTAGCCAGGATGGTCTCGA |
| | | TCTCCCAACCTCGTGATCCGCCCGCCTCGGCCTCCCAAA |
| | | GTGCTGGGATTACAGGCATGAGCCACCGCGCCCGGCC |
| 291 | AluY | TTTTTTTTTCTTTTTTTTTTTTTTTCTTTTGAGACGGAGTCT |
| | (ALU) | TGCTCTGTCGCCCAGGCTGGAGTGCAGTGGCGCGATCTC |
| | | GGCTCACTGCAAGCTCCGCCTCCCAGGTTCATGCAATTC |
| | | TCCTGCCTCAGCCTCCCGAGTAGCTGGGACTACAGGCGC |
| | | CCGCCACCACGCCCGGCTAATTTTTTGTATTTTTAGTAGA |
| | | GACGGGGTTTCACCGTGTTAGCCAGGATGGTCTCGATCT |
| | | CCTGACCTCGTGATCCGCCCGCCTCGGCCTCCCAAAGTG |
| | | CTGGGATTACAGGCGTGAGCCACCGCGCCCGGCC |
| 292 | ALluY | TGCTGGGCATGGTGGCTCACGCCTGTAATCCCAGCACTT |
| | | TGGGAGGCCAAGGCGGGCAGATCACGAGGTCAGGAGTT |
| | | CGAGACCATCCTGGCTAACACAGTGAAACCCCGTCTCTA |
| | | CTAAAAATACAAAAAATTAGCCGGGCGTGGTGGCGGGT |
| | | GCCTGTAGTCCCAGCTACTCGGGAGGCTGAGGCAGGAG |
| | | AATGGCGTGAACCCAGGAGGCGGAGCTTGCAGTGAGCT |
| | | GAGATTGCGCCACTGCACTCCAGCCTGGGTGACAGAGC |
| | | GAGACTCCTCTTCAAAAAAAAAAAAAAAAAAAAAAAGA |
| | | CTCAT |
| 293 | AluJb | GGCCGGGCGCGGTGGCTCACGCCTGTAATCCCAGCACTT |
| | (ALU) | TGGGAGGCCGAGGCGGGnGGATCGCTTGAGCCCAGGAG |
| | | TTCGAGACCAGCCTGGGCAACATGGCGAAACCCCGTCTC |
| | | TACAAAAAATACAAAAATTAGCCGGGCGTGGTGGCGCG |
| | | CGCCTGTAGTCCCAGCTACTCGGGAGGCTGAGGCGGGA |
| | | GGATCGCTTGAGCCCGGGAGGTCGAGGCTTCAGTGAGCT |
| | | GAGATCAcgCCACTGCACTCCAGCCTGGGTGACAGAGcgA |
| | | GACTCtGTCTCAAAAAAAATAAATAAAAAATAAAAATAA |
| | | AA |
| 294 | AluSx | GGCCGGGCGCGGTGGCTCACGCCTGTAATCCCAGCACTT |
| | (AluSg) (ALU) | TGGGAGGCCGAGGCGGGCGGATCACCTGAGGTCAGGAG |
| | | TTCGAGACCAGCCTGGCCAACATGGTGAAACCCCGTCTC |
| | | TACTAAAAATACAAAAATTAGCCGGGCGTGGTGGCGCG |
| | | CGCCTGTAATCCCAGCTACTCGGGAGGCTGAGGCAGGA |
| | | GAATCGCTTGAACCCGGGAGGCGGAGGTTTCAGTGAGC |
| | | TGAGATCACGCCACTGCACTCCAGCCTGGGTGACAGAGC |
| | | GAGACTCTGTCTCAAAAATAAATAAATAAATAAAAAAT |
| | | AAAAA |
| 295 | MER11C | TATCGAAAATTCACCCCCGATATTCACGTAGGTTCTTTTC |
| | (LTR) | TATTTTCCCTAAGCGTCGGCAGGTTTGAGAAATAAAGGG |
| | (ERVK/LTR) | ACAGAGTACAAAAGAGAGAAATTTTAAAGCTGGGCGTC |
| | | CGGGGGAGACATCACATGTTGGTAGGTTCCATGATGCCC |
| | | CACAAGCCGTAAAAACCAGCAAGTTTTTATTAGTGATTT |
| | | TCAAAAGGGGAGGGAGTGTACGAATAGGTGTGGGTCAC |
| | | AGAGATCACGTGCTTCACAAGGTAATAGAATATCACAA |
| | | GGCAAATGGAGGCAGGGCGAGATCACAGGACCACAGGA |
| | | CCGGGGTGAAATTAAAATTGCTAATGAAGTTTTGGGCAC |
| | | CATTGTCATTGATAACATCTTATCAGGAGACAGGGTTTG |
| | | AGAGACAACCGGTCTGACCAAAATTTATTAGGTGGGAA |
| | | TTTCCTCGTCCTAATAAGCCTGGGAGTGCTATGGGAGAC |
| | | TGGGGCTTATTTCATCCCGTAGAGGCAACCATAAAAGAC |
| | | AGGCCCAAGCGGCCATTTAGAGCCTCCCAGGACATTCTC |
| | | TTTCTCAGGGTGTTCCTTGCTGAGAAAAAGAATTCAGAT |
| | | ATTTCTCCATTTGCTTTTGAAAGAAGAGAAATATGGCTC |
| | | TGTTCCCGGCTCACGGCGCAGATTTAAGGTTATCTCCTTT |
| | | TCCCTGAACAATTGCTGTTATCCTGTTCTTTTTTCAAGGT |
| | | GCCCAGATTTCATATTGTTCAAACACACATGCTTACAAT |
| | | TTGTGCAGTTAAGCAATATCACAGGGTCCTGAGGGACAT |
| | | ACATCCTCCTCAGCTGACAGGATTAAGAGATTAAAGTAA |
| | | AGACAGGCATAGGAAATCACAAGGGTATTGATTGGGGA |
| | | AGTGATAAGTGTCCATGAAATCTTCACAATTTATGTTTA |
| | | GAGATTGCAGTAAAGACAGGCATAAGAAATTATAAAAG |
| | | TATTAATTTGGGGAACTAATAAATGTCCATGAAATCTTC |
| | | ACAATCCACGTTCTTCTGCCATGGCTTCAGCCGGTCCCTC |
| | | CGTTCGGGGTCCCTGACTTCCCGCAACA |
| 296 | THE1A | TGATATGGCTTGGCTGTGTCCCCACCCAAATCTCATCTTG |
| | (LTR) | AATTGTACGTTCCATAATTCCCATGTGTTGTGGGAGGGA |
| | | CCCAGTGGGAGATAATTGAATCATGGGGAGCAGTTTCCC |
| | | CCATACTGTTCTCATGGTAGTGAATAAGTCTCATGAGAT |
| | | CTGATGGTTTTATCAGGGGTTTCTACTTTTGCATCTTCCT |
| | | CATTTTCCTCTTGCCACcACCATGTAAGAAGTGCCTTTCA |
| | | CCTCCCACCATGATTCTGAGGCCTCCCCAGCCATGTGGA |
| | | ACTGTAAGTCCAATTAAACTTCTTTTTCTTCCCAGTCTTG |
| | | GGTATATCTTTATCAGCAGCATGAAAATGGACTAATACA |
| 297 | AluY | TTTTTTTTTTTTTTTTTTTTTTTTTTTTTTGAGACGGAGTCT |
| | | CGCTCTGTCGCCCAGGCTGGAGTGCAGTGGCGCGATCTC |
| | | GGCTCACTGCAAGCTCCGCCTCCCGGGTTCACGCCATTC |
| | | TCCTGCCTCAGCCTCCCGAGTAGCTGGGACTACAGGCGC |
| | | CCGCCACCACGCCCGGCTAATTTTTTGTATTTTTAGTAGA |
| | | GACGGGGTTTCACCGTGTTAGCCAGGATGGTCTCGATCT |
| | | CCTGACCTCGTGATCCGCCCACCTCGGCCTCCCAAAGTG |
| | | CTGGGATTACAGGCGTGAGCCACCGCACCCAGCC |
| 298 | AluSx | TTTCTTTACTTCAACCTTCTTTTTTTTTTTGAGACAGAGTC |
| | | TGCTCTCGTTGCCCAGGCTGGAGTGCAATGGCGCAACCT |
| | | CAGCTCACTGCAACCTCTGCCTCCTGGGTTCAAGCAATT |
| | | CTCCTGCCTCAGCCTCCCAAGTAGCTGGGATTACAGGCA |
| | | TGCGCCACCACGCCCAGCTAATTTTGTATTTTTAGTAGA |
| | | GACGGGGTTTCACCATGTTGGCCAGGCTGGTCTTGAACT |
| | | CCTGACCTCAGGTGATCCACCCGCCTCAGCCTCTCAAAG |
| | | TGCTGGGATTACAGGCGTCAGCCACCGCACTCAGCC |
| 299 | AluY | GGCCGGGCGCGGTGGCTCACGCCTGTAATCCCAGCACTT |
| | (FLAM_C) | TGGGAGGCCGAGGCGGGCGGATCACGAGGTCAGGAGAT |
| | (ALU) | CGAGACCATCCTGGCTAACACGGTGAAACCCCGTCTCTA |
| | | CTAAAAATACAAAAAATTAGCCGGGCATGGTGGCAGGC |
| | | ACCTGTAGTCCCAGCTACTCAGGAGGCTGAGGCAGGAG |
| | | AATGGCGTGAACCCAGGAGGCGGAGCTTGCAGTGAGCC |
| | | GAGATCATGCCACTGCACTCCAGCCTGGGTGACAGAGTG |
| | | AGACTCCATCTCAAAAAAATGACACAAAATAAAATAAA |
| | | ATAA |
| 300 | AluSx | GGCCGGGCGTGGTGGCTCATGCCTGTAATCCTAGCACTT |
| | (AluSx1) | TGGGAGGCCGAGGCTAGCGGATCACCTGAGGTCAGGAG |
| | | TTTGAGACCAGCCTAGCCAACATGGCGAAACCCTGTCTC |
| | | TACTAAAAATACAAAAAATGGCTGGGCATGGTGGCAGA |
| | | CATCTGTAATCCCAGCTACTCAGGAGGTTGAGACAGGAG |
| | | AATTGCTTGAACTCAGGAAGCGGAGGTTGCAGTGAGCT |
| | | GAGATTGCGCCACTGCACTCCAGTCTGGGTGACAGAGCA |
| | | AGACTCTGTCTCAAAAAAATAAAATAAAATAAAAATAA |
| | | AATA |
| 301 | AluY | GGCCGGGCGTGGTGGCTCATGCCTGTAATCCTAGCACTT |
| | (AluSx1) | TGGGAGGCCGAGGCTAGCGGATCACGAGGTCAGGAGTT |
| | | TGAGACCAGCCTAGCCAACATGGCGAAACCCTGTCTCTA |
| | | CTAAAAATACCAAAAAATGGCTGGGCATGGTGGCAGAC |
| | | ATCTGTAATCCCAGCTACTCAGGAGGTTGAGACAGGAG |
| | | AATTGCTTGAACTCAGGAAGCGGAGGTTGCAGTGAGCT |
| | | GAGATTGCGCCACTGCACTCCAGTCTGGGTGACAGAGCA |
| | | AGACTCTGTCTCAAAAAAATAAAATAAAATAAAAATAA |
| | | AATA |
| 302 | ALR/Alpha | TTCTTCTCACGGAGTGAAACTTTTCCTTTGATTGAGCAGT |
| | (Satellite/centr) | TTCGAAACTCTCTTTTTGTAAAATCTGCAAGTGAACATTT |
| | | GGAGTGCTTTGTGGCCTATGATAGAAAAGGAAATATTTT |
| | | CACTTAAAATCTAGACAGAAGCAATCTGAGAAACTTCTT |
| | | TGTGATGTTTGTATTCATCTTACATAGTTAAACCTTTCTT |
| | | TTGATTGAGCAGTTTTGAAACTCTCTTTTTGTCGAATCTG |
| | | TAAGAGGACATTTGGAGTGCTTTCAGGCCTATGGTGAAA |
| | | AAGGAAGTATCTTCTCATGAAAACTAGACAGAAGAATT |
| | | CAGACAAACTTCTTTGTGATGTGTGCATTTATCTCACAG |
| | | ACTTGAACCTTTCTTTTGATTGAGCAGTTTGAAACACTCT |
| | | TTTTGTAGAATCTGCAAGTGGACATTTGGAGCACGTGGA |
| | | GGCCTATAGTGGAAATGGAAATGTCTTCACACTAAAAAC |
| | | TAGACAGAAGAATTCTGAGAAACTTCTTTGTGATCTTTG |
| | | CATTCATCTCACAGAGTTGAACCTTTCTTTTCATTGAGCA |
| | | GTTTGAAAACACTCTTTTTGTAGAATCGGCAAGTGGACA |
| | | TTTGGAACGCATTGCAGACTATGATGGAAAAGGAAATA |
| | | TCTCCA |
| 303 | Alu | CAGCGCAAGTAATATATCCTGGAATTGGAAGCAGAATG |
| | (SINE/Alu) | GGAACTATGGACTCAGTGAAGATTGTTCTGGACATTATT |
| | | TGATTCTTCTTTCTCGCTGCCAATTGGATACTTTTTTTTTT |
| | | TTTTTGAGACAGAGTCT |
| 304 | Alu | CAGCGCAAGTAATATATCCTGGAATTGGAAGCAGAATG |
| | (SINE/Alu) | GGAACTATGGACTCAGTGAAGATTGTTCTGGACATTATT |
| | | TGATTCTTCTTTCTCGCTGCCAATTGGATACTTTTTTTTTT |
| | | TTTTGAGACAGAGTCT |
| 305 | ALR/Alpha | TTCATCTTACAGAGATAAACATTTATCTTGATTCAGCAG |
| | (Satellite/centr) | TTTGGAAACACTGTTTTTATCCATTCTGTGAATGGACATT |
| | | TGGGAGTTCATTAAGGCACACATGAAAAGGTGTATATCC |
| | | CAGGGTAAAAACTAGAAGGAAGCTATCTGAGAAACCAC |
| | | TCTGTGGTGTGTGCATTGATCTCGCTGATTTAAAGCTTTC |
| | | TTTTGATTCAGCAGTTTGGAATCACTGTTTGTGCTCATTC |
| | | TGTAAATGGATATTTGGGAGCTCATTGAGGTCAATGGCA |
| | | AAAAAGTGAATAACTGAGGATAAAAACTAGAAATAAGC |
| | | TATCTGAGAAACGGATTTATGATGTGTGCATTCATCTCG |
| | | CAAAATTAAACATTTCTTTTCCTTCAACAGTTTGGAAAC |
| | | ACGGTTTTTATAGGATCTGCAAAGGGATATTTTGGAGCA |
| | | CATTGAGGTCTATGGTGTAAAAGAAAATATCTTCAGATA |
| | | AAACCTAGAAAGAAGCTTTCTGAGAAATTGCATTTTGAT |
| | | GTGTGCATTCATCTCACAGAGTTAATACTTTTTTTTGATT |
| | | CACCAGTGTGGATACACTGTTTTTGTCCATTCTGCTAATG |
| | | GACCTTTGGGGGCTCATTTAGGCCAATGGCAAAAAAGTG |
| | | AATATCCCAGGATAAAAACTAGAAGGAAGGTATCTGAG |
| | | AAACTGCTATGTGATGTGTGCCATCATCTCACAGTCTTA |
| | | AAAGTTTCTTTTCATTCACCAGTTTGGAAACAGTGTTTTT |
| | | GTAGAATCTGCAAAGTCATATTTTTGAGT |
| 306 | ALR/Alpha | GATGAATGCACATATCACAAAAAGTTTCTCAGATTGCTT |
| | (Satellite/centr) | CTGTCTAGATTTTATGTGAAGTTATTTCCTTTTCTACCAT |
| | | AGAACCCAAAGCACTTATAATGTCCCCTTGCAGATTCTA |
| | | CAAAAAGAATGTTTCAAAACTGCTCAATCAAAAGAAAG |
| | | GTTCAAATTTGTGAGATGAATGCTCGCATCACCAAGAAG |
| | | TTTCTCAAAATACTTCTGTCTAGTTTTTATGTGAAGACAT |
| | | TTCTTTTTCCGTGATAGGTCTCAAAATGATCAAAATTTCC |
| | | ACTTGCAAATTCTACAAAAAGAGAGTTTCAAAGCTGCTC |
| | | AATCAAAAGAAAGGTTTATATCTGTGAGATGAACACAC |
| | | ACTTCACAAAGCAGTTTCTAAGATTTCTTCTGCCTAGATT |
| | | TTATGTAATGATATTTCCTTTTCTACCATACTTTGCAAAC |
| | | TGCTCCAAATGTCCACCTGTAGATTCTACAAAAACAGTG |
| | | TTTCCAAACTGCTCAATCAAAAGAAAGGTTAACTCTGTG |
| | | AGATGAACTCACACATCACAAAGAAGTTTCTCAGAATTC |
| | | TTCTGTCTAGTTCTAATGTGAAGATATTTCCTTTTGGACC |
| | | AGAGGCCCCAAGGCTATCGAAATGTCCCCTTGTAGATTC |
| | | TACAAAAAGAGTATTTCAAAACTGGTCCATCAAGAGAA |
| | | AGGCTCAACTCTGGGAGATGAATGCACACATCACAAAG |
| | | AAGTTTCTCAGAATGCTTCTATCTAGTTTTTACATGAAGC |
| | | TATTTCCTTTTCCACCATAAGCCTCAAAGCGCTCCAAAT |
| | | GTCCACTTGCAGGTTCTGCAAAAAGAGAGTTTCCAAAGT |
| | | TCTCAATCAAAAGAAACGGTTAACTCTGTGAGATAAATG |
| | | CACACATCACAAAGAAGTTTCTCAGAATTTTTCTGTCTA |
| | | GTTTTTGTGTGAAGATATTTCATTTTC |

TABLE 2 provides sequences of illustrative polynucleotides that can be used in a synthetic delivery system disclosed herein, for example, for delivery to cancer cells, such as pancreatic cancer cells.

TABLE 2

| SEQ ID NO | Description | Sequence |
|---|---|---|
| 307 | AluSx | AGCCAGGTGCAGTGGCTCACACCTGTAATCCCAGCACTT |
| | (AluSx1) | TGGGAAGCTGAGGCGGGTGGATCACTTGAGGTCAGGAG |
| | | TTCGAGACCAGCCTGGCCAACATGGTGAAACCCCGTCTC |
| | | TACTAAAAAAGTAAAAATTAGCCAGGTGTGGTGGCGGG |
| | | CACCTGTAATCCCAGCTACTCAGGAGGCTGAGGCAGGA |
| | | GAATTGCTTGAACCCGGGAGGCGGAGGTTGCAGTGAGC |
| | | TGAGATTACACCACTGCACTCCAGCCTGGGCAAGAGAG |
| | | CGAGAATCCGTCTCAAAAAAAAAAAAACAAAAAAAACA |
| | | AAAAAC |
| 308 | MIRc | AATTACAACAACCCTTAATATTTGTAAAGCGCTTGATAA |
| | (MIRB) | TTCAGGGAGGGCTTTTAAAAACTTCTATCCATCTCTTCCC |
| | (MIR) | CCCAGCAATCCTATGAGGTAGGCAGGGCTGGAATCATTT |
| | | GCTGCATTTTATCGAATAGGAAGCCATGACTCGTAGAGG |
| | | TCTGGTGACTTAACTGAGGTCACCCACTGAGGACGTGGC |
| | | AGAGCTGGGTCCTGAATCTGTGGCCCTCAGGCTCTTTGC |
| | | CTGGCCCTGCCTCACATAGACTACGCTGCCTCT |
| 309 | AluJb | TTTCTTACTTTTTTTATTTATTTTTTTCTTGAGACAGGGTC |
| | (AluSx1) | TCACTTGGTCACCCACGCTGAAGTGCAGTGGCgTGATCT |
| | | TAGCCCACTGCAACCTCTACCTCCCGGGCTCAAGcGATC |
| | | CTCCCTCCTCAGCCTCCTGAGTAGCTGGGACCACAGGCA |
| | | TCTGCCACCACACCCAGCTAATTTTTGTATTTTTAGTAGA |
| | | GATGGGGTTTCACCATGTTGGTCAGGCTGGTCTCAAACT |
| | | CCTGgCTTCAaGTGATCTGCCCACCTCAGCCTCCCAAAGT |
| | | GCTGGGATTACAGATGTGAGCCACTGCGCCTGGCC |
| 310 | AluSx1 | TTGATTTTTTTTTCTTTTATCTTTTTTTTTGAGACAGAGTT |
| | (AluSq) | TTGCTCTGTTGCCCAGGCTGGAGTGCAGTGGCGTGATCT |
| | (ALU) | TGGCTCACTGCAGTGTCCGCCTCCTGGGTTCAAGCGATT |
| | | CTCCTGCCTCAGCCTCCTGAGTAGCTGGGATTACAGGCA |
| | | TGCACCACCACGCCCGGCTTCATTTTTTATTTTTAGTAGA |
| | | GACAGGGTTTCACCATGTTGGTCAGGCTGGTCTCGAACT |
| | | CCTGACCTCAGGTGATCCACCCGCCTTGGCCTCCCAAAG |
| | | TGCTGGGATTACAGGCGTGAGCCACCGTGCCCGGCC |
| 311 | AluSx | GGCTGGGCATGGTGGTTCACGCCTATAATCCCAGCACTT |
| | (AluSp) | TGGGAGGCCGAGGCAGGCAGATCACCTGAGGTCGGGAG |
| | (Alu) | TTCGAGACCAGCCTGACCAATATGGTGAAACCCCGTCTC |
| | | TACTAAAAATACAAAAATTAGCCGGGCGTGGTGGTGCA |
| | | TGCCTGTAATCCCAGCTACTTGGGAGGCTGAGGCAGGAG |
| | | AATCGCTTGAACCCAGGAGGCGGAGGTTGCAGTGAGCC |
| | | GAGATTGCGCCACTGCACTCCAGCCTGGGTAACAGAGC |
| | | GAAACTCCGTCTCAGAAAAAAAAAAAGGCAGATATGAA |
| | | AAAGG |
| 312 | MTL2B4 | AATATTAATTTGGGCTCTCCAGAGAAACAGAACCAATAG |
| | (MTL2B4) | ATTACACATAGATATATAGAAAGACAGTTATTACAAGG |
| | (ERVL) | AATTGGCTCACAGGATTATGTAGGCTGAGAAGTCCCATG |
| | | ACCTGCCGTCTGCAAGCTGGAGACCCAGGAAAGCCGGT |
| | | GGTGTAGTTCCAGTCCGAGTTGGGAGGTCTGAGAATCGG |
| | | GAGAGCTGACAGAGTAAGCTCTAGTTGCAGCCTGGAGT |
| | | CCAGAGGGCAAAGAACACCTATGTCCCATCTCAAGCAC |
| | | GGTCAGGCGTCCCAGCTCAAGCAGAGAGAAAGAAGACT |
| | | TTCCTGCTCAGCCTCCTTGCTCTAAGCAGCCTCCCGACG |
| | | GATTGGATGAAGCTCACCCACGCTGGGGAGGGCAGCCA |
| | | CCTTCACTCAGTCCACCAATTCAAATGCTAATCTCATCC |
| | | AGAAACACCCTCACAGGCACACCCAGAAATAATGTTTA |
| | | GCCACATATCTGGGCATCCTGAGGACCAGTCAAATTGAC |
| | | TCATAAGTCTCACCATCATA |
| 313 | AluSx1 | TTATGACTATTATTTCTTTTTTTTTTTTTTGAGATGGAGTC |
| | | TCGCTCTATTGCCCAGGCTGGAGTGCAGTGGCATGATCT |
| | | CGGCTCACTGCAACCTCTGCCTCCCGGGTTCAAGCGATT |
| | | CTCCTGCCTCAGCCTCCTGAGTAGCTGGGATTACAGGTG |
| | | CCTGCCACCACACCCGGCTAATTTTTGTATTTTTAGTAGA |
| | | GATGGGGTTTCACTGTGTTGGCCAGGATGGTCTTAAACT |
| | | CCTGACCTCAAGTGATCCACCCACCTCGGCCTCCCAAAG |
| | | TGTTGGGATTACAGGCGTGAGCCACCATGCCCGGCC |
| 314 | L2a | CTCCATGTTCTGACCTCAGGTGATCtGCCCgCCTCCGCCT |
| | (L2a/LTR40b/MLT1J2) | CCCATAAGTTCCTTCTGCACCTATTCCATTCCATCTGCAA |
| | | TGTCATCCTGGCCAACCTGTGAATGGGTATAGCAGGATA |
| | | GTAAGTTAGTCCCAGTGTTTAGGCAGACTCCATAGACCA |
| | | GATTTAACGTGTTTGTATAAACCGCATGGCTCGCTCTTA |
| | | CTCAGCCTTCGGGTCGGCCTTAAGATACACAGTTTTAAG |
| | | AAAAGCATTGTCTTCACTCTTAGTGTAGATCAGGTCCTC |
| | | CTCnCCCCCAnCACGTTATGTCCCTGTCATTGAACATTAT |
| | | ACTTTTATGAAGAATTGTGAGCTGTCTGAGATTTTACCTT |
| | | ACTTGCAAaCTAACAAGTGGGCCTGCCAtAGTTTCATGTC |
| | | TGGCAGAAGACTCCAACAGGTCAGAGATTTTCATCTGTC |
| | | TTGTTCACTATTGTCTACATAGTGCCTGGAAAAATGCCT |
| | | GGCACACAGTAAACCCTCAATCAGTATTTTATGAATGAG |
| | | TGAAC |
| 315 | MTL1J2 | TGTGTTAGTCAGGATAGGCTAACCTCTGTAACACACAAC |
| | (parAluSp- | CCCAATTTTTCAGTGGCTCAGAACAATAGGAGTTTCTTT |
| | FULLMTL1J2) | CTTACTCATGTAAAGTCCAGGTGTGGCAGAAACTTGCGG |
| | (LTR) | GGGAGAAAGTCTACTCCACAGCATCACTCAGGGACCCA |
| | | GGCTCCTGCCTTACGGTGGTTCCGCCATTTTCCAGGGCCT |
| | | CGGAGTCCTCCATCCCATCTCCTCCATTCAGCTGGTGGA |
| | | TGTGGGAGGAGAGCGTGTATGAAAGACCACACAGAAGC |
| | | TTTTTTTGGGCCAGGCTTTGAGACGGCGCACATCAGTTT |
| | | CgCTCTTGTTGCCCAGGCTGGAGTGCAATGGcGCAATCTC |
| | | GGCTCACCGCAAAAGACACTGGGAAATTTGGTCTGGCC |
| | | ATGTGTCTAGGAAGCAGAGGAGATGGGGTTTCAtAAACT |
| | | CCTGAACTCTCTCTACCACG |
| 316 | AluSx | GGCCGGGTGCAGTGGCTCATGCCTGTAATCCCAGCACTT |
| | (AluY) | TGGGAGGCCGAGGCGGGTGGATCACCTGAGGTCAGGAG |
| | | ATCAAGACCATCCTGGCTAACACGGTGAAACCCCATCTC |
| | | TACTAAAAATACAAAAATTAGCTGGGCGTGGTGGCAGG |
| | | CGCCTGTAGTCCCAGCTACTCGGGAGGCTGAGGCAGGA |
| | | GAATGGCGTGAACCCAGGAGGCAGAGCTTGCAGTGAGC |
| | | CGAGATTGAGCCACTGCACTCCAGCCTGGGGGACAGAG |
| | | CAAGACTCTGCCTCAAAAAAAAAAAAAAATGGAAAAAA |
| | | AAAAAA |
| 317 | (MIRB) | AAGATGGGGAGGATGGTGGTGGAGCAGGCTGGGGAGAT |
| | (MIRc-part L2) | GAGGAGCTTGAGGGGCCTAGGAGCTAACCACCCATTCA |
| | (MIRc) | CTGTTTATGGGTGGGGTGGCTCCGGGCAGGTGGCCTGAC |
| | | CCCTCTAAACCTCAGTTTCCCCATCTGTAaAATGGgAGTA |
| | | aTTGTACCTGCCTCACGGGCATGTAGCAGGTGGGAGAGA |
| | | GAGTAATACGGGCAAGACGTCTAGTGCaGTGGCTGGAAC |
| | | ATAACAAGCACCCAGCAAGTGGTGGCCCCATTGCT |
| 318 | AluSx | GGCCGGGCACGGTGGCTCAAACCTATAATCCCAGCACTT |
| | AluSq | TAGAAGGCCAAGGCGGGCAGATCACCTGAGGTCAGGAG |
| | | TTCAAGACCAGCCTGGCCAACATGGTGAAACCCCGTCTC |
| | | TACTAAAAATACAAAAATTAGCCAGGTGTGGTGGCGGG |
| | | CACCTGTAATCCCAGCTACTCGGGAGGCTGAGGCAGGA |
| | | GAATCACTTGAACCCAGGAGGCGGAGGTTGCAGTGAGC |
| | | CGAGATCATGCCACTGCACTCCAGCCTGGGCAACAGAGT |
| | | GAAACTCTGTCTCAAAAAAAAAAAAAAAAAAAAAAAAA |
| | | AAAAA |
| 319 | MamR4 | AAATCGAATTCTCTGTCTCCAATTGCTTCTTGCTTTCAAG |
| | | GGACCCAAGCAGGAGGCTGCTCCGCCACATATAAATGT |
| | | AGGACCTGAAGAAGCAGTCTGCAGCTCAAGGCTGGAAT |
| | | TGGCCTAGATCCTCCCTGGAGGCCCAGATTCACAGTGTA |
| | | ATCGTTCCAAGCCATTAATCACAGGCACCCGGCGGGACT |
| | | GTGTCTCGTCCCAAAGGGCATCTCAAGAGGTTTTGCAGA |
| | | GTTTCATGTAAATGATCATTACACTACTAATAAATATGG |
| | | TTAGTGACTTTGAGCCACATCAGGTGCCTTGGGCCAAGG |
| | | AGCTGGCGGAAGGAGTATCTAGGCTTCCTTAAGTGAAG |
| | | GCACTGAACAGCCAGCAGAATTAATCCATCAACTGCTCT |
| | | CCTTTCTTCCCCACTTCTCATACACACACACACGCACAC |
| | | GCGCGCATGTACACACACGCTGGCGAGAGTTTTAGACCA |
| | | GAGCACCAAGCCCTCCAATGCCCATGTTAAGAAAATATG |
| | | AAGTTAATATGCTCGGCTTCCTGTTGAAGTCCGCCTCATT |
| | | CCCACTAACTGATCAGGATACACTCACAGTTCACCCGCA |
| | | AGAGCAGACAACAAATGAGGAGAGATAGGGCAACGGC |
| | | ACCTCTGTCTGCAGCATTTTTAAAAATATAGATACCTAC |
| | | TCTTTCAAACAGATTTGAAGTGAGAATATGGTTTCTATG |
| | | ATAATACCCAAAAATGAACTGCATAAACCATTGCCTAAT |
| | | GCTGAACAGCACTCCTCGATTCTTCCTAAATCTATACTC |
| | | GCAGTTCTTATGCAAGAAAAAGGTAACAACATCCCTGTT |
| | | CTCCATGCTTCCATTTTACTTCACATCTGGACTGTCACTG |
| | | CAAACAGCTGAATCTGATTTTAAAAAGGTACACATATAC |
| | | CACTTTCATTATCAATGTCTTATACAAATTACCTGATACA |
| | | TGTAGTTACTTAGAAATTATTTCCCATTGCTTCAATATCT |
| | | ATGAACAAAAAGTAAACACTACATAAGATAAAATAAAC |
| | | CAAAATATTAATGAGGCTCACAGAACTACTGATGAATA |
| | | AGCTCAGAAAACTGTTAGTATTTTTTATACTATGTGAAA |
| | | CCAAAGACCGATCAAGAAAATGTATTTACTAACTGTATT |
| | | TAATAACCACTGCAATAAGACTGAATGACTTAAGCAAA |
| | | GATAAATTGATAAAAGGGCCGGGCACGGTGGCTCAaaCC |
| | | TATAATCCCAGCACTTTaGAATGGCCaAGGCGGGCaGATC |
| | | ACCTGAGGTCAGGAGTTCaAGACCAGCCTGGCCAACATG |
| | | GTGAAACCCCGTCTCTACTAAAAATACAAAAAAAATTA |
| | | GCCAGGtGTGGTGGCGGTGCaCCTGTAaTCCCAGCTACTT |
| | | CgGGAGGCTGATCAAGTGGCAGGAGAATCACTTGAACCC |
| | | aGGAGGCGGAGGTTGCAGTGAGCCGAGAACTGCACTCC |
| | | AGCCTGGGCAACAAGAGTGAaACTCTGTCTCAAAAAAAA |
| | | AAAAAAAAAAAAAAAActgaagcaatcaaagcggctagtAAAAGAC |
| | | TTACAGAAAAAGTTGATTTGTAATAGAGGGTCAATCTGG |
| | | CACAACTGTAGATTACCTATAAATGTTTAGAATTATCCT |
| | | CTCTTCTAAAATGCAAACAGAATCAGCATGCAAAATAAC |
| | | TTTGGCTTACATGATTCAATTCTAGTCATCTAACAAAAG |
| | | AATTTACTCCAAATCCGGGAAGTTATGGCCAGCCTGGAG |
| | | GGCTAAGAAGAATCTGAAATAGAAAGAAATTTTTATTAT |
| | | TTAAATACTTCCCCTCTCTTCTGCTAAACAATCTCTTTAG |
| | | GCTTGATGGGCAAAAAGGATGTCGCAAGTTTCTACATTC |
| | | TTCTAAAAAACTCAGTACTTTAAAAGCTGATAGAGAAAT |
| | | TCAGTCACCCAAAGGAGTCAGCTGTCACAGAAAATGTTA |
| | | TCTAATAGTGGAAAGATTACATAATGCCGATCTGCACTC |
| | | GTATCTAATAGAGATTTTGAAGAACTAAGGAATTAGGCC |
| | | TCCTAAGATAAAATCTTTGCAGCATGCAATGAGCAAGTA |
| | | TAAAGATCTCATTCTCTTCCCAGGTGACTGGACAAGGGA |
| | | AAGATTCAAGCAAGCCTCTGGGAAGCGGGGAATATTTC |
| | | AAGGAGACTTACTTATTTCAACAAACATTTACTTGGGAC |
| | | TGGACCATCTATGCTGGGCCTAGGACTGTGCTAGGAGCT |
| | | GATGATACATGGATGAACAGGATTAACATCATATAAGA |
| | | AATGATTTGTTCTACGTGGACGTACTGAATCTCTAAGCC |
| | | AACACTAAAATCAAATTTCATATACTGCCAAAAATCACC |
| | | AAGAACTTTAGTGATGATATACAAATGGAGTTTGATATG |
| | | GAAAACTGGAGGCCAATAAAAATGAGGAAAGAACAACT |
| | | TCCTATAACAGATTAGTTCCCTAGTAGGAAAAACCATGT |
| | | TATTAAAGCTCTAAGTTAGGGTAACATTTAGGAGTATCT |
| | | AGGCTTCCTTGAGCCAAAACAGGTTAATCTCATCAGTTT |
| | | TAGATCATAACTTAAGAAAATATGAAGTTAATATGCTTA |
| | | GAAAGTACTAAAACCACACATTAAAAGAGGCAATAAGA |
| | | CCAAGGCAATAAATTTACTTGTAGTACCGGTTCCGAGAC |
| | | ACTTATTTGGTATTAGAAAGCCATCTAATAAAGGTTAAA |
| | | ATATTAGAGAACATAAACTATAAATGTCAATGACACAA |
| | | AATCTAATCTATCATCTTAAAGCTGCAATCGAGAGGATC |
| | | ACTCTTACAAATGATAGAATTAAAAAGAGGGTAATTAG |
| | | CATCAAAAATTCATTTTTTTTCCTGAATTTAAAACTATAG |
| | | GGCTATTTTTACAGCAAATCAAATGTAGAGCGGTGAAAA |
| | | ATAGGCCCCAAAACCGATCnCAGTGTTATGCCAGCAGAA |
| | | CCTGAAGAAACACATCATGGTCCTCTGGTAAGTAAGCTG |
| | | ATGACCTAACAGCAAATTCAGACACAGAAACAAAAGGA |
| | | ACTTTCAGGTGTCTACACACATATCATTAAGAAAGATTA |
| | | TGTGGCAGATGCTACATCACATTTACGTCTCAGGAAAGA |
| | | CCATGTGGCCCAGAGATGAGGGATGGTTTTTATGTCAAT |
| | | TCAAGTAAAAGTGGTTGCCCCTAAAAATAACCACATCTG |
| | | TATACATAAAGAAAGCTTGTCTGATGATACATGCAGAAG |
| | | ATGAGAAAAGGCTTAAGTGGAAAATGAATATAAAATTG |
| | | GATAATGTGTCACCTTGGCTGACATGGATTACACCAACA |
| | | AGCATAGTAATTCTGCTAAGATTACATACCAATAATTAG |
| | | CTTTCCAGTGTGGGACTGATAATGGATTAACATAGTTCC |
| | | ATTAACGTTAACTCAAGATTATTTTAGAAAATAACTGTA |
| | | AACGCCTGCTCAGATTCAGCCATATAACAGGATACTAGC |
| | | AAGTTCTTACTAACAGACCAGACATTCTGCTGCTTGATT |
| | | CATCTTTTAATTTGGTAGAACTTACTAAAATAACCTTTTC |
| | | TCTTAAATGGCACATCCAAAAACCCTCACAGAAAAAAA |
| | | TaTTTAAAAGTATGTAATCCCCAGTGAGATTCAAAATATT |
| | | GTGGAGATAATAACCAAACAGAACAAATGCACCAACCA |
| | | TCTCGGCGATCACAATAAACACAAGATTAAAAATCAAA |
| | | AACATAACTTTGCAGTAACCTTGTGACTAGCTGGTATCA |
| | | GAGCAGCACGCAAATCTGACAGAAAACAGCAATTAAAC |
| | | AAAGGCATTTCTGTCTTGACAACAGACTTCTATAAAACA |
| | | AGGAACTAAGGGCCGTTTTGGTAAGAAATAGAAAGGCC |
| | | AGATTAATCAGAGGAATGAAATTAATATTTA |
| 320 | AluSx | ACAGTGGCACACGCCTGTAGTCCCAGCACCTTTGGAGGC |
| | (SINE/Alu) | TGAGGTGGGTGGATCACCTGAGTCCAGGAGTTCGAGAC |
| | | CAGCCTGGGCAACATGGCATGACCCTGTCTCTGCAAAAA |
| | | ATATGAAAATTAGCCAGGCATGGTGGTGCGTGCCTGTAG |
| | | TCCTAGCTACTTGGGAGGCTGAAATAGGAGGATCGCTTG |
| | | AGCCCAAGAGTTGAAGGTTGCTGTGAGCCAAGATCGCA |
| | | CCACTACAATCCAGCTTGGGTGTCAGAGTAAGACCCTGT |
| | | CTCAAAACAAAAAGATAAAT |
| 321 | MIR | GTATTTAGATTCCCAGACTTTGGAGCCAGATCGGCTGAG |
| | (SINE/MIR) | TTCAAACCCTGTCTGTGCAACTATCAGCTGTTCAGCTGT |
| | | GTGACCTTGGGCAAGTTACTTAACCTCTCTGAACCCTGA |
| | | TTCCTCCTGCAAACGGTGGGAAAAATAATAGAACCAATC |
| | | TCTCTGGGTTGCTGTAAGGATTAAATTAATTTACAGTGC |
| | | ATGTAACATGCTTACAGCAATGTCTGGCACACAGTAAGC |
| | | ACTAAGTATTTGTTGTTATTTGTCATC |
| 322 | L1M1 | AGGATTCATCACCTTCTGACTTAGGAGCTGCTGGTCTCT |
| | (LINE/L1) | GAATAACCTACAGTGGTACCCCAATAACACATCATGAAC |
| | | ATTGGACTCCGAGATGTGCTGGCTTCAGGTGTGACCCAG |
| | | AACATTTCCCACTGTGGTGGGTATAGTGAAAGACACCTT |
| | | CTGTTTGAGCAAAGCAGAGGAAAAAGTAAATGGGATTC |
| | | TGTCTTGCACCTGAGGTACCAGCTTGGCCACAGTGGGGT |
| | | AGAGCACCAAGCAGACTCTTAGGGTCCCTGAGTCAGCAT |
| | | TTCTGGACCTGCCCTGCACCAAAGAGTAAGTCCCAGTTG |
| | | TGGAAGCAGTCATCATAAGCTGACTGAAGAGTGCTTGG |
| | | GTGTTGAGTGAACATCAGTGATAGCCCAGTGGAAACCCC |
| | | TGTGGGCTGGTGGTGGTGGCAGACACATTGTGAGGCTCC |
| | | TCTGCCTGCAGAAAGGGGAGGGAGGAGTGGTAAGGATG |
| | | TTGTCTTGTAATTTGAATGCCAGCTTAGCTGCAGTAGAA |
| | | TAGAACACGAGATAAATAAGGCTTCTGACTTCAATACCT |
| | | GGCTCCCACACAATATCTCTGAACCTACCTGGGGCCAAA |
| | | GGGAACTCACTGCCCTGAGGAGACACAAACCTGGCTGG |
| | | TTACCACCTGCTGATTGTAGAGGCCTAAGGTCTTGAGTA |
| | | AACTTAGGTGGTAGCCAGGCAGTAATTACACTGGGCCTT |
| | | GGGTGACACCCAGTGCTGTGCTCACTTCAAGTCTAAATT |
| | | AGCACAGTCCCAGTGGTGGGGGCAACAGGGAAGCTTAT |
| | | GTCATCCCACCCCAGCTCCAGGTAGCTCAGCATAGAGAA |
| | | AGAAATTCCATTCGTCTGGAAGAAACTAAGGGAAGAGA |
| | | ACAAGAGTCTCTGCTTGGTAATCCAGATAATTCATCGAT |
| | | TTTAACCAAGACCAACCACATGGTACCTCTATGAGTCTG |
| | | CAAGAACAACGTTATTGAGTTTGAGATCCAAGTACCTTC |
| | | AAATTCTGGAAAGCCTTCCCAACAAGGGTGGACACAAA |
| | | GCCCAGACTGCGAAGGCTACAATAAATGCCTAACTCTTC |
| | | AATGCCAAGACACAGATGAACATCTACACATTAAGACC |
| | | ATCCAGTAAAATATGGCCTCACCTAACAAACTGAATAAG |
| | | GCATAAGAGGCCAATCCTGGAGAAACAAAATAAGTAAC |
| | | TTTTCAGACAGATGATTCAACATAGTTGTGTTGAGGAAA |
| | | CAAATTCAAGATAAAATAGAGAAGGAATTCAGATTCTA |
| | | TCAGATAAATCAAACAAAGAGGTTGAAATAATTAAAAG |
| | | GAATCAAGCAGAAATTGAAGTTAAAAAAGCAATTGGCA |
| | | TACTGAAGAATGCATCAGTCTCTTAATAGCAAAACTGAT |
| | | CAGGCAAAAGAAAAAATTATTGAGCTTGAACATAGGCA |
| | | ATTTGAAAATATGTAGAGAAGACAAAAAAGAATGAAGT |
| | | ATGCCTACAAGATTTAGAAAACACCCTGAAAAGGGCAA |
| | | ATCTAAGAGTTGTTAGCCTTAAAGAGAGAAAGAGAAAG |
| | | GGGTAGAAAGTTTCAAGAATACGATCAGAGAACTTCCC |
| | | AAATCTATAAAGATATCAATATCCAAGTACAACCAGGTT |
| | | ACAGAACACAAAGCAGATTGAACCCAAAGACGACTACC |
| | | TCAAGACATTTAATAATCAAACTCCCAAAAATTAAGAAT |
| | | AAAGGATCCTAAAAGCAGCAAGAAAAAAGAAACAGCAT |
| | | ACAATGAAACTCCAGTACGTCTGGCAGCAGATTTTCAGT |
| | | GGAAATCTTACAGGCCAGGAGAGAATAGCATGACATAT |
| | | TTAAAGTGCTGAAGTAGCAAAGCTTTTACACTAGAATAG |
| | | TCTATCCCGTGAAAATATTCCATAGCCATGAAGGAGAAA |
| | | AAAATGACCTTCCCAGACAAACACTGAGAGATTTCATTA |
| | | AAACCAGACCTGTCCTACAAGTAATACTAAGGGGCATTC |
| | | TCTTCCATCTGTAAGAAAAGAATGTTAATGAACAATAAG |
| | | AAGTCATCTGAAGGTACAGAACTCATTGGTAATAGCACA |
| | | GAGAAAAACTGAGTATTATAATATGGTAACTGTGGTGTG |
| | | TAAAATACTCAAGTAGAAATACTCAATGATGACTCAATA |
| | | CAACTACAACAATGTTTCAAGACAAAGACAGTACAATA |
| | | AGACATAAAGAGAACCAACAAAAAGTTGAAAAATGGGG |
| | | AAGCAAAGTTAAAGGTAGAGTTTTTACTAGTTTTCTTATT |
| | | GCTTATTTGTTCACTTAGGCAATCAGTGTGAAGTTGTCA |
| | | GCAGTTAAAACAATAGGTTATAAGATAGTATTTGTAAGT |
| | | TTCATGGTAACCTCAAATAAAAAAACATACAACAGACA |
| | | CAGAAACCAAAAAGCAAGAAATTAAATCATACCACCAA |
| | | GAAAAATCATGTTCACTAAAATGAAGACAGAAAGGAAG |
| | | GACAAAAGAAAGAGAAGATGCAAAAACAAATAACAAA |
| | | ATGGCAGGAGTGAACACATACTTATCAATAATAATGCTG |
| | | AATGGAAATGCACTAAACACTCCAATTAGATGACATAG |
| | | AATGGGTGAAAAAAAGAAAAAGGAAGACCCAATTATCT |
| | | GCTGCCCACAAGAAATACACTTCATCTATAAAGATACAC |
| | | ATAGACTGAAAAATAAAAGAATGGAAAAATGTTCCATG |
| | | CCAATGAAAACAAAAAAAAAGAACCAAAGTAGCTATAC |
| | | TTACATCAGATAAAATATATTTCAAGATAAAAACTGTGA |
| | | AGACAGAACAAATGTCATTTTATGATAAAGGGATCAATT |
| | | CAGTCAGAGAATATAACAATTTTAAATATCCATGCACCC |
| | | TACACTAAAGCACTGAGACATAGAGTATTAGAGCTAAA |
| | | GAGAGAGGCCCCAATACAATAATAATTGAGACTTCAAC |
| | | ACACCACTTTCAGCATTGTACAGATCTTACAGATAGAAA |
| | | ATCATGAAAGAAACATCAGACTTAATCTGTGCTATAAAC |
| | | CAAATATACCTAATAAATATTTGCAGAACATTTTATCTA |
| | | ATGGCTACAGAACACACATTCTTCTCAGCACATAGATAA |
| | | TTTTCAGATACACAACATTTTAGGTTACAAAACAAGTCT |
| | | TAAAACATTCAGAAAAGTGAAATAATATCAAACATCTCT |
| | | GCCCACAATGGAATATAACTAGAAATCAGTAACAAGAA |
| | | TTTTGGAAACTATACAAACAAATGTAAATTAAACAATAC |
| | | GCTCCTCAATGAAAATTGGATCAGTAAAGAAATTAAGA |
| | | ACGAAATTGAAAAATTTCTTCAAACAAATAATGAAAAA |
| | | ACACATACCAAAACCTATGGGATACAGCAAAAGCAGTA |
| | | CTGAGAAATGTATAGATATATGTGCTTACATCAAAAAAG |
| | | AAAAAAATCATATAACCTAATGTTGCATCTTAAAGAAAA |
| | | AACAGCAAAACAAAAATTATAATAAACATCACAGCAGA |
| | | AATAAAATTCAAATAAAAAATACAAAAAAAATCAATGA |
| | | AATGAAAAGTTGATTTTTTTAAGCAGATAAATAAAATTT |
| | | ACAAACCTTTAGCTTGACTAAGAAAAAAAGAAGACCCA |
| | | CATAAAACCAGAGATGAAAAAGGAGACATTATAACTGA |
| | | CACTGCAGAAATTCAAAAGATTATTAGTGGCTATTATTA |
| | | ACAACTGTATGCCAACTAATTGGAAAATCTAACAAAAAT |
| | | GGGTAAGTTCCCAGATATATACAACTTACCAAGATTAAA |
| | | CAACGAAGAAATCCAAAACATGAACAGACCAATAAGAA |
| | | GTAACAAGTTCAAAGCCATAATAAAAAGTCTCCCAGTA |
| | | AAGAAAAGCCCAGGACCTGATGGCTTCACCACTAAATTC |
| | | TACCCAACATTTAAAGATCTAATACCAGCTCTACTCAAA |
| | | AACTTCCAAAACACAGGAGGAAGGAATACTTCCCAACT |
| | | CATTCTTTGAGGCCAGTATTACCATAATACCCAAATCAG |
| | | AAAAAGATACATCAAAAGATGAAAACTGTACGCCAATA |
| | | TCTATAATAAATATTTATGTAAAAATAACAAAGTGTTAG |
| | | CAAACAGAATTCAACAACACATTAAAAGATTGTTCATTA |
| | | TGACAAAGTGGGATTTATCCCAGGGATGCAAGAATGGTT |
| | | CATCATATGCAATCAATCAGTATGATACATCAACAGAAT |
| | | GAAGGACAAAATCATATGATCATTTCAACTGCTGCTGAA |
| | | AAAAGGATTTAATAAAATTCAACATCCTTTGATAATAAA |
| | | AACTCTCAAAAAGCTGGGTATAGAAAGAACATACCTCA |
| | | AAATAATAAAAACCATATATGACAGATCCACAGCTAGT |
| | | ATCATCCTGAATGGAGGAAAAAAATCTGAAAGTCTTTTC |
| | | TCTAAGATCTGGAACATAACAAGGATGTCCACTTTCAAC |
| | | ACTGTTATTCAACATAGTACTGGAAGTGCTAGCTACAGC |
| | | AGCAGTAAAACGAGAAAAAACTAAATGGCATCCAAATT |
| | | GGAAAGGAAGTCGTCAAATTATTCTTGTTTGCAGATGGT |
| | | ACAGTCTTATATTAAGAAAAATCTAAAGACTCCACCAAA |
| | | AAACAGTAAGAACTGATAAAGTTGCAGGATATAAAATG |
| | | AACATACAAACATCAATAGCATTTCTATATGCCAACAGA |
| | | GAATCTGAAAAAAGAGATTAGAAAGTAATTCCATTTAC |
| | | AGTAGCCACAAATAAAATTAAATATCTAGACATTAACTT |
| | | AACCAAAGAAGTGAAAGATCTGTACAATGAAAACTATA |
| | | AAACATGAATGAAAGACATTGAAGAGGATGGCTGGG |
| 323 | LIMEg | CCCTTGAACTTATTAAAATCTTTTATAATGTAGTACATTT |
| | (LINE/L1) | ACTGACTGCTTTCAGGCAGGCATGAGGACCTTTCACTCC |
| | | ATTTGCTCCATTGTCCCTTTTGAAGGATTCTTGTCATATA |
| | | TATAAAACCTCTTAAGACATAATGTTATTGTTTTATATAC |
| | | TCTTTAATCACCTACATCTTTGCACTTTAATATTTCCTAA |
| | | ATTTGCATTCTTCTCTCTGGGACTAGAAGACCTCAGCTTA |
| | | GCGGTTCTTTTCATGTGGGCCTGAGAAATTTTCTCACTTT |
| | | TTGTTTCACTTTCATTCTTGAAGGAACGTTTCACTGGTTA |
| | | TAGGATGCAAGGTTAGCGGTTATTCTCTTCTGCATCTTCC |
| | | CACTGCATTGTCTTCTGAATCTTATAATTTATGTAAAAAC |
| | | ATTAGTTGTCTGTCTTCCCATTGCTTCTGTGAAAATAATG |
| | | CATGTTTTCCTCTGGTTGTTTTTGACATTTTCTTTGTGTCT |
| | | GTTTCTCAGCAATTTTTCCATGATATATGCTCGGAGCGG |
| | | GCTTACTTATCCTACACCCTGAATCTGTAGCTTGACGTCT |
| | | TTTTGTCAACTTTGGAAAGTTTTTGGCATTATCTCATCAA |
| | | ATATTTCCCCTGTCCTGTTCTTGCTCTTCTCTTGCTGGAG |
| | | TGCAGTCGTTCCTGTATTAGACCTTTGCATCATGTTCTAG |
| | | GTATTTTTATCT |
| 324 | AluSx | GCGGTGGCTCACGCCTGTAATCCCAGCACTTTGGGAGGC |
| | (SINE/Alu) | TGAGGCGGGTGGATCACTTGAGGTCAGGAGTTCGAGAC |
| | | CAGCCTGGCCAACATGGAGAAACCCCATCTCTACTAAAA |
| | | ATACAAAAATTAGCTGGGCGTGGTGGCTCATGCCTGTAA |
| | | TCCCAGCTACTTGGGAGGCTGAGACAGGAGAATTGCTTG |
| | | AACCCAGGAGGTGGAGGTTGCAGTGAGCCAAGAACACA |
| | | CTATTGCACTGCAGCCTGGGTGACAGAGCCAGACTCTGC |
| | | CTGAAAAGAAAAGAAAAGAAAAAGAATATG |
| 325 | AluSz6 | TTTTTCTTTGAGGTAGAGTCTCACTCTGTCACCTAGGCTG |
| | (SINE/Alu) | GAGTACAGTGGTGCAATCTCAGCTCACTGCAACCTCTAC |
| | | TTCCCAGGTTCAAGTGATTTTCGTGCCTCAGCCACCCAA |
| | | GTAGCTGGGATTATAGCCATGCGCCACCACACACAGCTA |
| | | ATTTTTTTGTATTTTTTAGTAGAGATGGGGTTCGCCATGT |
| | | TGGCTAGGCTGGTCTCAAACTCCTGGCCTTAAATGATCC |
| | | ACCCACCTCAGCCTCCCAAATTGCTGGGATTGCAAGCAT |
| | | GAGCCATCGCACCTGGCGCAGAAC |
| 326 | ALR/Alpha | TCAACTCACAGAGTTGAACAATCCTTTTCATACAGCAGT |
| | (Satellite/centr) | TTTGAATCTCTCTTTTTGTAGAATCTCCAATGGACATTTG |
| | | GAGCACTTTGGGGCCTTCGCTCGAAACGGGAATATCTTC |
| | | CCATAAAAACTGGACAGAAGCTTTATCAGAAACTACTTT |
| | | GGGATATGTGCTTTCAACTCACATAGTTGAACCCTTCTTT |
| | | TGATAGAGCAGTTTTGAAACACTCTCATTGTAGAATCTG |
| | | CAAGTGGACATCTGGTGTGCTTTGAGGCCTATGGAGAAA |
| | | AAGGAAATATCTTCACATAAAAACTAGACAGAAGCATT |
| | | CTCAGAAACTTATTTTTGATGTGTGTACTCAACTGACTCA |
| | | GTTACACCTTTCTTTTGATAGAGCAGTTTTGAAACACTCT |
| | | TCTTGCAGAATTTATAAGTGGATATTAGGAAAGCTTTGA |
| | | GGCTTTTGTTGGAAACGGGAATGTCTTCACATAAAAACC |
| | | AGACAGAAGCATTCTCAGGAACATCTTTGTTTTACGCGC |
| | | ATTCATCTCACAGACTTGAAACTTTCTTTTGATAGAGCA |
| | | GCTTTGAAACAAACTTTCTGTAAAATTTGCAAGTGGATA |
| | | TTTGGACAGCTTTGAAGCCTTTGCTGGAAACGGGAGTAT |
| | | CTTCACATAAAAACTAGATGGAAGGATTCTCAGACACTT |
| | | CCTTGGGAGGTTTGCATTCAACTCAAAGAGTTGAACATT |
| | | CCTTTTCATAGAGCTGTTTTGAAACACTCTTTTTGTAGAA |
| | | TCTGCTAGTGGACATTTGGAGCAACTTGAGGCCTGTGGT |
| | | GAAAAATGAAATACCTTCGCATAAAAACTAGACAGAAT |
| | | CATTCTCAGAAACTTCTTTGTGATGTGTGTACTCCACTCA |
| | | CAGAGTTGAACATTTCTTTTGATAGAACAGTTTTGAAAC |
| | | ACTCCCTTGTAGAATCTGCAGGTGTATATTTGGACAGCT |
| | | TTGAGTCTTTCATTGGAAACGGGAATATCTTCTCATAAA |
| | | AACTAGACAGAAGCACTCTCAGAAACTTCTCTGTGATGT |
| | | TTGCCTTCAACTCTCAGAGTTGATCATTCTTTTTCATAGA |
| | | GCAGTTTTGAAACACTCTTTTTGTAGAATCTGCAAGCGG |
| | | ACATTTGGAGCACTTTGAGGCCTGTGGTAAAAAAGGAA |
| | | ATATCTTCACATGAAAACTAGACAGGAGCATTCTAAGAA |
| | | ACTTATTTGTGATGTGTACACTAAACTCACAGAGTTGAA |
| | | CTTTTCATTTGATAGAGCAGTTTTGAAACACTATTTTTGT |
| | | GAATCCTGCAAGTGGACATTTGGAGCACTTTGAGGTATT |
| | | CTTTGGAAACGGGAATATCTTCACATAAAAACTAGAAG |
| | | GAAGCATTCTCAGAAACTTCTTTGTGAGGTTTGCATTCA |
| | | ACTCACAGAGTTGAACTTTTCTTTTGATAAAGCAGTTTTG |
| | | AAACACACTCTGTAGAATCTGTTAATGGATATTTGGACA |
| | | GCTTTGAGGTTTTCGTTGGAAACGGGACTATCTTCACAT |
| | | AAAAACTAGACAGAAGCATTCTCAGAAACTTCTTTGCGA |
| | | TGTTTGCATTCAACTCACAGAGTTGAAGATTCCTTTTCAT |
| | | AGAGCAGTTTTGATATGCTCTTTTTGTGGAATCTGCCTGA |
| | | GGACATTCGGAGTGCTTTGAGGCCTAAGGTGAAAAAGG |
| | | AAATATCTTCACATAAAAACTAGACAGAAGCATTCTCAG |
| | | AAACTTCTTTGTGAAGTGTGTACTCAACTCACAGAGTTA |
| | | AACCTTTCTTTGATAGAGCAGTCTTGAAACACACTTCTT |
| | | GTAGAATTTACAAGTGGATATTAAGACAGCTTTCAGGCT |
| | | TCCATTGGAAATCCTGAAGATATTCGCTGGAATATCTTC |
| | | ACATAAATTGTAGACAGAAGCATTCTCAGAAACATCTTT |
| | | GTGATGTGTGCTGTTGGAACTTTCCTTTGATAGAGCAGT |
| | | TTTGAAACACTCTTTTTGTAGAATTTGCAAGTGGATATTT |
| | | GGACAGCTTTGAAGCCTTCCCAGGAAATGAGTTTATCTT |
| | | CACATAAAAACTAGACAGAATCATTCTCAGAAACTTCTT |
| | | TGTGATGTTTGGTTTAACTCACAGAGTTGAACATTCCTTT |
| | | TCATAGAGCAGTTTTGAAAAACGCTTTTTGTAGAATCTG |
| | | CAAGTGGACATTTGCAGCGTTTTGGGACCCATAGTGAAT |
| | | AAGGAAAGATGTTCACATTAAAACTAGATAGAAGCATT |
| | | CTCAGAAACTTCTTTGTGATGTGTGTACTCAACACACAG |
| | | AGTTGAATCTTTGTTTTGACAGAGCAGTTTGAAACCCTTT |
| | | TTTGTATAATCTGCAAGTGGATATTTGGATAGCTTTGAG |
| | | GCTTCCGTCGGAAACGGGAATATCTTCACATAAAAACTA |
| | | GAGAGAAGCATTCCTCAGAAACTTCTTTGTGAAGTTTGC |
| | | CTTCAACTCACAGAGTTGAACATTCCTTTTCATAGAGCA |
| | | GTTTTGAAACACTCTTTTTGTAGAATCTGCAA |
| 327 | L1P1 | CAGGCCTGAGCAATGGCGGGCACCCCTCCCCCAGCCTCG |
| | (LINE/L1) | CTGCCGCCTTGCAGTTTGATCTCAGACTGCTGTGCTAGC |
| | | AATGAGCGAGGCTTCATGGGCATGGTACCCTCCGAGCCA |
| | | GGTGCGGGTTATAATCTCCTGGTGTGCCGTTTGCCAAGA |
| | | CCATTGGAAAAGTGCAGTATTAGGGTGGGAGTGACCCG |
| | | ATTTTCCAGGTGCTGTCTGTCACCCCTTTCTTTGACTAGG |
| | | AAAGGAAATTCCCTGACCCCTTGTACTTCCCAGGTGAGG |
| | | CGATGCCTCGCCCTGCTTCGGCTCATGCTCG |
| 328 | THE1B | CACCCAAGTCACCTCTTGAATGCTTTGCTGCTTAGACATT |
| | (LTR/ERVL-MaLR) | TCTTCTGCCAGATACCCTAAATAATCTCTCTCAAGTTCAA |
| | | AGTTCCACAAATCTCTAGGGCAGGGGCAAAATGCCACC |
| | | AGTCTGTTTGCTAAAACATAACAAGAGTCATCTTTGCTC |
| | | CAGTTCCCAACAAATTCCTTATCTCCATCTGAGACCACC |
| | | TTATCCTGGATTTCATTGTCCATATCATTATCAGCATTTT |
| | | GGTCAAAGCCACTCCACAAGTCCCTAGGAAGTTCCAAAC |
| | | TTTCCCACATTTTCCTGTCTTCTTCTGAGGCCTCCAACCT |
| | | ATTCCACCCTCTGTCTGTTACCCAGTTCCAAAGTTGCTTC |
| | | TACATTTTCAGGTGTCTTTTCAGCAGTGCCACACTCTACT |
| | | GGTACTAATTTAC |
| 329 | AluSg | AGGCCTGGCACAGTGGCTCATGCGTGTAATCCCAACACT |
| | (SINE/Alu) | GCGGGAAGCTGAGGCAGGAGGATCACTTGAGCCCAGGA |
| | | GCTCATGGCTGTAGTGAGCTGTGATTGCACCACTGCACT |
| | | GCACTCCAGCCTGGGCAACAGCAAGACCCTGTCTCCAAA |
| | | AAAAAAAAACAAAACAAATTAAT |
| 330 | HERV9N-int | TTATGGTGGACATCATTGAATAATTCATGGGCTTTTTCCT |
| | (LTR/ERV1) | AATTCTCCTTAGTCCTTCTAGAACACAGGTCAACAGATG |
| | | TTTGTGACTCCAGTCCCTATGATCTGAGTTGAGGTCCCA |
| | | GTGGGGATCCATACTGGGGACAGCTTGCTGACCAGTAG |
| | | GGAATTTTCCCTTTCTTTGGCTGTCATTTTATCATTTACTT |
| | | GACTAAGATGCCAGGTATCTCCAAACTCTTGGGCTGCAG |
| | | CTAAAGCTGCATTCTTTTCATTAAAGGCCAGGGTTTGAT |
| | | CTAACAAAAGCATGACATCTTTCCAAGTGAGATCGAAG |
| | | GTTTCCCCTAGACCCTGTAGGACATCTATGTACCTATCA |
| | | GGATCATCTGAAAACTTTCCCAGGTCTGCCTTGATCTGC |
| | | TTTAAATCAGAGAGGGAGAAGGGGACATGTACCTGGGT |
| | | TGGGCCAAATTCCCCTTCCCCTACAGCTTGAAGGGGACG |
| | | TAACCGATAGCCCGGGGGTTTTTGTGGTCCTTTGGAGAT |
| | | TTCTTTGCTTATTTCCTTCTGGGCAGGGGAGATTAGAGG |
| | | AGGCTTATCATTAATAGGAGGGGGAGCCATAGGGAGGC |
| | | TAGGATATGGGACTAAGCTGAAAGGTCCTCCTGGGGGA |
| | | TGTAAATTGCAAGCTTTGCATAGTCATGTATTCTCCTTCA |
| | | ATGAAAAGAGTTTGGACATAAGGTATTTCACTCCATTTG |
| | | CCTTCCCTCTTACAGAAAAGGTCAAGCTGCAGGATAGTA |
| | | TTGTAATTTATACTTCCCTCAGGTGGCCATTTTTCCCCAT |
| | | CAGAGAGAGAATATTGGGGCCAGGCTGTAGGGCAGAAA |
| | | AAAATGAGCCGCCTCTTTTTCAGGGTTTGTGGGTCAAAT |
| | | TGGTCCCAATGGCTTAGGATGCATTTCAAAGGTGAGCCT |
| | | GTTGATGCCTGAGTGTTTCCCATCTGAAAGACAAAACCA |
| | | CCTGTGGTTTTGGTTTGTTTTGTTTCTCCCCCACCCACAA |
| | | ACCTGCAACGGTCCCTGGACCCTGCTGATCGGAATAGTT |
| | | GTGCTCCCTGACGCAGCAGCAAAAATACTAGTTTTCCTC |
| | | CCAGACCACAAGGGGGACTGAGAAAGGTCGGATTTAGT |
| | | GGCCCTTACCAACGCATTCTCGAAAACCTGCACCCTTGC |
| | | CTGTCCTCCTAGACCACAAAGAGGACCGAGAAAAATCG |
| | | GATTTAGTGGCCCTTACCGACACATTCTCGAAAACCCAT |
| | | TAGAGTCCTAAGCATTCACCTGTTAGTATTGGGACCTTA |
| | | ACCATGTCCTATAAAGATGTTATGCCCCCCAAATGAAGT |
| | | GGAGTGCCATACCCTGAGGGAGGGAAGGGATGTCCAGG |
| | | GTTGGAAGAGTGACACCTTTTGTCCTTACTTATATGAAT |
| | | AGGAAGGATACAATTTCTGAGGCTCCCCATATTCTAGCT |
| | | TCAGGAATAGCTTTTGTTAGGCCTGCTAGTCTGAGGAGG |
| | | GATCCTAAAATTCCAGATAGTCCCCCCTACGATGGGGCT |
| | | TTGGGCAAAAATTACGTCTTTCTGATTGGTGAGCCTGGG |
| | | TGCCTACAGAAGGTAACAGAATCCCAAATTTATACTAGA |
| | | AATCATTCTTATAGGAGAAACTAGAAAAGCACCAGAGA |
| | | CAGGGAGTGGTTTTTAGAAGCAGGACTAGCCTGGAGAA |
| | | GAGAGGCAAGGGGAAGTTTGTCTGGCAGGCGTTAGGAC |
| | | CCAGGGGGCAATGGTCAGGGTAGATAGAATAGATGGGC |
| | | AAGTCTCGCTTGGGCGACATGACTTTGAGAGTTCCACTC |
| | | ATGGCCACAGGGTCAACCAACTTGTTGTTGGGACCCCGG |
| | | AGCTGAATGGCTTTCCTCTCTGTCGACCCTGGGCTCAGC |
| | | CCAGAAGTACAGGAAAAGCGGAAGCTGGTTCCAGGCAA |
| | | ACCAACGCTACCAACTCTGAAGAGTCAGGGATTGTTAGA |
| | | AAGGCCTTTCCCAGAAAGCCTGACACCTGTGTCTTTAGT |
| | | CTGGCAGCTGCGCTAGTAGCTTTTAACTGGCCGACAGGT |
| | | GCCTGGTATTTAGCCCCCGAATTCTAAGGAAAAATAGGA |
| | | AAGAATAGCAAGCGAAAGGGGTCCGATGGTACTCACCA |
| | | CTTGGCAATAGGCGATAGTCTCACTTCTTGGCGATAGGC |
| | | GATGGTCCCATCTGGGTCGCCAAAA |
| 331 | HERV17-int | CTTTAACCTCCTTGTTACGTTTGTCTCTTCCAGAATCGAA |
| | (LTR/ERV1) | GCTGTAAAACTACAAATGGTTCTTCAAATGGAGCCCCAG |
| | | ATGCAGTCCATGACTAAGATCTACCGCGGACCCCTGGAC |
| | | TGGCCTGCTAGCCCATGCTCCAATGTTGATGACATTGAA |
| | | GGCACCCCTCCCAAGGAAATCTCAACTGCACGACCCCTA |
| | | CTATGCCCCAATTCAGCATGAAGCAGTTAAGAGCGGTTG |
| | | TCAGCCAACCTCCCCAACAGCACTTGGGTTTTCCTGTTG |
| | | AGAGGTGGGACTGAGAGA |
| 332 | LTR81B | CAGTCTTTCAGGACCAGAAGAGATGGATTTTAGGTTTTC |
| | (LTR/Gypsy) | CTTCCACTCAATGTATTCTATATGTACCCTAATCAACAA |
| | | ACATGCCTGGTTGTCACAGGTAGAAGGGGTCTCTACTCT |
| | | GAATCATTCAGAGAAGAAATAATCATATTCTTTCGATGC |
| | | TATCACTAACACAGGACCGTGATCCGAGACAACCAGGTT |
| | | CAGTTCTTGATATGTAAAGGAATTTGGGAAAATATCATC |
| | | TCTCACCACAAAATCTGTATTTTTATGTATATTTAAGCAA |
| | | AATCCTGTTTTGGTCATCATTTTCTTTACCTTTCTTCTATA |
| | | TCCATCCTAATCTCCCTTTCAGC |
| 333 | ALR/Alpha | CCTATGTTAGAATAGAAAATGTCTTCACATAAAATCTAG |
| | (Satellite/centr) | ACAGAAGCAATCTGAGATACTTCTTTGTGGTGTGTCCTT |
| | | TCATCTCAAAGAGTTAAAACTTTCTTTTGATTGAGCAGTT |
| | | TTCAAACTCTCTTTTTGTAGAATCTGCAAGTGGACATTTG |
| | | GAGCGCTTTAAGGCCTGTGGTGGAAAAGGAAATATCTTC |
| | | ACCTAAAAACTAGACAGAAGCAATCTGAGAAACTTCTTT |
| | | GTGATGTGTGAATTCATTTCACAGAGTAAAACCTTTCTTT |
| | | TGATGGAGCCGTTTTGAAACTCTCTTTTTGTAGAATCTGC |
| | | AAATGGACATTTGGAGTTCTTTGAGGCCTATGGTGGAAA |
| | | AGGAAATATCTTCACATAAAAACTAGATAGAAG |
| 334 | ALR/Alpha | TCATCTCACAGAGTTAAACCTTTCTTTTGATTGAGCAGTT |
| | (Satellite/centr) | TTGAAACTCTCTTTTTGTAGAATCTACAAGTGGACATTG |
| | | GGAGCTCTTTGAGGCCTATGTTAGAAAAGGAAATATCTT |
| | | CACATAAAACCTAGATAGAGGCAATCTGAGAAACATCT |
| | | TTGTGATGTGTGCATTCATCTCACGGAGTTAAACCTTTCT |
| | | TTTCGTTGAGCAGTTTTTAAACTGTCTTTTTGTAGAATGT |
| | | GCAAGTGGACATTTGGAGCGCTTTTTGTCCTATGGTGGG |
| | | AAAGGAAGTATCTTCACATAAAAACTAGACCGAAGAAT |
| | | TCTGAGAAACTTCTTTGTGACGTGTACGTTCATCTCACTG |
| | | AGTTGAACCTTTCTTTTGATTGAGCAGTTTGGAAACACT |
| | | CTTTTTGTAGAATCTGCAAGTAGACATTTGGAGCGCTTTT |
| | | CAGCCTACGGTAGAAAAGGAAATATCTTCACATCACAA |
| | | AGAGAGAGTTTCAAAACTGCTCTATCAAAAGAAAGGTTT |
| | | AACCCTGTGCGATGAATGCACATATCACAAAGAAGTTTC |
| | | TCAGATTGCTTCTGTCTAGATTTTATGTGAAGATATTCCT |
| | | TTTTCCACCATAGGCCTCAAAGCGCTCCAAATGTCCACT |
| | | TGCAGATTCTACAAAAAGAGTGCTTCCAAACTGCTCGAT |
| | | GAAAAAAATGGTTCAACTCTGTGAGATGAATGCACGCA |
| | | TCACAAAGAAGTTTCTCAGAATTTTTCTATCTAGTATTTA |
| | | TTTGAAGATATTTCATTTTCCACCAT |
| 335 | AluSc8 | TTTCAGATAGAGTCACCCTCTGTCGCCCAGGCTGGAGTG |
| | (SINE/Alu) | CAGTGGCATGATCTTGGCTCACTGCAACCTCCACCTCCC |
| | | AGGTTCAAGCGATTCTCCTGCCTCAGCCTCCTGAGTAGC |
| | | TGGGATTACAGA |
| 336 | LIP3 | GAACTAATTTACACTCCCACCAACAGTGTAAAAGTGTTC |
| | (LINE/L1) | CTATTTTTCCACAACCTCTCCAGCATCTGTTGTTTCCTGA |
| | | CATTTTAATGATTGCCATTCTAACTGGCATGAGATGGTC |
| | | TCATTGTGGTTTTCATTTGCATTTCTCTAATGACCAGTGA |
| | | TGATAAGCATTTTTTCATACATCTCTTGGCTGCGTAAATG |
| | | TCTTCTTTTGAAAAGTGTCTGTTCATATCCTTTGCCCATT |
| | | TTTTGATGGGGTTGTTGCCTTTTTTCTTGTAAATTTCTTTC |
| | | AGTTCTTTGTAGCTTCTGGATATTAGCCCTTTGTCAGATG |
| | | GACAGATTGCAAAAATCTTCTCCCATTCTGTAGGTTGCC |
| | | TGTTCACTCTGATGATGATGATAGTTTCTTTCGCTGTGCA |
| | | GAAGCTCTTTAGTTTAATTAGATCCCATTTGTCAATTTTA |
| | | ACTTTTTTTGCCATTGTTTTTGGTGTTTTAGACATGAAGC |
| | | CTTTTCCCATGCCTATGTCCTGAATGGTATTGTCTAGGTT |
| | | TTCTTCTAGGATTTTTATGGTCCTAGGTCTTATGTTTAAG |
| | | TCTTTGATCAATCTTCAGTTGATTTTTGTATAATGTGTAA |
| | | GGAAGGGGTCCAGTTTCAGTTTTCTGCATATGGCCAGCC |
| | | AGCTTTCCCAACACCATTTATTAAATAGGGAATCCTTTC |
| | | CCCATTTCTTGTGTGTGTCAGGTTTGTCAAAGATCAGAT |
| | | GGCAGTAAATGTGTGGTGTTATTTCTGAGGCCTCTGTTCT |
| | | GTTCCATTGGTCTATATATCTGTTTTGATACCAGTGCCAT |
| | | GCTGTTTTGGTTACTGTAGACTTGTAGTATAGTTTGAAGT |
| | | CAGGTAGCATGATGCCTCCAGCTTTGTTCTTCTTGCCCAG |
| | | GATTTTCTTGGCTATGTGGGCTTTTTTTTGGTTCCATATG |
| | | AAGTTTAAAATAGTTTTTTCCAATTCTGTGAAGAAAGTC |
| | | AGTGGTAGCTGGATGGGGAGAGCATTGAATCTATAAATT |
| | | ACTTTGGGCAGTAAGGCCATTTTCATGATACTGATTCTTC |
| | | CTATCCATGAGCATGCAAGGTTTTTCCATTTGTTTGTGTC |
| | | CTCTTTTATTTCCTTGAGCAGTGGTTTTTGAAGAGGTCCT |
| | | TCATATCCCTTGTAAGTTGTATTCCTAGGTATTTTATTCT |
| | | CTTTGTAGCAATTGTGAATGAGAGTTCACTCATGATTTG |
| | | GCTCTCTGTTGTCTGTTATTGGTGTATAGGAATGCTTGTC |
| | | ATTTTCGCACATTGATTTTGTATCCTGAGACTTTGCTGAA |
| | | GTTGCTTATCAGCTTAAGGAGGTTTTGGGCTGAGACAAT |
| | | GAGGTTTCCTAAATATACAATCATGTCATCTGCAAACAG |
| | | AGACAATTTGACTTCCTCTCTTCCTATCTGAATACCCTGT |
| | | ATTTCTTTCTCTTGCCTGATTGCCCTGGCCAGAACTTCCA |
| | | ATACTATGTTGAATAGGAGTGGTAAGAGAGGGCATCCTT |
| | | GCCTTGTGCTGGTTTTCAAAGGGAATGCTTCCAGTTTTTT |
| | | CCCCCATTCAGTATGATACTGGCTGTGGGTTTGTCATAA |
| | | AGAGCTCTTATTATTTTGAGATATGTTCCATCAATACCTA |
| | | GTTTATTGAGAGTTT |
| 337 | AluYc3 | GCGGTGGCTCACGCTTGTAATCCCAGCACTTTGGGAGGC |
| | (SINE/Alu) | CGAGGTGGGCGGATCACGAGGTCAGGAGATCAAGACCA |
| | | CGGTGAAACCCCATCTCTACTAAAAATATAAAAAATTAG |
| | | CCGGGCGTGGTGGTGGGCGCCTGTAATCCCAGCTACTCA |
| | | GAGAGGCTGAGGCAGGAGAAGGGCGTGAACCCGGGAG |
| | | GCGGAGCTTGTAGTGAGCTGAGATCGCGCCACTGCACTC |
| | | CAGCACCCCAGCCTGGGTGACAGAGCGAGACTCCGTCTC |
| | | AAAAAAATAATAATAATAATTAAATAAATAA |
| 338 | LIPA10 | CCAAGAAGTTCAAACTGGACAGAACCCACCACAGCTCC |
| | (LINE/L1) | ACAAAGCCACTGTAGCCAGACTGCCTCTCTACATTCCTC |
| | | CTCTCTGGGCAGGCCACCTCTGAAAGAAAGGCAGCAGC |
| | | CCCAGTCAGGGGCTTATAGATAAAACTCCCATCTCCCCG |
| | | GGACAGAGCACTTGGGGGAAGGGGCATCTGTGGGTGCA |
| | | GCTTCAGCAGACTTAAACGTTCCTGTCTGCTGGCTCTGA |
| | | AGAGAGCAGCAGATCTCCCAGCACAGCACTCCAGCTCTC |
| | | CTAAGGGACAGACTGCCTCCTCAAGTGTGTCCCTGAACC |
| | | CTGTGCCTCCTGACTGGGAGACACCTCCCAGCAGGGATC |
| | | GACAGACACCTCATACAGGAGAGCTCTGGCTGGCATTTG |
| | | GCGGGTGCCCCTCTGGGACAAAGCTTCCAGAGGAAAGA |
| | | ACAGGCAGCAATCTTTGCTGTTCTCAGCCTCTGCTGGTG |
| | | ATACCCATTTAAACACAGTCTGGAGGGGGCCTCCAGCAA |
| | | ACTCCAGCAGACCTGCAGAGGAGAGGCCTGACTGTTAG |
| | | AAGGAAAACTAACAAACAGAAAGGAATAGCATCAACAT |
| | | CCACAAAAAGGACATCCACACAAAAACCCCATCCAAAG |
| | | GTCACCAACATCAAAGACCAAAGATAGATAAATCCATG |
| | | AAGATGAGGAAAAAACAGCGCAAAAGGGCTGAAAATTC |
| | | CAAAAACCAGAATGCCTCTACTCCTCCCAAGGATCACAA |
| | | CTCCATGCCAGCAAGGGAACAAAACTGGACGGAGAATG |
| | | AATTTGACCAACTGACAGAAGTAGGTTTCAGAAGGTGG |
| | | GTAACAACAAACCCCTCTGAGCTAAAGGAGCATGGTCT |
| | | AAGCCATTGCAAGGAAGCTAAGAACCTTGAAAAAAGAT |
| | | TAGAGGAATTGCTATCTAGAATAACCAATTTAGAGAAG |
| | | AATATAAATGATCTGATGGAGCTGAAAAACACAGCTTG |
| | | AGAACTTTGTGAGGCATACACAAGATTCAATAGCCAAAT |
| | | CGATCAAGCAGAAGAAAGGATATGAGAATTGAAGTTCA |
| | | ACTTAATGAAATAAAATCTGAACATTAAAGTAGAGATA |
| | | AAAGAATGAAAAGGAACAAACAAAGCCTCCAAGAAATA |
| | | TGGGACAATGTGAAAAGACCAAACCTACGTTTCAATGGT |
| | | GTACCTGAAAGTCATGAAGAGAATGGAACCAAGTTGGA |
| | | AAACACTCTTCAGGATATTATCCAGGAAAACTTCCCCAA |
| | | CCTAGCAAGAGAGCCCAACACTAAAATTCAGGATACAC |
| | | AGAGAACACTACAAAGATACTCCTCAAGAAGACCAACC |
| | | CCAAGACACATAATCATCAGATTCACCAAGGTTGAAAG |
| | | GAAGGAAAAAATGTTAAGAGCAGCCAGAGAAAAGGGTC |
| | | AGGTTACCCACAATAGGAAACCCATCAGACTAACAGCT |
| | | GATCTCTCTGCAGAAAACCTACAAGCCAGAAGAGAGTA |
| | | GGGCCAATACTCAACATTCTTAAACAAAAGAATTTTTAA |
| | | CCCAAAATTTCATATCCAGTCAAACTAAGCTTCATAAGC |
| | | AAAGGAGAAATAAAATCTCTTATGGACAAGCAAATGCT |
| | | GAGAGATTTTGTCACCATCAGGCCTGCCTTACAAGAGCT |
| | | CCTGAGGAAGCACTAAATATAAAAAGGAAAAACTGGTA |
| | | CCAACCACTGCAAAAACATACCAAATTGTAAAGACCAT |
| | | CAACACCATGAAGAAATGGCATCAAATAAAGGGCAAAA |
| | | TAACCAACTAACATCATAATGACAGGATCAAATTCACAC |
| | | ATAACAATATTAACCTTAAATGTAAATGGGCTAAATGCC |
| | | CCAATTAAAAAACACAGACTGGCAAATTGGAAAGAGTC |
| | | AAGACAAATTG |
| 339 | AluSz | TTTTTTTTTTCCCAGACAGAGGCTCACTCTGTTGCCCAGG |
| | (SINE/Alu) | CTGGAGTGCAGTGGCATGATCTCGGCTCATTGCAACCTC |
| | | TGCCTTCTAGATTCAAGGGATTCTCCTGCCTCAGCCTCCC |
| | | AAGTAGCTGGGATTACAGGCCCACACCACCACGCCTGG |
| | | CTAATTTTTGTATTTTGAGTAGAGACGGCGTTTCATCATG |
| | | TTGGCTAGGCGGGTCTCGAATTCCTGACCTCAAGTGATC |
| | | TGCCCGTCTCCGTCTACCAAAGTGCTGGGATTACAGGCG |
| | | TGAGCCACCACGCCCGGCCTGAAGCC |
| 340 | REP522 | TAGATGGAGGGGGAAAAACAGGGTGGGGAGCGGGAGG |
| | | GAGGGAAGGTTTTGCAGAAAGACGGCGGTTAAAAAGTT |
| | | TATGGGTAGATGGAGGGGGAAAAGAGGGTGGCAAGTGG |
| | | GAGGAGGAAAGAGGGTGATAAGGGGGGGAAGTGGGGG |
| | | TGAGCAGTAGGGAGAGAAGGTTTTGTGAAAAAAATGGG |
| | | GAGAAAGGTTTTGGTTAGACGGGAGAAAAGAGGGTAAC |
| | | AAGTTGGGGAAGGAAAAAGAGGGTAGCTAGGAAGAAG |
| | | ACAAGGTTTTGCGAAAAGATAGCGGCCAGAAAAGAAAG |
| | | ACGGTGGAGAAAGGAAAGACGGTGGGTAAAAAGTGGGT |
| | | AGATGGAGTGGGGAAAGAGGGT |
| 341 | AluSc8 | TTTCAGATAGAGTCACCCTCTGTCGCCCAGGCTGGAGTG |
| | (SINE/Alu) | CAGTGGCATGATCTTGGCTCACTGCAACCTCCACCTCCC |
| | | AGGTTCAAGCGATTCTCCTGCCTCAGCCTCCTGAGTAGC |
| | | TGGGATTACAGA |
| 342 | L1P3 | ATTCAGAAGACAAATCTCACATGCAAAGACACACATAG |
| | (LINE/L1) | GCTCAAAATAAGTGGATGGAGGAATATTTACCAAGCAA |
| | | ATGGCAAGCAAAAAGAAAAAGCAGGGGTTGCAATCCTA |
| | | GTATCTGATAAAACAGGCTTTAAACCAAGAAAGATCAA |
| | | AAAAGACAAAGAAGGTCATTACATAATGGTAAAGGGAT |
| | | CAATGCAACAAGAAGAGCTAACTATCCTAAATATATAC |
| | | GCACCCAATACAGGAGCACCCAGATTCATAAAGTAAAT |
| | | TCTTAGAGACTACAAAGAGACTTATACTCCCATACAATA |
| | | ATAGTGGGAGATTTTAACACCTCATTGTTGATATTAGAT |
| | | CAATGAGACAGAAAATTAACAAGGATATTCAGGACTTG |
| | | AACTCAGCACTGGACAAAGCAGATTTAATAGACATCTAC |
| | | AGAACTCTCTACCTCAGATCAACAGAATATACATTCTTC |
| | | TCAGCACCACATAGCACTTATTCTAAAATTGACCACATA |
| | | ATTGGAAGTAAAACACTCCTCAACGAATGCAAAAGAAT |
| | | GGAAATCATAACAAACAGTCTCTCAGACCACAGTGCAA |
| | | TCAAATTAGAACTCAGGATTACGAAACTTACTCAAAACT |
| | | GCACAACTACATGGAAACTGAACAACCTGCTCCTGAAC |
| | | GACTACTGGGTAAATAATGAAATGAAGGCAGAAATAAA |
| | | TAAGTTTTTGAAACCAATGAGAACAAAGACACAACATA |
| | | CCAGAATCTCTGGGACACAGCTAAAACAGTG |
| 343 | L2a | TATTTTTAAGCAAAGACTTAGAGAGAGAGAGGAAGTTA |
| | (LINE/L2) | GCTATGCACATTGTGGGTGCGGTGTGGTTAGGGTGGCCC |
| | | TGAGATAGGGATATGCCTGGTGTGTTTACGGAACAGCTA |
| | | AGAGGCCAGGGAACTAGAGGAAATTGAGTGGGAAGGA |
| | | GAAGAGTAGGAGATTAGTTTTGAAAAGCAAATGGTTTG |
| | | GGAATATGGATCATGTAAGAGCACATGAGATTTTATAAG |
| | | GATTTCATTTTACTCAGGGTGAAATGAAGAAGCACTAAA |
| | | A |
| 344 | AluJb | GTTCATGCCTGTAATCCCAGTGCTTAGGGAGGCCGAGGT |
| | (SINE/Alu) | GGGTGGATTGCTTGAGCCCAGGAGTTCGAGACCAGCCTG |
| | | GACAAAATGGCAGAAACTCCATGTCTACAAAAAATACA |
| | | AAAATTAGCCGGGCATGATGTTCTGCGCCTGTAGTCCCA |
| | | GCTACTCAGGAGGCTGAGGTGGGAGGATCGCTTGAGCC |
| | | CGGGAGGCGGAGTTTGCAGTGAGCTGAGATGTCACTGC |
| | | ATTCCAGCCTGGCAGACAGAGCCAGACTCTGTCTCAAAA |
| | | GAAAAAAAGAAAAA |
| 345 | LIPA7 | ATGGAATACTATGTAGCCCTAAAAAAAGATGAGTTCATG |
| | (LINE/L1) | TCCTTTGCAGGGACATGGATGAAGCTGGAAACCATCATT |
| | | CTCAGCAAACTAACACAGGAACAGAAATCCAAACACTG |
| | | CATGTTCTCACTCATAAGTGGGAGTTGAGCAATGAGAAA |
| | | ACATGGACACAGGGAGGGGAACATCACACACTGGGGCC |
| | | TTTTGGGGAGTTGGGGGCTAGGGGAGGGATAACATTAG |
| | | GAGAAATACCTAATGTTATTTCTCCTAATATTATTTCAGC |
| | | CTAATGATGGGTTGATGGGTGCAGCAAACCAGCATGGC |
| | | CCATGTATACCTATGTAACAAACCTGCATGTTCTACATA |
| | | TGTATCACAGAACTTAAAGTATGTATATATAAAAAA |
| 346 | LIPA10 | TTAGTCTGATGGGCTTCCCTTTGTGGGTTACCTGACCTTT |
| | (LINE/L1) | CTCTCTGGCTGCCCTTAACATTTTTTCCTTCATTTCAACTT |
| | | TGGTCAGTCTGACGATTATGTGTCTTGGGGTTGCTCTTCT |
| | | CAAGGAGTATTAGTGGTGTTCTCTGTATTTCCTGAATTTG |
| | | AATATTGGCCTGTCTTGCTACGTTGGGGAGGTCCTCTGG |
| | | ATAATATCCTGAAGAGTGTTTTCCAACTTGATTCCATTCT |
| | | TCCCGTCACTTTCAGGTACACCAATCAGACGTAAGTTTG |
| | | GTCTTTTCACATAGTCCCATATTTCTTGGAGTCCTTGTTC |
| | | ATTCTTTTTCATTCTTTTTATCCCTAATCTTGTCCTGATGC |
| | | TTTATTTCATTAAGTTGATCTTCAATCTCTGATATCCTTT |
| | | CTTCCGCTTGATCGATTCGGCTATTAATACTTGTGTATGC |
| | | TTCACGAAGTTCTCATGCTATGTTTTTCAGCTCCACCAGC |
| | | TCATTTATGTTCTTCTCTAAACTGGTTACTCTAGTTAGCA |
| | | ATTCCTCTAACCTTTTTTCAAGGTTCTTAGCTTCCTTGCA |
| | | TTGGATTAGAACATGCCCCTTTAGCTCAGAGGAGTTTGT |
| | | TATTACCCACCTTCTGAAGCCTACTTCTGTCAATTTGTCA |
| | | AACTCATTCTCTGTCCAGTTTTGTTCCCTTGCTGGCAAGG |
| | | AGTTGTGATCCTTTGTAGAGGAGGTATTCTATTTTTTGGA |
| | | ATTTTCAGCTATTTTGCCCTGGTTTTTCCTCATCTTTGTGG |
| | | ATTTATCTACTTTTGGTCTTTGATGCTGGTGACCTTCGGA |
| | | TGGGGTTTTTGTGTGTGTGTCCCTTTTGTTGATGTTGATG |
| | | CTATTCCTTTCTGTTTGTTAGTTTTCCTTCTAACTGGCCCG |
| | | TCTGGTGCAGGTCTGCTGGAGTTTTCTGGAGGCCCACTC |
| | | CAGACCCTGTTTGCCTGAGTATCACCAGCGGAGCCTGCA |
| | | GGACAGCAAAGATTGCTGCTTGTTCCTTTCTCTGGAAGC |
| | | TTTATCCCAGAGGGGCACCTGCCAGATGGCAGCTGGAGC |
| | | TCTCCTGTATGAGGTGTCTGTTGACCCCTGCTGGGAGGT |
| | | GTCTGCCAGTCAGGAGGCACGGGGTCAGGGACCCACTT |
| | | GAGGAGGCAGTCTGTCCCTTAGCAGAGCTCGAGCACTGT |
| | | GCTGGGAGATCTGCTTCTCTTTTCAGAGCCAGAAGGCAG |
| | | GAATGTTTAAGTCTGTAGAACTGTGCCCACAGCCGCTCC |
| | | TTTCCCCCAGGT |
| 347 | AluSq2 | GCCGAGCGCGGTGGCTCAAGCCTGTAATCCCACCACTTT |
| | (SINE/Alu) | GGGAGGCAAAGGTGGGTGAATCACCTGAGGTCAGGAGT |
| | | TCGAGACCAGCCTGACCAACATGGTGAAACCCCATCTCT |
| | | ACTAAAAATACAAAAATTAGCCAGGCCTGGTGTCGGGT |
| | | GCCTGTAATCCCAGCTACTCAGGAGGCTGAGACAGGAG |
| | | AATCACTTGAACCCAGGAGGTGGAGGTTGCAGTAAGCC |
| | | GAGACCGTGCCCATTGCACTCCAATCTGGGCGACGAGA |
| | | GTGAAACTCCATCTTAAAAAAAAAAAAAAA |
| 348 | L1P3 | GCCATTTTCACGATATTGATTCTTCCTATCCATGAGCACG |
| | (LINE/L1) | GAATGTTTTTCCATTCGTTTCTGTCCTCTCTTATTGCCTTG |
| | | AGCAGTGGTTTGTAGATCTCCTTGAAGAGGTCCTTCACA |
| | | TCCTTTGTAAGTTGGATTCCTAGGTATTTTATTCTCTTTGT |
| | | AGCAATTGTGAATGGGAGTTCACTCATGATTTCA |
| 349 | LTR41C | CCTTAATAGTTAATCTCCTATTAATAAGTAAGTGATTCTT |
| | (LTR/ERVL) | TAATTAAACTTTCCTCATTCAAATGGCCATATGCTTTCTG |
| | | TCTCTCGACTAGATGCTGAGTGATATACCCCCCA |

### Cargo

In some embodiments, the present disclosure provides compositions and methods for cell-, tissue-, and/or organ-specific binding, targeting, uptake, nuclear localization and/or genomic integration of a cargo. A synthetic delivery system disclosed herein can be used to deliver a cargo to a target cell. For example, a recognition sequence of a synthetic delivery system can bind to a receptor disclosed herein, thereby facilitating binding, uptake, nuclear localization, and/or genomic integration of the cargo of the synthetic delivery system.

A cargo can be delivered to a membrane or surface of a target cell. A cargo can be delivered to the cytoplasm of a target cell. A cargo can be delivered to the cytosol of a target cell. A cargo can be delivered to the nucleus of a target cell.

A cargo can be or can comprise, consist essentially of, or consist of a nucleic acid cargo. A cargo can be or can comprise a DNA, such as a dsDNA or a ssDNA. A cargo can be, comprise, or encode an RNA, such as an mRNA, rRNA, tRNA, siRNA, shRNA, or ncRNA. A cargo can be or can comprise a transgene.

A cargo can be or can comprise a nucleic acid that encodes a protein. A cargo can be or can comprise a nucleic acid that encodes a therapeutic protein. A cargo can be or can comprise a nucleic acid that encodes, for example, a cytotoxic polypeptide (e.g., a caspase, such as a self-activating, inducible, or non-inducible caspase, a protein that induces p53-mediated apoptotic cell killing CASP3, CASP8, CASP9, BAX, DNA fragmentation factor (DFF) 40, or Herpes Simplex Virus Thymidine Kinase (HSV-TK)), a tumor suppressor, an antigenic peptide, an antibody or antigen-binding fragment thereof, an enzyme, a cytokine, a signaling molecule, a microbial (e.g., bacterial or viral) antigen or epitope, a polypeptide that increases an immune response, or a polypeptide that reduces an immune response. A cargo can be or can comprise a nucleic acid that encodes a diagnostic protein. A cargo gene that encodes a protein can be operatively linked to and/or under regulatory control of a promoter, such as a promoter that is also part of the cargo. The promoter can be, for example, constitutive, inducible, and/or cell type-specific for expression of an RNA and/or protein in the target cell. A cargo can be, comprise, or encode an immunomodulatory factor. A cargo can be, comprise, or encode an immune checkpoint modulator, such as an immune checkpoint inhibitor or activator. A cargo can be or can comprise a diagnostic protein. A cargo can be, comprise, or encode a one or more neoantigens or tumor associated antigens. A cargo can be, comprise, or encode a factor that alters sensitivity of a target cell to a drug. A cargo can be a nucleic acid that encodes an immune receptor, such as a chimeric antigen receptor (CAR). A cargo can be or can comprise a nucleic acid that does not encode a protein.

In some embodiments, a cargo encodes an endogenous protein or functional RNA molecule that is either lacking or functionally impaired in a subject. In some embodiments, a subject carries a mutation in a gene, such as cancerous mutation, a loss-of function mutation, or a gain-of-function mutation that can be treated by supplying a therapeutic cargo, for example, a transgene encoding the lacking or functionally impaired protein or RNA, or to inhibit a pathogenic protein or RNA.

In some embodiments, a cargo is a nucleic acid that inserts into the genome and causes genetic or genomic instability and cell death (e.g., of a cancer target cell).

In some embodiments, a cargo is a nucleic acid that includes a natural or unnatural nucleotide analogue or base.

A cargo can be or can comprise a polypeptide. The polypeptide can be a therapeutic polypeptide. The polypeptide can be a diagnostic polypeptide. The polypeptide can be an anti-cancer polypeptide. The polypeptide can be a cytotoxic peptide, for example, inducing cell death, of the target cell, such as apoptosis, necroptosis, or necrosis. A cargo can be or can comprise, for example, a tumor suppressor, an antigenic peptide, an antibody or antigen-binding fragment thereof, an enzyme, a cytokine, a signaling molecule, a polypeptide that increases an immune response, or a polypeptide that reduces an immune response.

A cargo can be or can comprise a small or organic molecule (e.g., small molecule therapeutic or fluorescent dye). The small or organic molecule can be a therapeutic agent. The small or organic molecule can be a diagnostic agent. The small or organic molecule can be a cytotoxic agent. The small or organic molecule can be an anti-cancer drug. The small or organic molecule can be a kinase inhibitor.

In some embodiments, a cargo can be a dye (e.g., a fluorescent dye), a radioactive isotope, or a contrast agent for use in diagnostic imaging in vivo.

In some embodiments, a cargo is a detectable label (e.g., a fluorescent label, quantum dot, a Quasar Dye, or a radioactive label). A detectable label can be a radioactive label (e.g., a radioactive isotope) that facilitates the visualization of distribution of the herein disclosed constructs via positron emission tomography (PET) or single photon emission computed tomography (SPECT). A detectable moiety can comprise a radioactive isotope of at least one element.

A cargo can be or can comprise a radionucleotide. A cargo can be or can comprise a polymer. A cargo can be or can comprise a nanoparticle. In some embodiments a cargo can cause DNA damage either from outside the target cell or from within the target cell.

A cargo can be covalently or noncovalently attached to a polynucleotide or synthetic delivery system.

In some embodiments, a synthetic delivery system does not include a cargo. For example, in some embodiments a polynucleotide disclosed herein can directly exert an effect on a target cell, e.g., by inducing cell death after binding, uptake, nuclear trafficking, and/or genomic integration of the polynucleotide. In some embodiments, a synthetic delivery system acts as an antagonist to reduce uptake of ctDNA.

### Pharmaceutical Compositions

Compositions disclosed herein can be pharmaceutical compositions (e.g., formulations), and methods can utilize pharmaceutical compositions. For example, in some embodiments provided is a pharmaceutical composition comprising a compound, such as a synthetic delivery system, and a pharmaceutically-acceptable excipient, vehicle, carrier, or diluent. In some embodiments provided is a pharmaceutical composition comprising an antagonist compound (e.g., an antagonist of a receptor associated with uptake of ctDNA) and a pharmaceutically-acceptable excipient, vehicle, carrier, or diluent.

An active compound (e.g., therapeutic agent, synthetic delivery system, or antagonist) can be combined with other chemical components, such as carriers, stabilizers, diluents, dispersing agents, suspending agents, thickening agents, and/or excipients. The pharmaceutical composition can facilitate stability of the synthetic delivery system or antagonist, and administration to an organism.

Pharmaceutical formulations for administration can include aqueous solutions of the active compounds in water soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. The active ingredient can be in powder form for constitution with a suitable vehicle, for example, sterile pyrogen-free water, before use.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers, and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

The disclosed compositions can optionally comprise pharmaceutically-acceptable preservatives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Non-limiting examples of pharmaceutically-acceptable carriers include saline, Ringer's solution, and dextrose solution. In some embodiments, the pH of the solution can be from about 5 to about 8, from about 7 to 8, or from about 7 to about 7.5. Further carriers include sustained release preparations such as semipermeable matrices of solid hydrophobic polymers containing the compound. The matrices can be in the form of shaped articles, for example, films, liposomes, microparticles, or microcapsules.

A pharmaceutical composition described herein can be in a unit dosage form suitable for a single administration of a precise dosage. In unit dosage form, the formulation can be divided into unit doses containing appropriate quantities of one or more synthetic delivery systems, antagonists, or therapeutic agents. The unit dosage can be in the form of a package containing discrete quantities of the formulation. Non-limiting examples are packaged injectables, vials, and ampoules. An aqueous suspension composition disclosed herein can be packaged in a single-dose non-reclosable container. Multiple-dose reclosable containers can be used, for example, in combination with or without a preservative. A formulation for injection disclosed herein can be present in a unit dosage form, for example, in ampoules, or in multi dose containers with a preservative.

### Methods

Reducing Uptake of ctDNA

The transfer of ctDNA to target cells has been associated with transmission of genetic information between tumor cells, transfer of cancer-associated (e.g., oncogenic) mutations between cells, and induction or transfer of resistance of a cancer to anti-cancer therapeutic agents, such as chemotherapeutic drugs. Accordingly, compositions and methods that reduce the uptake of ctDNA by target cells can be useful.

An antagonist can be used to reduce uptake of ctDNA by target cells by interfering with the interaction between ctDNA and a receptor. The antagonist can be or comprise, for example, a receptor antagonist that binds to the receptor and blocks or reduces binding of ctDNA to the receptor, and/or blocks cellular uptake of the ctDNA mediated by or associated with the receptor. The antagonist can bind to and antagonize, for example, an armadillo-like protein, an APP (amyloid precursor protein) family member, a p120(ctn)/plakophilin subfamily member, an MHC protein, a GPCR, an adhesion protein, a tyrosine phosphatase, an integrin, an ion channel, a mineral transporter, a nutrient transporter, or an ATPase. In some embodiments, the antagonist binds to and antagonizes APLP2, PKP4, HLA-A, CD41, CD97, ITGB5, ITGA2B, Integrin αVβ5, PI4K2A, TMEM120A, PTK7A, Ceruloplasmin, PTPRF, SLC16A1, SLC7A5, ATP1B1, ATP2B4, PIP4K2A, or P142A.

In some embodiments, the antagonist binds to and antagonizes APLP2. The antagonist can be an anti-APLP2 antibody or antigen-binding fragment thereof, for example, an antibody of clone JE54-62, clone 4B5, clone EPR5938(2), clone 408813, clone 25A47, clone 11n11, clone JE54-62, clone 6H54, clone C4, clone DS203AB, clone 6D0.4, clone FQS6049(3), clone 397702, or comprising one or more CDRs therefrom.

In some embodiments, the antagonist binds to and antagonizes PKP4. The antagonist can be an anti-PKP4 antibody or antigen-binding fragment thereof, for example, an antibody of clone 433.10.3, clone 1B2, clone 406.3.1, clone 7.7.9, clone 1B11, clone 4H7, clone EML786, clone 518, or comprising one or more CDRs therefrom.

In some embodiments, the antagonist binds to and antagonizes HLA-A. The antagonist can be an anti-HLA-A antibody or antigen-binding fragment thereof, for example, an antibody of clone R0.87, clone 7G7F9, clone 3H2205, clone BB7.2, clone OTI2D11, clone OTI3H6, clone LBI4D11, clone LBI2D11, or comprising one or more CDRs therefrom.

In some embodiments, the antagonist binds to and antagonizes CD41. The antagonist can be an anti-CD41 antibody or antigen-binding fragment thereof, for example, an antibody of clone 9B10, clone HIP8, clone EPR4330, clone PM6/248, clone RKCD41, clone 7E3 (Abciximab), clone BAG 75E5, clone MEM-06, clone JM11-19, clone TBP8, or comprising one or more CDRs therefrom.

In some embodiments, the antagonist binds to and antagonizes CD97. The antagonist can be an anti-CD97 antibody or antigen-binding fragment thereof, for example, an antibody of clone VIM3b, clone MEM-180, clone 03, clone 3F10C2, clone 7, clone 3A7, clone 380905, clone 024, clone 6D7E6, clone EPR4428, clone EPR4427, clone 4G39, or comprising one or more CDRs therefrom.

In some embodiments, the antagonist binds to and antagonizes Integrin αVβ5. The antagonist can be an anti-Integrin αVβ5 antibody, an anti-ITGB5 antibody, an anti-ITGA2B antibody, or antigen-binding fragment thereof, for example, an antibody of clone ABGC-9, clone 06, clone 34D7, clone 708N2, clone 4G7, clone ABGC-9, clone 13C2, clone NKI-M7, clone 6, clone 23C6, clone 2C4, clone 900, clone 2B5A7, clone 1D8, clone 900, clone 2Q1009, clone 9E2, clone 36A4, or comprising one or more CDRs therefrom.

In some embodiments, the antagonist binds to and antagonizes PI4K2A. The antagonist can be an anti-PI4K2A antibody or antigen-binding fragment thereof, for example, an antibody of clone 4F2, clone 3E1, clone B-5, or comprising one or more CDRs therefrom.

In some embodiments, the antagonist binds to and antagonizes TMEM120A. The antagonist can be an anti-TMEM120A antibody or antigen-binding fragment thereof, for example, comprising one or more CDRs therefrom.

In some embodiments, the antagonist binds to and antagonizes PTK7. The antagonist can be an anti-PTK7 antibody or antigen-binding fragment thereof, for example, an antibody of clone 4F9, clone 2E7, clone OTI2E7, clone 525222, clone 4C6, clone 4A6, clone 188B, clone OTI1G4, clone 4D4, clone 1A8, clone 4F9A1, or comprising one or more CDRs therefrom.

In some embodiments, the antagonist binds to and antagonizes PTPRF. The antagonist can be an anti-PTPRF antibody or antigen-binding fragment thereof, for example, an antibody of clone S165-38, clone 6D7C7, clone W7C6, clone 3B1, clone N165/38, clone N165/43, clone E6W4X, clone E8W3H, or comprising one or more CDRs therefrom.

In some embodiments, the antagonist binds to and antagonizes Ceruloplasmin. The antagonist can be an anti-Ceruloplasmin antibody or antigen-binding fragment thereof, for example, an antibody of clone 3C8A5, clone FQTJTS7, clone 3B11, clone ARC5018-06-01, clone 2, clone C1, clone EPSISR6-12, clone EPSISR6-42, clone H-3, clone 6C3K9, or comprising one or more CDRs therefrom.

In some embodiments, the antagonist binds to and antagonizes SLC16A1. The antagonist can be an anti-SLC16A1 antibody or antigen-binding fragment thereof, for example, an antibody of clone 9D12, clone 20S76, clone 882616, clone 14612, clone 70-E-5, clone 35C7, clone FSS24817(C), clone GT14612, or comprising one or more CDRs therefrom.

In some embodiments, the antagonist binds to and antagonizes SLC7A5. The antagonist can be an anti-SLC7A5 antibody or antigen-binding fragment thereof, for example, an antibody of clone EPR17573, clone BU53, clone MEM-108, clone 4D9, clone IPO-T10, clone EP3-1, clone 2G5H3, clone 5E0, clone UM7F8, clone REA387, or comprising one or more CDRs therefrom.

In some embodiments, the antagonist binds to and antagonizes ATP1B1. The antagonist can be an anti-ATP1B1 antibody or antigen-binding fragment thereof, for example, an antibody of clone 464.8 (8A Beta), clone 23A21, clone 4M5T3, clone M17-P5-F11, clone 464.8, clone ARC1232, clone 16C6, clone EPR12195, or comprising one or more CDRs therefrom.

In some embodiments, the antagonist binds to and antagonizes ATP2B4. The antagonist can be an anti-ATP2B4 antibody or antigen-binding fragment thereof, for example, an antibody of clone 2C7, clone 2G8, clone 41C7, clone KBO, clone JA9, clone 5F10, clone D1, clone H8, or comprising one or more CDRs therefrom.

In some embodiments, the antagonist binds to and antagonizes PIP4K2A. The antagonist can be an anti-PIP4K2A antibody or antigen-binding fragment thereof, for example, an antibody of clone 3D3, clone 3A3, clone OTI1B2, clone OTI3D3, clone 3A9, clone D83C1, clone 3H5, clone EML620, clone DS2718AB, clone LBIlB2, or comprising one or more CDRs therefrom.

In some embodiments, the antagonist binds to and antagonizes P142A. The antagonist can be an anti-P142A antibody or antigen-binding fragment thereof, for example, comprising one or more CDRs therefrom.

In some embodiments, the antagonist is or comprises a ctDNA antagonist that binds to the ctDNA and thereby reduces binding of ctDNA to the receptor, and/or blocks cellular uptake of the ctDNA mediated by the receptor. The antagonist can bind to, for example, a target sequence in a ctDNA, polynucleotide, synthetic delivery system, or transposable element disclosed herein. The antagonist can bind to a recognition sequence in the ctDNA.

An antagonist can be or can comprise a protein, polypeptide, or peptide.

An antagonist can be synthetic, artificial, engineered, or non-naturally occurring, e.g., within a subject.

An antagonist can be or can comprise an antibody or an antigen-binding fragment thereof. The antibody or antigen-binding fragment thereof can be or can comprise any antibody class, isotype, and/or suitable fragment, including those disclosed herein. The antibody or antigen-binding fragment can be monoclonal. The antibody or antigen-binding fragment can be polyclonal. The antibody or antigen-binding fragment can be recombinant. The antibody or antigen-binding fragment can be artificial, engineered, or non-naturally occurring.

An antibody can be described with reference to the basic four chain antibody unit, which comprises two heavy chain (H) polypeptide sequences and two light chain (L) polypeptide sequences. Each of the heavy chains can comprise one N-terminal variable (VH) domain and three or four C-terminal constant domains (CH1, CH2, and CH3, and in some cases CH4). Each of the light chains can comprise one N-terminal variable (VL) domain and one C-terminal constant (CL) domain. The light chain variable domain is aligned with the heavy chain variable domain and the light chain constant domain is aligned with heavy chain constant domain CH1. Each light chain is linked to a heavy chain by one covalent disulfide bond. The two heavy chains are linked to each other by one or more disulfide bonds depending on the heavy chain isotype. Each heavy and light chain also comprises regularly-spaced intrachain disulfide bridges. The C-terminal constant domains of the heavy chains (e.g., CH2 and CH3, or CH2, CH3, and CH4) comprise the Fc region, Fc domain, or Fc fragment of the antibody, which can mediate effector functions, for example, through interactions with Fc receptors or complement proteins.

The light chain can be designated kappa or lambda based on the amino acid sequence of the constant region. The heavy chain can be designated alpha, delta, epsilon, gamma, or mu based on the amino acid sequence of the constant region. Antibodies can be categorized into five immunoglobulin classes, or isotypes, based on the heavy chain. IgA comprises alpha heavy chains, IgD comprises delta heavy chains, IgE comprises epsilon heavy chains, IgG comprises gamma heavy chains, and IgM comprises mu heavy chains. Antibodies of the IgG, IgD, and IgE classes comprise monomers of the four chain unit described above (two heavy and two light chains), while the IgM and IgA classes can comprise multimers of the four chain unit. The alpha and gamma classes are further divided into subclasses on the basis of differences in the sequence and function of the heavy chain constant region. Subclasses of IgA and IgG expressed by humans include IgG1, IgG2, IgG3, IgG4, IgA1, and IgA2.

An antagonist disclosed herein can comprise, for example, an IgG, IgG1, IgG2, IgG3, IgG4, IgA, IgA1, IgA2, IgD, IgE, or IgM class antibody, or a fragment (e.g., Fc region) thereof.

Generally the constant regions of an antibody can mediate various effector functions, while the variable regions primarily mediate antigen binding. Different IgG isotypes or subclasses can be associated with different effector functions or therapeutic characteristics, for example, because of interactions with different Fc receptors and/or complement proteins. Antibodies comprising constant domains or Fc regions that engage activating Fc receptors can, for example, participate in antibody-dependent cell-mediated cytotoxicity (ADCC), antibody-dependent cellular phagocytosis (ADCP), complement-dependent cytotoxicity (CDC), induction of signaling through immunoreceptor tyrosine-based activation motifs (ITAMs), and induction of cytokine secretion. Antibodies comprising Fc regions that engage inhibitory Fc receptors can, for example, induce signaling through immunoreceptor tyrosine-based inhibitory motifs (ITIMs).

Different antibody subclasses comprise varying abilities to elicit immune effector functions, which can be utilized or modified in antagonists disclosed herein. For example, wild type IgG1 and IgG3 can effectively recruit complement to activate CDC, and IgG2 elicits minimal ADCC. IgG4 has a lesser ability to trigger immune effector functions and can be used, e.g., where reduced immune effector functions are desired.

The variable (V) regions of an antibody or antigen-binding fragment thereof can mediate antigen binding and define the specificity of a particular antibody for an antigen. The variable region comprises relatively invariant sequences called framework regions, and hypervariable regions, which differ considerably in sequence among antibodies of different binding specificities. The variable region of each antibody heavy or light chain comprises four framework regions separated by three hypervariable regions. The variable regions of heavy and light chains fold in a manner that brings the hypervariable regions together in close proximity to create an antigen binding site. The four framework regions largely adopt an f3-sheet configuration, while the three hypervariable regions form loops connecting, and in some cases forming part of, the f3-sheet structure.

Within hypervariable regions are amino acid residues that primarily determine the binding specificity of the antibody. Sequences comprising these residues are known as complementarity determining regions (CDRs). One antigen binding site of an antibody can comprise six CDRs, three in the hypervariable regions of the light chain, and three in the hypervariable regions of the heavy chain. The CDRs in the light chain are designated L1, L2, and L3, while the CDRs in the heavy chain are designated H1, H2, and H3. CDRs can also be designated LCDR1, LCDR2, LCDR3, HCDR1, HCDR2, and HCDR3, respectively. The contribution of each CDR to antigen binding varies among antibodies. For example, in some embodiments HCDR3 primarily determines antigen specificity, and one or more other CDRs can be dispensable or specificity can be maintained despite mutations in the other CDRs. CDRs can vary in length. For example, CDRs are often 5 to 14 residues in length, but CDRs as short as 0 residues or as long as 25 residues or longer exist. Several methods are used to predict or designate CDR sequences.

Certain antibodies or antigen-binding domains contain less than six CDRs. For example, certain antibodies lack a light chain, and can be referred to as heavy chain only antibodies (HCAbs). HCAbs have three CDRs in a variable region referred to as VHH. A single domain antibody, or nanobody, can be generated from such a VHH region of a heavy chain only antibody.

An antagonist disclosed herein can comprise a suitable set of one or more CDRs (e.g., six CDRs or three CDRs) to bind to a receptor and antagonize it. The CDRs can be as identified by, for example, the Kabat, Chothia, IMGT, paratome, or AHo method. Single domain antibody CDRs can be identified, for example, using the single domain antibody database (SAbDab), based on common sequence elements, or based on a sequence alignment to the Chothia numbering scheme.

In some embodiments, an antagonist is or comprises an antigen-binding fragment. An antigen-binding fragment can be, for example, a Fab, Fab′, F(ab′)2, dimer or trimer of Fab conjugates, Fv, scFv, minibody, dia-, tria-, and tetrabody, nanobody, VHH, or linear antibody.

In some embodiments, an antagonist is or comprises an antigen-binding fragment of an ankyrin protein, ankyrin repeat protein, designed ankyrin repeat protein (DARPin), affibody, avimer, adnectin, anticalin, Fynomer, Kunitz domain, knottin, or β-hairpin mimetic.

An antagonist can be or can comprise a ligand of the receptor associated with uptake of ctDNA or a derivative of the ligand, for example, a peptide ligand, a peptide mimetic ligand, a protein or polypeptide ligand, a chemical ligand, or a small molecule ligand.

An antagonist can be or can comprise a receptor, one or more domains thereof, and/or a soluble form thereof. The receptor, one or more domains thereof, and/or a soluble form thereof can comprise a ligand binding domain that binds to a ctDNA to be antagonized. Accordingly, the antagonist can act as a decoy that competes for binding to ctDNA, reducing binding of the ctDNA to the receptor that is associated with uptake of the ctDNA. For example, in some embodiments the antagonist is or comprises an armadillo-like protein, an APP (amyloid precursor protein) family member, a p120(ctn)/plakophilin subfamily member, an MHC protein, a GPCR, an adhesion protein, a tyrosine phosphatase, an integrin, an ion channel, a mineral transporter, a nutrient transporter, an ATPase, one or more domains thereof, or a soluble form thereof. In some embodiments the antagonist is or comprises APLP2, PKP4, HLA-A, CD41, CD97, Integrin αVβ5, PI4K2A, TMEM120A, PTK7A, Ceruloplasmin, PTPRF, SLC16A1, SLC7A5, ATP1B1, ATP2B4, PIP4K2A, P142A, one or more domains thereof, or a soluble form thereof.

An antagonist can be or can comprise a small molecule antagonist. An antagonist can be or can comprise a small chemical entity. An antagonist can be or can comprise an organic or inorganic chemical entity. In some embodiments, an antagonist has a low molecular weight (e.g., at most 1000 Daltons, at most 500 Daltons, or at most 2500 Daltons).

An antagonist can be or can comprise a polynucleotide. A polynucleotide antagonist can be, for example, DNA (such as ssDNA or dsDNA), or RNA. In some embodiments, the antagonist is or comprises a nucleic acid sequence identified in circulating tumor DNA (ctDNA), or a derivative thereof. For example, a nucleotide sequence from a ctDNA that binds to a receptor, such as a recognition sequence, can be used. In some embodiments, the nucleotide sequence from the ctDNA lacks an element that induces uptake of the ctDNA in native ctDNA. In some embodiments, the nucleotide sequence from the ctDNA is used without a cargo or associated sequence, such that substantially no cargo is delivered to the target cell upon binding of the polynucleotide to the receptor. In some embodiments, the antagonist is a polynucleotide antagonist that binds to the receptor. In some embodiments, the antagonist is a polynucleotide antagonist that binds to the ctDNA. In some embodiments, the antagonist is a polynucleotide antagonist that binds to the receptor and the ctDNA.

An antagonist can bind to a target molecule (e.g., receptor) with a K_{D} of, for example, less than about 100 mM, less than about 10 mM, less than about 1 mM, less than about 500 μM, less than about 100 μM, less than about 50 μM, less than about 10 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, less than about 900 μM, less than about 800 μM, less than about 700 μM, less than about 600 μM, less than about 500 μM, less than about 400 μM, less than about 300 μM, less than about 200 μM, less than about 100 μM, less than about 10 μM, or less than about 1 μM.

An antagonist can be contacted to a target cell or receptor to reduce uptake of ctDNA. The contacting can be in vivo. The contacting can be in vitro. The contacting can be ex vivo.

In some embodiments, the contacting comprises exposing the target cells to the antagonist at a concentration of at least 1 μM, at least 10 μM, at least 100 μM, at least 1 nM, at least 10 nM, at least 100 nM, at least 1 μM, at least 10 μM, at least 100 μM, or at least 1 mM.

In some embodiments, the contacting comprises exposing the target cells to the antagonist at a concentration of at most 1 μM, at most 10 μM, at most 100 μM, at most 1 nM, at most 10 nM, at most 100 nM, at most 1 μM, at most 10 μM, at most 100 μM, or at most 1 mM.

In some embodiments, the contacting comprises exposing the target cells to the antagonist at a concentration of about 1 μM, about 10 μM, about 100 μM, about 1 nM, about 10 nM, about 100 nM, about 1 μM, about 10 μM, about 100 μM, or about 1 mM.

In some embodiments, the contacting comprises exposing the target cells to the antagonist at a concentration of about 1 μM to about 10 μM, about 1 μM to about 100 μM, about 1 μM to about 1 nM, about 1 μM to about 10 nM, about 1 μM to about 100 nM, about 1 μM to about 1 μM, about 1 μM to about 10 μM, about 1 μM to about 100 μM, about 1 μM to about 1 mM, about 1 μM to about 10 mM, about 1 μM to about 100 mM, about 1 nM to about 10 nM, about 1 nM to about 100 nM, about 1 nM to about 1 μM, about 1 nM to about 10 μM, about 1 nM to about 100 μM, about 1 nM to about 1 mM, about 1 nM to about 10 mM, about 1 nM to about 100 mM, about 1 μM to about 10 μM, about 1 μM to about 100 μM, about 1 μM to about 1 mM, about 1 μM to about 10 mM, or about 1 μM to about 100 mM.

In some embodiments, the contacting comprises exposing the target cells to the antagonist at a concentration of at least 1 μg/mL, at least 10 μg/mL, at least 100 μg/mL, at least 1 ng/mL, at least 10 ng/mL, at least 100 ng/mL, at least 1 ag/mL, at least 10 μg/mL, at least 100 μg/mL, or at least 1 mg/mL.

In some embodiments, the contacting comprises exposing the target cells to the antagonist at a concentration of at most 1 μg/mL, at most 10 μg/mL, at most 100 μg/mL, at most 1 ng/mL, at most 10 ng/mL, at most 100 ng/mL, at most 1 μg/mL, at most 10 μg/mL, at most 100 μg/mL, or at most 1 mg/mL.

In some embodiments, the contacting comprises exposing the target cells to the antagonist at a concentration of about 1 μg/mL, about 10 μg/mL, about 100 μg/mL, about 1 ng/mL, about 10 ng/mL, about 100 ng/mL, about 1 μg/mL, about 10 μg/mL, about 100 μg/mL, or about 1 mg/mL.

In some embodiments, the contacting comprises exposing the target cells to the antagonist at a concentration of about 1 μg/mL to about 10 μg/mL, about 1 μg/mL to about 100 μg/mL, about 1 μg/mL to about 1 ng/mL, about 1 μg/mL to about 10 ng/mL, about 1 μg/mL to about 100 ng/mL, about 1 μg/mL to about 1 μg/mL, about 1 μg/mL to about 10 g/mL, about 1 μg/mL to about 100 μg/mL, about 1 μg/mL to about 1 mg/mL, about 1 μg/mL to about 10 mg/mL, about 1 μg/mL to about 100 mg/mL, about 1 ng/mL to about 10 ng/mL, about 1 ng/mL to about 100 ng/mL, about 1 ng/mL to about 1 μg/mL, about 1 ng/mL to about g/mL, about 1 ng/mL to about 100 μg/mL, about 1 ng/mL to about 1 mg/mL, about 1 ng/mL to about 10 mg/mL, about 1 ng/mL to about 100 mg/mL, about 1 μg/mL to about 10 g/mL, about 1 μg/mL to about 100 μg/mL, about 1 μg/mL to about 1 mg/mL, about 1 g/mL to about 10 mg/mL, or about 1 μg/mL to about 100 mg/mL.

In some embodiments, contacting (e.g., treating) the target cell with an antagonist (e.g., antibody) that binds the receptor (e.g., receptor protein), such as an anti-HLA-A, anti-PKP4, anti-APLP2, anti-TMEM, anti-CD41, anti-CD97, anti-Integrin αVβ5, anti-ITGB5, anti-ITGA2B, anti-Integrin 05, anti-PI4K2A, anti-PIP4K2A, anti-TMEM120A, anti-PTK7A, anti-ceruloplasmin, anti-PTPRF, anti-SLC16A1, anti-SLC7A5, anti-ATP1B1, or anti-ATP2B4 antagonist (e.g., antibody), or contacting the ctDNA with an antagonist that binds the ctDNA, reduces binding of the cargo, synthetic delivery system, polynucleotide, or ctDNA to the target cell by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99%, for example, as determined by a confocal fluorescence microscopy assay or an Amplified Luminescent Proximity Homogeneous Assay.

In some embodiments, contacting (e.g., treating) the target cell with an antagonist (e.g., antibody) that binds the receptor (e.g., receptor protein), such as an anti-HLA-A, anti-PKP4, anti-APLP2, anti-TMEM, anti-CD41, anti-CD97, anti-Integrin αVβ5, anti-ITGB5, anti-ITGA2B, anti-Integrin 05, anti-PI4K2A, anti-PIP4K2A, anti-TMEM120A, anti-PTK7A, anti-ceruloplasmin, anti-PTPRF, anti-SLC16A1, anti-SLC7A5, anti-ATP1B1, or anti-ATP2B4 antagonist (e.g., antibody), or contacting the ctDNA with an antagonist that binds the ctDNA, reduces uptake of the cargo, synthetic delivery system, ctDNA, or polynucleotide by the target cell by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99%, for example, as determined by a confocal fluorescence microscopy assay.

In some embodiments, contacting (e.g., treating) the target cell with an antagonist (e.g., antibody) that binds the receptor (e.g., receptor protein), such as an anti-HLA-A, anti-PKP4, anti-APLP2, anti-TMEM, anti-CD41, anti-CD97, anti-Integrin αVβ5, anti-Integrin 05, anti-ITGB5, anti-ITGA2B, anti-PI4K2A, anti-PIP4K2A, anti-TMEM120A, anti-PTK7A, anti-ceruloplasmin, anti-PTPRF, anti-SLC16A1, anti-SLC7A5, anti-ATP1B1, or anti-ATP2B4 antagonist (e.g., antibody), or contacting the ctDNA with an antagonist that binds the ctDNA, reduces nuclear localization of the cargo, synthetic delivery system, ctDNA, or polynucleotide in the target cell by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99%, for example, as determined by a confocal fluorescence microscopy assay.

In some embodiments, contacting (e.g., treating) the target cell with an antagonist (e.g., antibody) that binds the receptor (e.g., receptor protein), such as an anti-HLA-A, anti-PKP4, anti-APLP2, anti-TMEM, anti-CD41, anti-CD97, anti-Integrin αVβ5, anti-Integrin 05, anti-PI4K2A, anti-PIP4K2A, anti-TMEM120A, anti-PTK7A, anti-ceruloplasmin, anti-PTPRF, anti-SLC16A1, anti-SLC7A5, anti-ATP1B1, or anti-ATP2B4 antagonist (e.g., antibody), or contacting the ctDNA with an antagonist that binds the ctDNA, reduces integration of the cargo, synthetic delivery system, ctDNA, or polynucleotide into the target cell's genome by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99% for example, as determined by a metaphase chromosome spread assay.

An antagonist of a receptor that is associated with uptake of ctDNA can be administered to a subject in need thereof, for example, to reduce uptake of the ctDNA by target cells in the subject. The antagonist can be administered in a therapeutically-effective dose and via any suitable route of administration, including those disclosed herein. An average effect can be measured in a cohort of subjects.

The target cells can be cancer cells and administering the antagonist can reduce or delay development of resistance of the cancer cells to an anti-cancer therapeutic agent, such as an anti-cancer chemotherapeutic drug, kinase inhibitor, or checkpoint inhibitor. An average effect can be measured in a cohort of subjects.

The subject can have a cancer and administering the antagonist can reduce or delay development of resistance of the cancer to an anti-cancer therapeutic agent, such as an anti-cancer chemotherapeutic drug, kinase inhibitor, or checkpoint inhibitor. An average effect can be measured in a cohort of subjects.

The subject can have a cancer and administering the antagonist can reduce or delay metastasis of the cancer, for example, prolong the average time to metastasis, reduce the average size of metastases, or reduce the incidence of metastasis in a cohort of subjects.

The subject can have a cancer and administering the antagonist can reduce or delay tumor growth or progression of the cancer to a more advanced stage, for example, using suitable criteria for cancer progression and/or tumor growth (e.g., International Uniform Response Criteria for Multiple Myeloma, or RECIST criteria). An average effect can be measured in a cohort of subjects.

The antagonist can be administered by any suitable route, for example, systemically, locally, parenterally, intravenously, subcutaneously, intramuscularly, intratumorally, by inhalation, dermally, topically, orally, sublingually, intrathecally, transdermally, intranasally, via a peritoneal route, directly into the brain (e.g., via and intracerebral ventricle route).

In some embodiments, contacting (e.g., treating) a ctDNA, synthetic delivery system, or polynucleotide with an antagonist (e.g., antibody) can reduce binding, uptake, or nuclear localization of a ctDNA, synthetic delivery system, cargo, or polynucleotide by the target cell, for example, as determined by a confocal fluorescence microscopy assay.

In some embodiments, contacting (e.g., treating) a ctDNA, synthetic delivery system, or polynucleotide with an antagonist (e.g., anti-DNA antibody, such as an anti-dsDNA antibody) reduces binding of the ctDNA, cargo, synthetic delivery system, or polynucleotide to the receptor or target cell by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99%, for example, as determined by a confocal fluorescence microscopy assay or an Amplified Luminescent Proximity Homogeneous Assay.

In some embodiments, contacting (e.g., treating) a ctDNA, synthetic delivery system, or polynucleotide with an antagonist (e.g., anti-DNA antibody, such as an anti-dsDNA antibody) reduces uptake of the ctDNA, cargo, synthetic delivery system, or polynucleotide by the target cell by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99%, for example, as determined by a confocal fluorescence microscopy assay.

In some embodiments, contacting (e.g., treating) a ctDNA, synthetic delivery system, or polynucleotide with an antagonist (e.g., anti-DNA antibody, such as an anti-dsDNA antibody) reduces nuclear localization of the ctDNA, cargo, synthetic delivery system, or polynucleotide in the target cell by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99%, for example, as determined by a confocal fluorescence microscopy assay.

In some embodiments, contacting (e.g., treating) a ctDNA, synthetic delivery system, or polynucleotide with an antagonist (e.g., anti-DNA antibody, such as an anti-dsDNA antibody) reduces integration of the ctDNA, cargo, synthetic delivery system, or polynucleotide into the target cell's genome by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99% for example, as determined by a metaphase chromosome spread assay.

In some embodiments, contacting (e.g., treating) a ctDNA, synthetic delivery system, polynucleotide, target cell, or receptor with an antagonist disclosed herein reduces colocalization of the ctDNA, synthetic delivery system, or polynucleotide with the receptor by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99%, for example, as determined by a confocal fluorescence microscopy assay.

Determining Expression of a Receptor Associated with Uptake of ctDNA

Methods disclosed herein can include measuring an expression level of a receptor that is associated with uptake of a ctDNA, polynucleotide, transposable element, or synthetic delivery system. For example, a biological sample can be assayed to determine whether the receptor is expressed in the biological sample at a level that is associated with uptake of the ctDNA, polynucleotide, transposable element, or synthetic delivery system.

An assay can be performed on a biological sample to measure an expression level of a receptor that is associated with uptake of a ctDNA, polynucleotide, transposable element, or synthetic delivery system. The assay can measure expression of the receptor at protein level and/or mRNA level, e.g., can quantify or qualify an expression level of the receptor protein, or an mRNA encoding the receptor. Any suitable assay can be used to determine the expression level of the receptor. Non-limiting examples of assays that can be used include immunohistochemistry, ELISA, multiplex immunoassay, mass spectrometry (e.g., targeted or untargeted), fluorescence microscopy, flow cytometry, quantitative PCR (e.g., quantitative reverse transcriptase real time PCR), digital PCR, and RNA sequencing. In some embodiments, the assay is quantitative. In some embodiments, the assay is semi-quantitative, for example, provides a relative expression level to a control.

An assay can utilize an agent that binds to the receptor that is associated with uptake of the ctDNA, polynucleotide, transposable element, or synthetic delivery system. The agent that binds to the receptor can be, for example, an antibody or antigen-binding fragment thereof. The agent that binds to the receptor can be or comprise a polypeptide. The agent that binds to the receptor can be or comprise a ligand of the receptor. The agent can be conjugated directly or indirectly to a detectable probe, for example, a fluorescent molecule. The agent can be conjugated directly or indirectly to a factor that provides a signal relative to the amount of receptor detected, for example, an enzyme that mediates a proportional fluorescent, luminescent, or colorimetric change in response to a substrate (e.g., luciferase/luciferin, horse radish peroxidase/3,3′,5,5′-Tetramethylbenzidine (TMB), or alkaline phosphatase).

In some embodiments, a method of determining an expression level of a receptor comprises contacting the receptor or biological sample with an antibody that binds to the receptor, and contacting the biological sample or antibody with a detection agent that induces a detectable signal based on a presence of or proportional to a level of the antibody bound to the receptor.

In some embodiments, the disclosure provides a method of determining an expression level of a receptor that comprises an immunohistochemistry assay. The biological sample can be appropriately prepared for immunohistochemistry (for example, collected, fixed, embedded, sectioned, mounted, de-paraffinized, and subject to antigen retrieval). The method can comprise blocking endogenous target activity, blocking nonspecific binding sites, sample labelling (e.g., immunodetection, counterstaining, sealing), visualization/imaging, or a combination thereof.

An assay can utilize an agent that binds to an mRNA that encodes the receptor, for example, a nucleic acid that is complementary or reverse complementary to a portion of the mRNA. The agent can be conjugated directly or indirectly to a detectable probe, for example, a fluorescent molecule.

The method or assay can comprise determining that the receptor is expressed at a level that is associated with the uptake of the ctDNA, polynucleotide, transposable element, or synthetic delivery system. A level that is associated with the uptake of the ctDNA, polynucleotide, transposable element, or synthetic delivery system can be determined, for example, by comparing levels of expression of the receptor (e.g., via an assay disclosed herein) among samples of target cells that exhibit various levels of uptake of the ctDNA, polynucleotide, transposable element, or synthetic delivery system (e.g., detectably labelled, such as fluorescently labelled). The samples of target cells can be primary cells or cell lines. The samples of target cells can comprise artificially modified levels of expression of the receptor, for example, via knockdown or knockout of expression of the receptor, and/or via inducing expression from artificial constructs using promoters that drive different levels of expression, or can be induced to drive different levels of expression via an inducible and/or repressible mechanism. In some embodiments, a level of receptor expression associated with uptake of the ctDNA, polynucleotide, transposable element, or synthetic delivery system can vary by receptor, ctDNA, polynucleotide, synthetic delivery system, transposable element, synthetic construct, and/or target cell type.

A biological sample can comprise or can be a liquid. A biological sample can be a liquid biopsy. A biological sample can be or can comprise, for example, blood (e.g., whole blood).

A biological sample can be or can comprise a solid. A biological sample can be or can comprise a solid tissue sample from any organ or tissue. A biological sample can be or can comprise a biopsy that comprises tumor tissue or is suspected to comprise tumor tissue. A biological sample can comprise tumor tissue, for example, of any cancer or tumor type disclosed herein. A biological sample can comprise cancer cells, for example, of any cancer or tumor type disclosed herein. A biological sample can comprise predominantly cells from a specific organ or from a tissue within a specific organ. An organ can refer to a group of cells, for example, in a liquid or solid form, with or without an extracellular matrix. In some embodiments, cells within an organ (e.g., in a healthy subject) have a biological function that distinguishes them from other cells outside the organ. A biological sample can comprise or can be a tissue sample. A biological sample can be obtained as part of a biopsy. A biological sample can be obtained as part of a surgery.

A biological sample can comprise biological material that is fresh frozen (FF), fixed (e.g., in neutral buffered formalin or any other tissue fixative), formalin fixed paraffin embedded (FFPE), cryopreserved, incubated in RNA and/or protein stabilizing reagents, or otherwise preserved or stabilized for the maximum recovery of RNA and/or protein from within the sample.

In some embodiments, a biological sample is isolated from a subject that is being screened for cancer, is suspected of having cancer, is diagnosed with cancer, or is being monitored for cancer recurrence or relapse. The biological sample can comprise primary tumor tissue, metastatic tumor tissue, precancerous tissue, and/or tissue that is believed to contain tumor cells or precancerous cellular changes. The biological sample can contain tumor-infiltrating immune cells or other cells in the tumor tissue or in adjacent normal tissue. The biological sample can be a biological sample encountered in clinical pathology, including but not limited to, sections of tissues such as biopsy or tissue removed during surgical or other procedures, bodily fluids, autopsy samples, or frozen sections taken for histological or histopathological purposes. Such biological samples can include blood and blood fractions or products, sputum, effusion, cheek cells tissue, patient-derived cultured cells (e.g., primary cultures, explants, and transformed cells), stool, urine, other biological or bodily fluids. etc.

A biological sample can be obtained from a subject before a treatment (e.g., administration of a therapeutic agent or synthetic delivery system), during a treatment, or after a treatment. In some embodiments, biological samples are obtained from a subject before a treatment, during the treatment, and/or after the treatment. In some embodiments, an initial biological sample is obtained from a subject and a subsequent biological sample is obtained from the subject later (e.g., months or years later, for comparison).

In some embodiments, a biological sample is or has been examined by a certified clinical pathologist. In some embodiments, the biological sample is subjected to laboratory diagnostic tests (such as immunohistochemical assays or array CGH) to confirm that the biological sample is diseased or non-diseased and is of the assumed sample type (e.g., the tissue, biological fluid, cell type, cell line, cancer type etc.).

A biological sample can be a control biological sample obtained from a control subject. The control subject can be, for example, a normal subject that does not have a given cancer. A control biological sample can be or can comprise a sample that is healthy or normal. A control biological sample can be from a second/different type of cancer (for example, pancreatic cancer can be a control for multiple myeloma, and vice versa). A control biological sample can be or can comprise a sample from a tissue that is healthy or normal. A tissue that is healthy or normal can lack a specific pathological diagnosis (e.g., disease diagnosis). For example, the tissue that is healthy or normal can lack a cancer diagnosis. A control biological sample can be used for comparison, for example, in a process of determining a level of expression of a receptor that is associated with uptake of a ctDNA, polynucleotide, transposable element, or synthetic delivery system. The control biological sample can be used for determining whether uptake is specific for a given cancer type or cell type, for example.

In some embodiments, receptors or nucleic acid constructs of the present disclosure can be used for diagnostic and monitoring purposes in various chronic, infectious or inherited (e.g., genetic) diseases, including cancer and certain disorders related to, for example, blood cells (e.g., anemia, thalassemia, hemophilia, or platelet disorders). In some cases, the presence or expression level of a receptor and/or nucleic acid sequence as disclosed herein can be used as a biomarker for a particular disease or condition, or can be used to monitor response to a particular therapeutic intervention (e.g., chemotherapy, targeted therapy, immunotherapy, or cell and gene therapy). In other instances, a receptor, synthetic delivery system, or polynucleotide disclosed herein can be used as a diagnostic. For example, the integration of a polynucleotide or a part thereof into a genome of a cell can be used as a measurement or marker to determine the degree of integration of a cargo nucleic acid sequence (e.g., a therapeutic gene sequence) into a genome. In other cases, cell targeting and genomic integration of a polynucleotide (or delivery system) described herein can be used as a marker for a particular biological effect. In an example, the degree of uptake or genomic integration of a cargo, polynucleotide, or part thereof can be a marker or measurement for a therapeutic effect, e.g., cell killing.

### Therapeutic Agent Administration

Methods disclosed herein can comprise administering a therapeutic agent to a subject. The subject can be a subject in need thereof, for example, that has a condition to be treated. A therapeutic agent administered to the subject can be or can comprise a synthetic delivery system disclosed herein, e.g., for delivery of a cargo. A therapeutic agent administered to the subject can be or can comprise an antagonist, e.g., of a receptor associated with uptake of ctDNA.

The therapeutic agent (e.g., synthetic delivery system or antagonist) can be administered to a subject based on a level of expression of a receptor associated with uptake of ctDNA. For example, if the subject expresses the receptor associated with uptake of ctDNA (e.g., at a detectable level, or at least at a level associated with uptake of ctDNA), the therapeutic agent can be administered to the subject. Accordingly, an assay can be performed on a biological sample from the subject to determine whether the biological sample exhibits expression of the receptor (e.g., at a detectable level, or at least at a level associated with uptake of ctDNA). The assay can be an assay disclosed herein for determining the expression level of the receptor, for example, an immunohistochemistry, ELISA, multiplex immunoassay, mass spectrometry (e.g., targeted or untargeted), flow cytometry, fluorescence microscopy, quantitative PCR (e.g., quantitative reverse transcriptase real time PCR), digital PCR, or RNA sequencing assay on a biological sample.

In some embodiments, a subject is selected for administration of the therapeutic agent if the biological sample exhibits expression of the receptor at a detectable level. In some embodiments, a subject is selected for administration of the therapeutic agent if the biological sample exhibits expression of the receptor at least at a level associated with uptake of ctDNA. In some embodiments, a subject is not selected for administration of the therapeutic agent if the biological sample does not exhibit expression of the receptor at a detectable level. In some embodiments, a subject is not selected for administration of the therapeutic agent if the biological sample exhibits expression of the receptor below a level associated with uptake of ctDNA. The level of expression of the receptor can be used in conjunction with other data or criteria for determining whether to administer the therapeutic agent to the subject, for example, typing, staging, mutational burden, receptor expression, drug resistance/sensitivity, etc. of the cancer.

In some embodiments, methods of the disclosure comprise identifying a suitable therapeutic agent that can benefit a subject in need thereof. In some embodiments, methods of the disclosure comprise identifying a therapeutic agent that is unlikely to benefit a subject in need thereof.

Non-limiting examples of therapeutic agents that can be administered to a subject include vaccines (e.g., mRNA vaccines), AKT inhibitors, alkylating agents, anti-angiogenic agents, antibiotic agents, antifolates, anti-hormone therapies, anti-inflammatory agents, antimetabolites, anti-VEGF agents, apoptosis promoting agents, aromatase inhibitors, ATM regulators, biologic agents, BRAF inhibitors, BTK inhibitors, CAR-T cells, CAR-NK cells, CDK inhibitors, cell growth arrest inducing-agents, cell therapies, chemotherapy, cytokine therapies, cytotoxic drugs, demethylating agents, differentiation-inducing agents, estrogen receptor antagonists, gene therapy agents, growth factor inhibitors, growth factor receptor inhibitors, HDAC inhibitors, heat shock protein inhibitors, hematopoietic stem cell transplantation (HSCT), hormones, hydrazine, immune checkpoint inhibitors, immumomodulators, immunosuppressants, kinase inhibitors, KRAS inhibitors, matrix metalloproteinase inhibitors, MEK inhibitors, mitotic inhibitors, mTOR inhibitors, multi-specific (e.g., bispecific) immune cell engagers, multi-specific (e.g., bispecific) killer cell engagers, multi-specific (e.g., bispecific) T cell engagers, nitrogen mustards, oncolytic viruses, oxazaphosphorines, p53 reactivating agents, plant alkaloids, platinum-based agents, proteasome inhibitors, purine analogs, purine antagonists, pyrimidine antagonists, radiation therapy, ribonucleotide reductase inhibitors, signal transduction inhibitors, RNA silencing (e.g., RNAi) agents, gene editing agents, a CRISPR/Cas systems or a component thereof, an RNA replacement therapy, a protein replacement therapy, a gene therapy, antibody drug conjugates, surgery, taxanes, therapeutic antibodies, topoisomerase inhibitors, transgenic T cells, tyrosine kinase inhibitors, and vinca alkaloids.

A therapeutic agent can be, for example, an anti-cancer therapeutic agent. Non-limiting examples of anti-cancer therapeutic agents include cancer vaccines (e.g., mRNA vaccines), AKT inhibitors, alkylating agents, anti-angiogenic agents, antibiotic agents, antifolates, anti-hormone therapies, anti-inflammatory agents, antimetabolites, anti-VEGF agents, apoptosis promoting agents, aromatase inhibitors, ATM regulators, biologic agents, BRAF inhibitors, BTK inhibitors, CAR-T cells, CAR-NK cells, CDK inhibitors, cell growth arrest inducing-agents, cell therapies, chemotherapy, cytokine therapies, cytotoxic drugs, demethylating agents, differentiation-inducing agents, estrogen receptor antagonists, gene therapy agents, growth factor inhibitors, growth factor receptor inhibitors, HDAC inhibitors, heat shock protein inhibitors, hematopoietic stem cell transplantation (HSCT), hormones, hydrazine, immune checkpoint inhibitors, immumomodulators, kinase inhibitor, KRAS inhibitors, matrix metalloproteinase inhibitors, MEK inhibitors, mitotic inhibitors, mTOR inhibitors, multi-specific (e.g., bispecific) immune cell engagers, multi-specific (e.g., bispecific) killer cell engagers, multi-specific (e.g., bispecific) T cell engagers, nitrogen mustards, oncolytic viruses, oxazaphosphorines, p53 reactivating agents, plant alkaloids, platinum-based agents, proteasome inhibitors, purine analogs, purine antagonists, pyrimidine antagonists, radiation therapy, ribonucleotide reductase inhibitors, signal transduction inhibitors, RNA silencing (e.g., RNAi) agents, gene editing agents, a CRISPR/Cas systems or a component thereof, an RNA replacement therapy, a protein replacement therapy, a gene therapy, antibody drug conjugates, surgery, taxanes, therapeutic antibodies, topoisomerase inhibitors, transgenic T cells, tyrosine kinase inhibitors, and vinca alkaloids.

A therapeutic agent can be a drug. A therapeutic agent can be a non-cancer therapeutic, for example, a therapeutic for a metabolic disease, autoimmune disease, neurological disease, or degenerative disease. A therapeutic agent can be, for example, a vaccine (e.g., cancer vaccine), a drug, an immunotherapy, an immune checkpoint inhibitor, a kinase inhibitor, a small molecule, a chemotherapeutic agent, a radiotherapy, a biologic, or any combination thereof.

A therapeutic agent can modulate (e.g., increase or decrease) activity of a target gene (e.g., an aberrantly expressed gene), or a product encoded by the target gene, such as a protein or RNA. A therapeutic agent can modulate (e.g., increase or decrease) expression of a target gene (e.g., an aberrantly expressed gene). A therapeutic agent can modulate (e.g., increase or decrease) activity of a ligand or receptor of a target gene (e.g., an aberrantly expressed gene). In some embodiments, a therapeutic agent can alter the gene product of an aberrantly-expressed gene, e.g., by targeting the gene product, the transcript of the gene, or epigenetic factors that influence a property of the gene (e.g., expression). Non-limiting examples include targeting the protein that the gene encodes, reducing expression levels of the gene using gene therapy or RNAi, and using RNA vaccines to establish an immune response.

Methods of the disclosure can be used to identify a therapeutic agent that can be used in the treatment of a disease or condition, such as a cancer. Methods of the disclosure can be used to treat a condition in a subject in need thereof, such as a cancer. The cancer can be a solid tumor or a liquid cancer, e.g., leukemia or lymphoma. In some embodiments, the cancer is a solid tumor. In some embodiments, the cancer is a hematological cancer, for example, a B cell cancer, such as multiple myeloma. In some embodiments, the cancer is a pancreatic cancer. In some embodiments, the cancer is colon cancer or colorectal cancer. In some embodiments, the cancer is a lung cancer.

In some embodiments, the cancer comprises bladder cancer, brain cancer (e.g., astrocytoma, glioblastoma, meningioma, or oligodendroglioma), breast cancer (e.g., ER+, PR+, HER2+, or triple-negative breast cancer), bone cancer, cervical cancer, colon cancer, colorectal cancer, esophageal cancer, head and neck cancer, kidney cancer, liver cancer, lung cancer, medullary thyroid cancer, mouth cancer, nose cancer, ovarian cancer (e.g., mucinous, endometrioid, clear cell, or undifferentiated), pancreatic cancer, renal cancer, skin cancer, stomach cancer, throat cancer, thyroid cancer, or uterus cancer. In some embodiments, the cancer comprises bladder cancer, brain cancer, breast cancer, colon cancer, colorectal cancer, lung cancer, or ovarian cancer. In some embodiments, the cancer is lung cancer. In some embodiments, the cancer is brain cancer. In some embodiments, the cancer is breast cancer, e.g., triple-negative breast cancer. In some embodiments, the cancer is ovarian cancer. In some embodiments, the cancer is bladder cancer.

In some embodiments, the cancer is a carcinoma. In some embodiments, the cancer is a sarcoma. In some embodiments, the cancer is an adenoma. In some embodiments, the cancer is an adenocarcinoma.

In some embodiments, the cancer is of unknown primary tissue. In some embodiments, a method disclosed herein is used to identify the primary tissue type. In some embodiments, the cancer is a neuroendocrine tumor.

A subject (e.g., treated using a method disclosed herein, and/or the source of a biological sample) can be a mammal. In some embodiments the subject is a human. In some embodiments, the subject is a mouse, a rat, a cat, a dog, a rabbit, a cow, a horse, a goat, a monkey, a cynomolgus monkey, or a lamb. In some embodiments, the subject is a primate. In some embodiments, the subject is a non-human primate. In some embodiments, the subject is a non-rodent subject. A subject can be a female subject. A subject can be a male subject.

In practicing the methods of treatment or use provided herein, therapeutically-effective amounts of the therapeutic agents described herein can be administered in pharmaceutical compositions to a subject having a disease or condition to be treated. A therapeutically-effective amount can vary widely depending on the severity of the disease, the age and relative health of the subject, the potency of the compounds used, and other factors.

Disclosed herein, in some embodiments, is a method of delivering a cargo to a target cell. The method can comprise contacting a target cell or a receptor on the target cell with a synthetic delivery system disclosed herein that comprises a polynucleotide.

In some embodiments, a method of delivering a cargo disclosed herein utilizing a synthetic delivery system can be used to treat cancer, an inflammatory disease, or an autoimmune disease. In some embodiments, a method of delivering a cargo disclosed herein utilizing a synthetic delivery system can be used to administer a vaccine.

A treatment can comprise administering to a subject a synthetic delivery system or antagonist. A synthetic delivery system or antagonist disclosed herein can be administered to a subject intravenously, subcutaneously, intramuscularly, intranasally, by inhalation, intrademally, dermally, topically, orally, sublingually, intrathecally, transdermally, intranasally, intracerebrally, intraspinally, intraarticularly, ophthalmically, rectally, via a peritoneal route, or directly into the brain, e.g., via an intracerebral ventricle route. A synthetic delivery system or antagonist can be administered via absorption through epithelial or mucocutaneous linings (e.g., oral mucosa, rectal and intestinal mucosa).

A synthetic delivery system, antagonist, or therapeutic agent described herein can be administered before, during, or after the occurrence of a disease or condition, and the timing of administering a composition containing the synthetic delivery system, antagonist, or therapeutic agent can vary. For example, the composition can be used as a prophylactic and can be administered continuously to subjects with a propensity to conditions or diseases in order to lessen a likelihood of the occurrence of the disease or condition. The composition can be administered to a subject already suffering from a disease or condition, in an amount sufficient to cure or at least partially arrest the symptoms of the disease or condition, or to cure, heal, improve, or ameliorate the condition. The composition can be administered to a subject during or as soon as practical after the onset of the symptoms.

Multiple synthetic delivery systems, antagonists, or therapeutic agents disclosed herein can be administered in any order or simultaneously. If simultaneously, the multiple synthetic delivery systems, antagonists, or therapeutic agents can be provided in a single, unified form, or in multiple forms, for example, as multiple separate injections or infusions. The synthetic delivery systems, antagonists, or therapeutic agents can be packed together or separately, in a single package or in a plurality of packages. One or all of the synthetic delivery systems, antagonists, or therapeutic agents can be given in multiple doses.

The methods and compositions of the present disclosure may be useful for altering the phenotype of a target cell and/or a target cell population. In some cases, phenotypic alteration of target cells in a subject can improve the therapeutic and/or clinical response of the subject to a therapeutic intervention. In some embodiments, methods and compositions of the disclosure can be used to alter sensitivity of a target cell or target cell population to a drug, for example, increase sensitivity of cancer cells to an anti-cancer drug, or limiting or reducing a decrease in sensitivity of cancer cells to the anti-cancer drug.

A composition or method of the present disclosure can be used to elicit an immune response in a subject, e.g., upon administration of a synthetic delivery system. For example, an immune response can be elicited by administering nucleic acid constructs that comprise a cargo nucleic acid sequence coding for one or more antigenic or immunogenic peptides or proteins, and such immunogenic peptides or proteins can elicit an immune response in the subject upon expression. A synthetic delivery system elicits an immune response based on the presence of cytosolic nucleic acid (e.g., DNA). In some embodiments, a synthetic delivery system elicits an immune response based on expression of a transgene from a nucleic acid cargo, for example, encoding a cytokine.

In some embodiments, the high specificity and/or efficiency by which a synthetic delivery system disclosed herein delivers to targets (e.g., multiple myeloma cells) cells while largely sparing non-target cells (e.g., other bone marrow cells) supports use of synthetic delivery systems disclosed herein as therapeutic vectors.

In some embodiments, a synthetic delivery system is used to visualize and/or track a disease or condition (e.g., cancer) in vivo, e.g., by delivering a chemical dye (e.g., a fluorescent dye) or a radioactive isotope to one or more cells associated with the disease or conditions. In another example, tumor cells can be visualized and tracked in vivo by delivering, for example, a chemical dye (e.g., a fluorescent dye), a radioactive isotope, or contrast agents to the tumor site(s) (e.g., primary tumor site and metastatic sites) by using a synthetic delivery system disclosed herein.

A synthetic delivery system, polynucleotide, or pharmaceutical composition can be administered in combination with one or more other therapeutic agents, for example, small molecule drugs, immunotherapeutic agents (e.g., immune check point blocker), therapeutic antibodies, or any other therapeutic agent disclosed herein.

Identification of Receptors for ctDNA

Various assays can be used to demonstrate a role of a receptor in cellular binding, uptake, nuclear localization, and/or genomic integration of a ctDNA, synthetic delivery system, polynucleotide, or cargo.

The disclosure provides methods of identifying a receptor or fragment thereof that binds to and/or mediates uptake of a ctDNA, synthetic delivery system, polynucleotide, or cargo by a target cell. The method can comprise contacting cell lysate to the ctDNA, a synthetic delivery system, or polynucleotide that comprises ctDNA or a derivative thereof (e.g., recognition sequence). The cell lysate can be generated from a population of target cells. The ctDNA or synthetic delivery system that is bound to a receptor can be isolated, for example, by a pulldown assay. The receptor or fragment thereof that is bound to the ctDNA or synthetic delivery system can be identified, for example, by mass spectrometry. The ctDNA or synthetic delivery system can be labelled or tagged to facilitate isolation (e.g., pulldown) of the bound receptor or fragment thereof. For example, the ctDNA or synthetic delivery system can be biotinylated, or can comprise a tag sequence that is bound by a reagent, such as an antibody.

Knockout, knockdown, or disruption of a gene that encodes a receptor or a component thereof can be used to demonstrate a role of the receptor in cellular binding, uptake, nuclear localization, and/or genomic integration of a cargo, synthetic delivery system, ctDNA, or polynucleotide. Knockout can comprise removing all or part of the gene that encodes the receptor, for example, using a nuclease system with or without a repair template (e.g., CRISPR/Cas mediated cleavage with provision of a template for homology-directed repair). Knockout can comprise disruption of the gene, for example, using a transposon/transposase system or a CRISPR/Cas system. Knockdown can comprise targeting an RNA transcript of a gene encoding the receptor or component thereof for degradation, for example, using an siRNA or a targeted endonuclease system that degrades the RNA transcript.

In some embodiments, knockout, knockdown, or disruption of a gene that encodes a receptor or a component thereof reduces binding of the cargo, synthetic delivery system, ctDNA, or polynucleotide to the target cell by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99%, for example, as determined by a confocal fluorescence microscopy assay or an ALPHA assay.

In some embodiments, knockout, knockdown, or disruption of a gene that encodes a receptor or a component thereof reduces uptake of the cargo, synthetic delivery system, ctDNA, or polynucleotide by the target cell by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99%, for example, as determined by a confocal fluorescence microscopy assay.

In some embodiments, knockout, knockdown, or disruption of a gene that encodes a receptor or a component thereof reduces nuclear localization of the cargo, synthetic delivery system, ctDNA, or polynucleotide by the target cell by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99%, for example, as determined by a confocal fluorescence microscopy assay.

In some embodiments, knockout, knockdown, or disruption of a gene that encodes a receptor or a component thereof reduces integration of the cargo, synthetic delivery system, ctDNA, or polynucleotide into the target cell's genome by at least about 5%, at least about 10%, at least about 15%, at least about 20%, at least about 30%, at least about 40%, at least about 50%, at least about 60%, at least about 70%, at least about 80%, at least about 90%, at least about 95%, or at least about 99% for example, as determined by a metaphase chromosome spread assay or sequencing.

The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length, maximizing the number of matches and minimizes the number of gaps. Default settings can be used.

### Embodiments

Embodiment 1. A method of reducing uptake of circulating tumor-derived DNA (ctDNA) by target cells in a subject, the method comprising administering to the subject an antagonist of a receptor that is associated with cellular uptake of the ctDNA, thereby reducing the uptake of the ctDNA by the target cells in the subject.

Embodiment 2. The method of embodiment 1, wherein the antagonist comprises an antibody or an antigen-binding fragment thereof.

Embodiment 3. The method of embodiment 1 or embodiment 2, wherein the antagonist comprises an IgG antibody.

Embodiment 4. The method of embodiment 1 or embodiment 2, wherein the antagonist comprises a polypeptide.

Embodiment 5. The method of embodiment 1, wherein the antagonist comprises a ligand of the receptor.

Embodiment 6. The method of embodiment 1, wherein the antagonist comprises a small molecule antagonist of the receptor.

Embodiment 7. The method of embodiment 1, wherein the antagonist comprises a polynucleotide.

Embodiment 8. The method of embodiment 1, wherein the antagonist comprises a polynucleotide, wherein the polynucleotide comprises a transposable element.

Embodiment 9. The method of embodiment 1, 7, or 8, wherein the antagonist comprises DNA.

Embodiment 10. The method of any one of embodiments 1 and 7-9, wherein the antagonist comprises double stranded DNA.

Embodiment 11. The method of any one of embodiments 1 and 7-10, wherein the antagonist comprises a nucleic acid sequence identified in circulating tumor DNA (ctDNA), or a derivative thereof.

Embodiment 12. The method of any one of embodiments 1-11, wherein the receptor comprises an armadillo-like protein.

Embodiment 13. The method of any one of embodiments 1-12, wherein the receptor comprises a p120(ctn)/plakophilin subfamily member.

Embodiment 14. The method of any one of embodiments 1-11, wherein the receptor comprises an APP (amyloid precursor protein) family member.

Embodiment 15. The method of any one of embodiments 1-11, wherein the receptor comprises an MHC protein.

Embodiment 16. The method of any one of embodiments 1-11, wherein the receptor comprises a GPCR.

Embodiment 17. The method of any one of embodiments 1-11, wherein the receptor comprises an adhesion protein.

Embodiment 18. The method of any one of embodiments 1-11, wherein the receptor comprises a tyrosine phosphatase.

Embodiment 19. The method of any one of embodiments 1-11, wherein the receptor comprises an integrin.

Embodiment 20. The method of any one of embodiments 1-11, wherein the receptor comprises an ion channel.

Embodiment 21. The method of any one of embodiments 1-11, wherein the receptor comprises a mineral transporter.

Embodiment 22. The method of any one of embodiments 1-11, wherein the receptor comprises a nutrient transporter.

Embodiment 23. The method of any one of embodiments 1-11, wherein the receptor comprises an ATPase.

Embodiment 24. The method of any one of embodiments 1-11, wherein the receptor comprises APLP2.

Embodiment 25. The method of any one of embodiments 1-11, wherein the receptor comprises PKP4.

Embodiment 26. The method of any one of embodiments 1-11, wherein the receptor comprises HLA-A.

Embodiment 27. The method of any one of embodiments 1-11, wherein the receptor comprises CD41.

Embodiment 28. The method of any one of embodiments 1-11, wherein the receptor comprises CD97.

Embodiment 29. The method of any one of embodiments 1-11, wherein the receptor comprises Integrin αVβ5.

Embodiment 30. The method of any one of embodiments 1-11, wherein the receptor comprises PI4K2A.

Embodiment 31. The method of any one of embodiments 1-11, wherein the receptor comprises TMEM120A.

Embodiment 32. The method of any one of embodiments 1-11, wherein the receptor comprises PTK7A.

Embodiment 33. The method of any one of embodiments 1-11, wherein the receptor comprises Ceruloplasmin.

Embodiment 34. The method of any one of embodiments 1-11, wherein the receptor comprises PTPRF.

Embodiment 35. The method of any one of embodiments 1-11, wherein the receptor comprises SLC16A1.

Embodiment 36. The method of any one of embodiments 1-11, wherein the receptor comprises SLC7A5.

Embodiment 37. The method of any one of embodiments 1-11, wherein the receptor comprises ATP1BL.

Embodiment 38. The method of any one of embodiments 1-11, wherein the receptor comprises ATP2B4.

Embodiment 39. The method of any one of embodiments 1-11, wherein the receptor comprises P142A.

Embodiment 40. The method of any one of embodiments 1-39, wherein the target cells comprise cancer cells.

Embodiment 41. The method of any one of embodiments 1-40, wherein the target cells comprise leukocytes.

Embodiment 42. The method of any one of embodiments 1-41, wherein the target cells comprise lymphocytes.

Embodiment 43. The method of any one of embodiments 1-42, wherein the target cells comprise B lymphocytes.

Embodiment 44. The method of any one of embodiments 1-43, wherein the target cells comprise plasma cells.

Embodiment 45. The method of any one of embodiments 1-44, wherein the target cells comprise multiple myeloma cells.

Embodiment 46. The method of any one of embodiments 1-40, wherein the target cells comprise pancreatic cells.

Embodiment 47. The method of any one of embodiments 1-40, wherein the target cells comprise pancreatic cancer cells.

Embodiment 48. The method of any one of embodiments 1-40, wherein the target cells comprise gastrointestinal cells.

Embodiment 49. The method of any one of embodiments 1-40, wherein the target cells comprise colorectal cancer cells.

Embodiment 50. The method of any one of embodiments 1-49, wherein the target cells are cancer cells and method reduces development of resistance of the cancer cells to a therapeutic agent.

Embodiment 51. The method of any one of embodiments 1-50, wherein the subject has a cancer and the method reduces development of resistance of the cancer to a therapeutic agent.

Embodiment 52. The method of any one of embodiments 1-51, wherein the subject has a cancer and the method reduces metastasis of the cancer.

Embodiment 53. The method of any one of embodiments 1-52, wherein the subject has a cancer and the method reduces progression of the cancer.

Embodiment 54. The method of any one of embodiments 1-49, wherein the target cells are cancer cells and method delays development of resistance of the cancer cells to a therapeutic agent.

Embodiment 55. The method of any one of embodiments 1-49, wherein the subject has a cancer and the method delays development of resistance of the cancer to a therapeutic agent.

Embodiment 56. The method of any one of embodiments 1-49, wherein the subject has a cancer and the method delays metastasis of the cancer.

Embodiment 57. The method of any one of embodiments 1-49, wherein the subject has a cancer and the method delays progression of the cancer.

Embodiment 58. A method of reducing uptake of circulating tumor-derived DNA (ctDNA) by target cells, the method comprising contacting the target cells with an antagonist of a receptor that is associated with uptake of the ctDNA, thereby reducing the uptake of the ctDNA by the target cells.

Embodiment 59. The method of embodiment 58, wherein the uptake of the ctDNA by the target cells is reduced by at least 5% as determined by a confocal fluorescence microscopy assay.

Embodiment 60. The method of embodiment 58 or embodiment 59, wherein the target cells are exposed to the antagonist at a concentration of at least 1 ng/mL.

Embodiment 61. The method of any one of embodiments 58-60, wherein the target cells are exposed to the antagonist at a concentration of about 1 g/mL.

Embodiment 62. The method of any one of embodiments 58-61, wherein the contacting is in vivo.

Embodiment 63. The method of any one of embodiments 58-61, wherein the contacting is in vitro or ex vivo.

Embodiment 64. The method of any one of embodiments 58-63, wherein the antagonist comprises an antibody or an antigen-binding fragment thereof.

Embodiment 65. The method of any one of embodiments 58-64, wherein the antagonist comprises an IgG antibody.

Embodiment 66. The method of any one of embodiments 58-63, wherein the antagonist comprises a polypeptide.

Embodiment 67. The method of any one of embodiments 58-63 and 66, wherein the antagonist comprises a ligand of the receptor.

Embodiment 68. The method of any one of embodiments 58-63, wherein the antagonist comprises a small molecule antagonist of the receptor.

Embodiment 69. The method of any one of embodiments 58-63, wherein the antagonist comprises a polynucleotide.

Embodiment 70. The method of any one of embodiments 58-63, wherein the antagonist comprises DNA.

Embodiment 71. The method of any one of embodiments 58-63, wherein the antagonist comprises double stranded DNA.

Embodiment 72. The method of any one of embodiments 58-63, wherein the antagonist comprises a nucleic acid sequence identified in circulating tumor DNA (ctDNA), or a derivative thereof.

Embodiment 73. The method of any one of embodiments 58-63 and 72, wherein the antagonist comprises a polynucleotide, wherein the polynucleotide comprises a transposable element.

Embodiment 74. The method of any one of embodiments 58-73, wherein the receptor comprises an armadillo-like protein.

Embodiment 75. The method of any one of embodiments 58-74, wherein the receptor comprises a p120(ctn)/plakophilin subfamily member.

Embodiment 76. The method of any one of embodiments 58-73, wherein the receptor comprises an APP (amyloid precursor protein) family member.

Embodiment 77. The method of any one of embodiments 58-73, wherein the receptor comprises an MHC protein.

Embodiment 78. The method of any one of embodiments 58-73, wherein the receptor comprises a GPCR.

Embodiment 79. The method of any one of embodiments 58-73, wherein the receptor comprises an adhesion protein.

Embodiment 80. The method of any one of embodiments 58-73, wherein the receptor comprises a tyrosine phosphatase.

Embodiment 81. The method of any one of embodiments 58-73, wherein the receptor comprises an integrin.

Embodiment 82. The method of any one of embodiments 58-73, wherein the receptor comprises an ion channel.

Embodiment 83. The method of any one of embodiments 58-73, wherein the receptor comprises a mineral transporter.

Embodiment 84. The method of any one of embodiments 58-73, wherein the receptor comprises a nutrient transporter.

Embodiment 85. The method of any one of embodiments 58-73, wherein the receptor comprises an ATPase.

Embodiment 86. The method of any one of embodiments 58-73, wherein the receptor comprises APLP2.

Embodiment 87. The method of any one of embodiments 58-73, wherein the receptor comprises PKP4.

Embodiment 88. The method of any one of embodiments 58-73, wherein the receptor comprises HLA-A.

Embodiment 89. The method of any one of embodiments 58-73, wherein the receptor comprises CD41.

Embodiment 90. The method of any one of embodiments 58-73, wherein the receptor comprises CD97.

Embodiment 91. The method of any one of embodiments 58-73, wherein the receptor comprises Integrin αVβ5.

Embodiment 92. The method of any one of embodiments 58-73, wherein the receptor comprises PI4K2A.

Embodiment 93. The method of any one of embodiments 58-73, wherein the receptor comprises TMEM120A.

Embodiment 94. The method of any one of embodiments 58-73, wherein the receptor comprises PTK7A.

Embodiment 95. The method of any one of embodiments 58-73, wherein the receptor comprises Ceruloplasmin.

Embodiment 96. The method of any one of embodiments 58-73, wherein the receptor comprises PTPRF.

Embodiment 97. The method of any one of embodiments 58-73, wherein the receptor comprises SLC16A1.

Embodiment 98. The method of any one of embodiments 58-73, wherein the receptor comprises SLC7A5.

Embodiment 99. The method of any one of embodiments 58-73, wherein the receptor comprises ATP1B1.

Embodiment 100. The method of any one of embodiments 58-73, wherein the receptor comprises ATP2B4.

Embodiment 101. The method of any one of embodiments 58-73, wherein the receptor comprises P142A.

Embodiment 102. The method of any one of embodiments 58-101, wherein the target cells comprise cancer cells.

Embodiment 103. The method of any one of embodiments 58-102, wherein the target cells comprise leukocytes.

Embodiment 104. The method of any one of embodiments 58-103, wherein the target cells comprise lymphocytes.

Embodiment 105. The method of any one of embodiments 58-104, wherein the target cells comprise B lymphocytes.

Embodiment 106. The method of any one of embodiments 58-105, wherein the target cells comprise plasma cells.

Embodiment 107. The method of any one of embodiments 58-106, wherein the target cells comprise multiple myeloma cells.

Embodiment 108. The method of any one of embodiments 58-102, wherein the target cells comprise pancreatic cells.

Embodiment 109. The method of any one of embodiments 58-102, wherein the target cells comprise pancreatic cancer cells.

Embodiment 110. The method of any one of embodiments 58-102, wherein the target cells comprise gastrointestinal cells.

Embodiment 111. The method of any one of embodiments 58-102, wherein the target cells comprise colorectal cancer cells.

Embodiment 112. A method comprising determining that a biological sample from a subject expresses a receptor that is associated with uptake of ctDNA at a level that is associated with the uptake of the ctDNA.

Embodiment 113. The method of embodiment 112, further comprising administering to the subject a therapeutically-effective amount of a therapeutic agent, wherein the administering is based at least in part on the determining that the biological sample from the subject expresses the receptor that is associated with the uptake of the ctDNA at the level that is associated with the uptake of the ctDNA.

Embodiment 114. The method of embodiment 112 or embodiment 113, wherein the determining comprises contacting the biological sample with an agent that binds to the receptor.

Embodiment 115. The method of embodiment 114, wherein the agent that binds to the receptor comprises an antibody or an antigen-binding fragment thereof.

Embodiment 116. The method of any one of embodiments 112-115, wherein the determining comprises an immunohistochemistry assay.

Embodiment 117. The method of any one of embodiments 112-116, wherein the determining comprises quantifying a level the receptor at protein level.

Embodiment 118. The method of embodiment 112 or embodiment 113, wherein the determining comprises quantifying a level of an mRNA that encodes the receptor.

Embodiment 119. The method of embodiment 112 or embodiment 113, wherein the determining comprises contacting the biological sample with an antibody that binds to the receptor and contacting the biological sample with a detection agent that induces a detectable signal based on a presence of the antibody bound to the receptor.

Embodiment 120. The method of any one of embodiments 112-119, wherein the receptor comprises an armadillo-like protein.

Embodiment 121. The method of any one of embodiments 112-120, wherein the receptor comprises a p120(ctn)/plakophilin subfamily member.

Embodiment 122. The method of any one of embodiments 112-119, wherein the receptor comprises an APP (amyloid precursor protein) family member.

Embodiment 123. The method of any one of embodiments 112-119, wherein the receptor comprises an MHC protein.

Embodiment 124. The method of any one of embodiments 112-119 wherein the receptor comprises a GPCR.

Embodiment 125. The method of any one of embodiments 112-119, wherein the receptor comprises an adhesion protein.

Embodiment 126. The method of any one of embodiments 112-119, wherein the receptor comprises a tyrosine phosphatase.

Embodiment 127. The method of any one of embodiments 112-119, wherein the receptor comprises an integrin.

Embodiment 128. The method of any one of embodiments 112-119, wherein the receptor comprises an ion channel.

Embodiment 129. The method of any one of embodiments 112-119, wherein the receptor comprises a mineral transporter.

Embodiment 130. The method of any one of embodiments 112-119, wherein the receptor comprises a nutrient transporter.

Embodiment 131. The method of any one of embodiments 112-119, wherein the receptor comprises an ATPase.

Embodiment 132. The method of any one of embodiments 112-119, wherein the receptor comprises APLP2.

Embodiment 133. The method of any one of embodiments 112-119, wherein the receptor comprises PKP4.

Embodiment 134. The method of any one of embodiments 112-119, wherein the receptor comprises HLA-A.

Embodiment 135. The method of any one of embodiments 112-119, wherein the receptor comprises CD41.

Embodiment 136. The method of any one of embodiments 112-119, wherein the receptor comprises CD97.

Embodiment 137. The method of any one of embodiments 112-119, wherein the receptor comprises Integrin αVβ5.

Embodiment 138. The method of any one of embodiments 112-119, wherein the receptor comprises PI4K2A.

Embodiment 139. The method of any one of embodiments 112-119, wherein the receptor comprises TMEM120A.

Embodiment 140. The method of any one of embodiments 112-119, wherein the receptor comprises PTK7A.

Embodiment 141. The method of any one of embodiments 112-119, wherein the receptor comprises Ceruloplasmin.

Embodiment 142. The method of any one of embodiments 112-119, wherein the receptor comprises PTPRF.

Embodiment 143. The method of any one of embodiments 112-119, wherein the receptor comprises SLC16A1.

Embodiment 144. The method of any one of embodiments 112-119, wherein the receptor comprises SLC7A5.

Embodiment 145. The method of any one of embodiments 112-119, wherein the receptor comprises ATP1B1.

Embodiment 146. The method of any one of embodiments 112-119, wherein the receptor comprises ATP2B4.

Embodiment 147. The method of any one of embodiments 112-119, wherein the receptor comprises P142A.

Embodiment 148. A method of treating a condition in a subject in need thereof, the method comprising administering a therapeutically-effective amount of a therapeutic agent to the subject, wherein the administering is based at least in part on a result of an assay performed on a biological sample from the subject, wherein the assay determined that a receptor associated with uptake of ctDNA is expressed in the biological sample at a level that is associated with the uptake of the ctDNA.

Embodiment 149. The method of embodiment 148, wherein the assay comprises contacting the biological sample with an agent that binds to the receptor.

Embodiment 150. The method of embodiment 149, wherein the agent that binds to the receptor comprises an antibody or an antigen-binding fragment thereof.

Embodiment 151. The method of any one of embodiments 148-150, wherein the assay comprises an immunohistochemistry assay.

Embodiment 152. The method of any one of embodiments 148-150, wherein the assay comprises quantifying a level of the receptor at protein level.

Embodiment 153. The method of embodiment 148, wherein the assay comprises quantifying a level of an mRNA that encodes the receptor.

Embodiment 154. The method of embodiment 148, wherein the assay comprises contacting the biological sample with an antibody that binds to the receptor and contacting the biological sample with a detection agent that induces a detectable signal based on the presence of the antibody bound to the receptor.

Embodiment 155. The method of any one of embodiments 148-154, wherein the receptor comprises an armadillo-like protein.

Embodiment 156. The method of any one of embodiments 148-155, wherein the receptor comprises a p120(ctn)/plakophilin subfamily member.

Embodiment 157. The method of any one of embodiments 148-154, wherein the receptor comprises an APP (amyloid precursor protein) family member.

Embodiment 158. The method of any one of embodiments 148-154, wherein the receptor comprises an MHC protein.

Embodiment 159. The method of any one of embodiments 148-154, wherein the receptor comprises a GPCR.

Embodiment 160. The method of any one of embodiments 148-154, wherein the receptor comprises an adhesion protein.

Embodiment 161. The method of any one of embodiments 148-154, wherein the receptor comprises a tyrosine phosphatase.

Embodiment 162. The method of any one of embodiments 148-154, wherein the receptor comprises an integrin.

Embodiment 163. The method of any one of embodiments 148-154, wherein the receptor comprises an ion channel.

Embodiment 164. The method of any one of embodiments 148-154, wherein the receptor comprises a mineral transporter.

Embodiment 165. The method of any one of embodiments 148-154, wherein the receptor comprises a nutrient transporter.

Embodiment 166. The method of any one of embodiments 148-154, wherein the receptor comprises an ATPase.

Embodiment 167. The method of any one of embodiments 148-154, wherein the receptor comprises APLP2.

Embodiment 168. The method of any one of embodiments 148-154, wherein the receptor comprises PKP4.

Embodiment 169. The method of any one of embodiments 148-154, wherein the receptor comprises HLA-A.

Embodiment 170. The method of any one of embodiments 148-154, wherein the receptor comprises CD41.

Embodiment 171. The method of any one of embodiments 148-154, wherein the receptor comprises CD97.

Embodiment 172. The method of any one of embodiments 148-154, wherein the receptor comprises Integrin αVβ5.

Embodiment 173. The method of any one of embodiments 148-154, wherein the receptor comprises PI4K2A.

Embodiment 174. The method of any one of embodiments 148-154, wherein the receptor comprises TMEM120A.

Embodiment 175. The method of any one of embodiments 148-154, wherein the receptor comprises PTK7A.

Embodiment 176. The method of any one of embodiments 148-154, wherein the receptor comprises Ceruloplasmin.

Embodiment 177. The method of any one of embodiments 148-154, wherein the receptor comprises PTPRF.

Embodiment 178. The method of any one of embodiments 148-154, wherein the receptor comprises SLC16A1.

Embodiment 179. The method of any one of embodiments 148-154, wherein the receptor comprises SLC7A5.

Embodiment 180. The method of any one of embodiments 148-154, wherein the receptor comprises APLP2.

Embodiment 181. The method of any one of embodiments 148-154, wherein the receptor comprises ATP1B1.

Embodiment 182. The method of any one of embodiments 148-154, wherein the receptor comprises ATP2B4.

Embodiment 183. The method of any one of embodiments 148-154, wherein the receptor comprises P142A.

Embodiment 184. The method of any one of embodiments 148-183, wherein the therapeutic agent comprises an anti-cancer therapeutic.

Embodiment 185. The method of any one of embodiments 148-183, wherein the therapeutic agent comprises a cytotoxic anti-cancer drug.

Embodiment 186. The method of any one of embodiments 148-183, wherein the therapeutic agent comprises an antagonist of the receptor.

Embodiment 187. The method of any one of embodiments 148-185, wherein the therapeutic agent comprises a synthetic delivery system.

Embodiment 188. The method of embodiment 187, wherein the synthetic delivery system comprises a polynucleotide.

Embodiment 189. The method of embodiment 188, wherein the polynucleotide induces uptake of the synthetic delivery system or a component thereof by a target cell.

Embodiment 190. The method of embodiment 188 or embodiment 189, wherein the polynucleotide comprises a nucleic acid sequence identified in circulating tumor DNA (ctDNA) or a derivative thereof.

Embodiment 191. The method of any one of embodiments 188-190, wherein the polynucleotide comprises double stranded DNA.

Embodiment 192. The method of any one of embodiments 188-191, wherein the polynucleotide comprises a transposable element.

Embodiment 193. The method of embodiment 192, wherein the transposable element comprises a short interspersed nuclear elements (SINE) transposable element.

Embodiment 194. The method of embodiment 192, wherein the transposable element comprises a long interspersed nuclear elements (LINE) transposable element.

Embodiment 195. The method of embodiment 192, wherein the transposable element comprises an ERVL transposable element.

Embodiment 196. The method of embodiment 192, wherein the transposable element comprises an ERVK transposable element.

Embodiment 197. The method of embodiment 192, wherein the transposable element comprises an AluSp transposable element.

Embodiment 198. The method of embodiment 192, wherein the transposable element comprises a MER11C transposable element.

Embodiment 199. The method of embodiment 192, wherein the transposable element comprises an AluSx transposable element.

Embodiment 200. The method of embodiment 192, wherein the transposable element comprises an MTL1J transposable element.

Embodiment 201. The method of embodiment 192, wherein the transposable element comprises an AluSg2 transposable element.

Embodiment 202. The method of embodiment 192, wherein the transposable element comprises a THE1A transposable element.

Embodiment 203. The method of embodiment 192, wherein the transposable element comprises an AluJb transposable element.

Embodiment 204. The method of embodiment 192, wherein the transposable element comprises an MTL2B4 transposable element.

Embodiment 205. The method of embodiment 192, wherein the transposable element comprises an L2a transposable element.

Embodiment 206. The method of embodiment 192, wherein the transposable element comprises an MTL1J2 transposable element.

Embodiment 207. The method of embodiment 192, wherein the transposable element comprises a L1MB3 transposable element.

Embodiment 208. The method of embodiment 192, wherein the transposable element comprises a THE1C transposable element.

Embodiment 209. The method of embodiment 192, wherein the transposable element comprises an AluY transposable element.

Embodiment 210. The method of any one of embodiments 187-209, wherein the synthetic delivery system further comprises a transposon integration signal.

Embodiment 211. The method of any one of embodiments 187-210, wherein the synthetic delivery system further comprises a cargo.

Embodiment 212. The method of embodiment 211, wherein the cargo comprises a nucleic acid cargo.

Embodiment 213. The method of embodiment 212, wherein the nucleic acid cargo encodes a tumor suppressor protein.

Embodiment 214. The method of any one of embodiments 187-213, wherein the synthetic delivery system comprises a promoter.

Embodiment 215. The method of any one of embodiments 211-213, wherein the cargo comprises a cytotoxic cargo.

Embodiment 216. The method of any one of embodiments 148-215, wherein the ctDNA comprises a short interspersed nuclear elements (SINE) transposable element.

Embodiment 217. The method of any one of embodiments 148-215, wherein the ctDNA comprises a long interspersed nuclear elements (LINE) transposable element.

Embodiment 218. The method of any one of embodiments 148-215, wherein the ctDNA comprises an ERVL transposable element.

Embodiment 219. The method of any one of embodiments 148-215, wherein the ctDNA comprises an ERVK transposable element.

Embodiment 220. The method of any one of embodiments 148-215, wherein the ctDNA comprises an AluSp transposable element.

Embodiment 221. The method of any one of embodiments 148-215, wherein the ctDNA comprises a MER11C transposable element.

Embodiment 222. The method of any one of embodiments 112-221, wherein the biological sample comprises cancer cells.

Embodiment 223. The method of any one of embodiments 112-222, wherein the biological sample comprises a blood sample.

Embodiment 224. The method of any one of embodiments 112-222, wherein the biological sample comprises a tumor biopsy.

Embodiment 225. The method of any one of embodiments 148-224, wherein the condition is a cancer.

Embodiment 226. The method of any one of embodiments 148-224, wherein the condition is multiple myeloma.

Embodiment 227. The method of any one of embodiments 148-224, wherein the condition is pancreatic cancer.

Embodiment 228. The method of any one of embodiments 148-224, wherein the condition is colorectal cancer.

Embodiment 229. The method of any one of embodiments 112-228, wherein the subject is a human.

Embodiment 230. The method of any one of embodiments 112-228, wherein the subject is a mammal.

Embodiment 231. A method of identifying a receptor that mediates uptake of a cargo by a target cell, the method comprising: (a) isolating ctDNA or a derivative thereof that is bound to the receptor or a fragment thereof; and (b) identifying the receptor.

Embodiment 232. The method of embodiment 231, further comprising contacting the ctDNA or derivative thereof with cell lysate generated from the target cell.

Embodiment 233. The method of embodiment 231 or embodiment 232, wherein the isolating the ctDNA or derivative thereof comprises a pulldown assay.

Embodiment 234. The method of any one of embodiments 231-233, wherein the identifying the receptor comprises a mass spectrometry assay.

Embodiment 235. The method of any one of embodiments 231-234, further comprising contacting a population of cells with an antibody that binds to the receptor and determining an effect of the antibody binding to the receptor on cellular uptake or nuclear accumulation of the ctDNA or derivative thereof.

Embodiment 236. A method of delivering a cargo to a target cell, the method comprising contacting a receptor on the target cell with a synthetic delivery system that comprises a polynucleotide, thereby inducing uptake of the cargo by the target cell, wherein: (a) the polynucleotide comprises a first nucleic acid sequence identified in circulating tumor DNA (ctDNA) from a substantially similar cell type as the target cell; and (b) the receptor comprises an armadillo-like protein, an APP (amyloid precursor protein) family member, a p120(ctn)/plakophilin subfamily member, an MHC protein, a GPCR, an adhesion protein, a tyrosine phosphatase, an integrin, an ion channel, a mineral transporter, a nutrient transporter, or an ATPase.

Embodiment 237. The method of embodiment 236, wherein the receptor comprises the MHC protein.

Embodiment 238. The method of embodiment 236, wherein the receptor comprises the GPCR.

Embodiment 239. The method of embodiment 236, wherein the receptor comprises the adhesion protein.

Embodiment 240. The method of embodiment 236, wherein the receptor comprises the tyrosine phosphatase.

Embodiment 241. The method of embodiment 236, wherein the receptor comprises the integrin.

Embodiment 242. The method of embodiment 236, wherein the receptor comprises the ion channel.

Embodiment 243. The method of embodiment 236, wherein the receptor comprises the mineral transporter.

Embodiment 244. The method of embodiment 236, wherein the receptor comprises the nutrient transporter.

Embodiment 245. The method of embodiment 236, wherein the receptor comprises the ATPase.

Embodiment 246. The method of embodiment 236, wherein the receptor comprises HLA-A.

Embodiment 247. The method of embodiment 236, wherein the receptor comprises CD41.

Embodiment 248. The method of embodiment 236, wherein the receptor comprises CD97.

Embodiment 249. The method of embodiment 236, wherein the receptor comprises Integrin αVβ5.

Embodiment 250. The method of embodiment 236, wherein the receptor comprises PI4K2A.

Embodiment 251. The method of embodiment 236, wherein the receptor comprises TMEM120A.

Embodiment 252. The method of embodiment 236, wherein the receptor comprises PTK7A.

Embodiment 253. The method of embodiment 236, wherein the receptor comprises Ceruloplasmin.

Embodiment 254. The method of embodiment 236, wherein the receptor comprises PTPRF.

Embodiment 255. The method of embodiment 236, wherein the receptor comprises SLC16A1.

Embodiment 256. The method of embodiment 236, wherein the receptor comprises SLC7A5.

Embodiment 257. The method of embodiment 236, wherein the receptor comprises APLP2.

Embodiment 258. The method of embodiment 236, wherein the receptor comprises ATP1BL.

Embodiment 259. The method of embodiment 236, wherein the receptor comprises ATP2B4.

Embodiment 260. The method of embodiment 236, wherein the receptor comprises P142A.

Embodiment 261. The method of embodiment 236, wherein the receptor comprises the armadillo-like protein.

Embodiment 262. The method of embodiment 236, wherein the receptor comprises the APP family member.

Embodiment 263. The method of embodiment 236, wherein the receptor comprises the p120(ctn)/plakophilin subfamily member.

Embodiment 264. The method of embodiment 236, wherein the receptor comprises PKP4.

Embodiment 265. The method of any one of embodiments 236-264, wherein an anti-dsDNA antibody inhibits uptake of the cargo by the target cell at least 10% as determined by a confocal fluorescence microscopy assay.

Embodiment 266. The method of any one of embodiments 236-264, wherein an antibody that binds to the receptor reduces uptake of the cargo by the target cell at least 10% as determined by a confocal fluorescence microscopy assay.

Embodiment 267. The method of any one of embodiments 236-264, wherein the first nucleic acid sequence is in a 3′ region of the polynucleotide.

Embodiment 268. The method of any one of embodiments 236-267, wherein the polynucleotide further comprises a second nucleic acid sequence identified in the ctDNA from the substantially similar cell type as the target cell.

Embodiment 269. The method of embodiment 268, wherein the second nucleic acid sequence is in a 5′ region of the polynucleotide.

Embodiment 270. The method of any one of embodiments 236-269, wherein the first nucleic acid sequence is in a 5′ region of the polynucleotide.

Embodiment 271. The method of any one of embodiments 236-270, wherein the polynucleotide further comprises a second nucleic acid sequence identified in the ctDNA from the substantially similar cell type as the target cell.

Embodiment 272. The method of embodiment 271, wherein the second nucleic acid sequence is in a 3′ region of the polynucleotide.

Embodiment 273. The method of any one of embodiments 236-272, wherein the polynucleotide is double stranded DNA.

Embodiment 274. The method of any one of embodiments 236-273, wherein the synthetic delivery system does not utilize a viral vector, a nanoparticle, a lipid nanoparticle, a liposome, an exosome, a dendrimer, a gene gun, or electroporation.

Embodiment 275. The method of any one of embodiments 236-274, wherein the cargo is delivered to a nucleus of the target cell.

Embodiment 276. The method of any one of embodiments 236-275, wherein the synthetic delivery system further comprises a transposon integration signal.

Embodiment 277. The method of any one of embodiments 236-276, wherein the cargo comprises a nucleic acid cargo.

Embodiment 278. The method of embodiment 277, wherein the nucleic acid cargo is integrated into the target cell's genome.

Embodiment 279. The method of embodiment 278, wherein an anti-dsDNA antibody reduces integration of the nucleic acid cargo into the target cell's genome at least 10% as determined by a metaphase chromosome spread assay or a confocal fluorescence microscopy assay.

Embodiment 280. The method of embodiment 277, wherein an antibody that binds to the receptor reduces integration of the nucleic acid cargo into the target cell's genome at least 10% as determined by a metaphase chromosome spread assay, sequencing, or a confocal fluorescence microscopy assay.

Embodiment 281. The method of any one of embodiments 236-280, wherein the target cell is a leukocyte.

Embodiment 282. The method of any one of embodiments 236-280, wherein the target cell is a myeloid cell.

Embodiment 282. The method of any one of embodiments 236-280, wherein the target cell is a lymphoid cell.

Embodiment 282. The method of any one of embodiments 236-280, wherein the target cell is a plasma cell.

Embodiment 283. The method of any one of embodiments 236-280, wherein the target cell is a multiple myeloma cell.

Embodiment 284. The method of any one of embodiments 236-280, wherein the target cell is a pancreatic cell.

Embodiment 285. The method of any one of embodiments 236-280, wherein the target cell is a pancreatic cancer cell.

Embodiment 286. The method of any one of embodiments 236-280, wherein the target cell is a gastrointestinal cell.

Embodiment 287. The method of any one of embodiments 236-280, wherein the target cell is a colorectal cancer cell.

### EXAMPLES

### Example 1: Involvement of Membrane Proteins in Cellular Uptake of Circulating Tumor DNA (ctDNA)

This example demonstrates that cleavage of surface-exposed membrane proteins by trypsin reduces cellular uptake of circulating tumor DNA (ctDNA).

Multiple myeloma (MM1s) cells, pancreatic cancer (MIA) cells, and colon cancer (HCT116) cells were cultured with CY5 labelled-ctDNA from patients with corresponding types of cancer (e.g., multiple myeloma, pancreatic cancer, and colon cancer, respectively). The cells were cultured with or without trypsin treatment to cleave cell surface-exposed membrane proteins. CY5 ctDNA fluorescence was measured by flow cytometry.

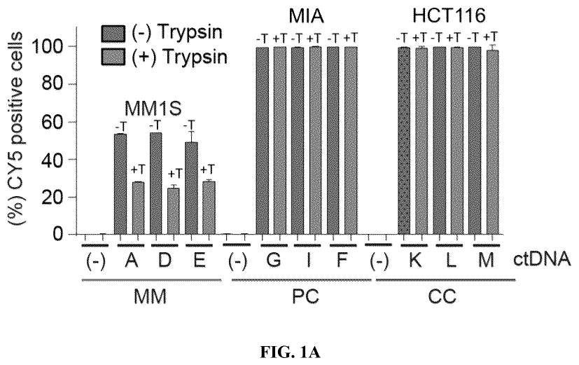

The addition of trypsin reduced the proportion of multiple myeloma cells that were positive for CY5 ctDNA fluorescence from about 52-54% to about 20-30% (FIG. 1A, quantifying the percent positive cells upon incubation with ctDNA from three multiple myeloma patients (A, D, E), with (+T) or without (−T) trypsin treatment). For example, in a representative experiment, trypsin treatment reduced the percent of MM1s cells that were positive for ctDNA signal from about 54.2% to about 29.1% (FIG. 1B, top panels, showing representative scatterplots of cells in the various treatment conditions). These results suggested that ctDNA incorporation is mediated by interaction of ctDNA with a cell surface receptor protein.

Almost all (˜99%) pancreatic cancer cells and colon cancer cells were positive for ctDNA, with or without trypsin treatment (FIG. 1A, FIG. 1B).

### Example 2: Anti-Double-Stranded DNA Antibodies Reduce Nuclear Localization of ctDNA

This example demonstrates that anti-double-stranded DNA (anti-dsDNA) antibodies reduce nuclear localization of ctDNA.

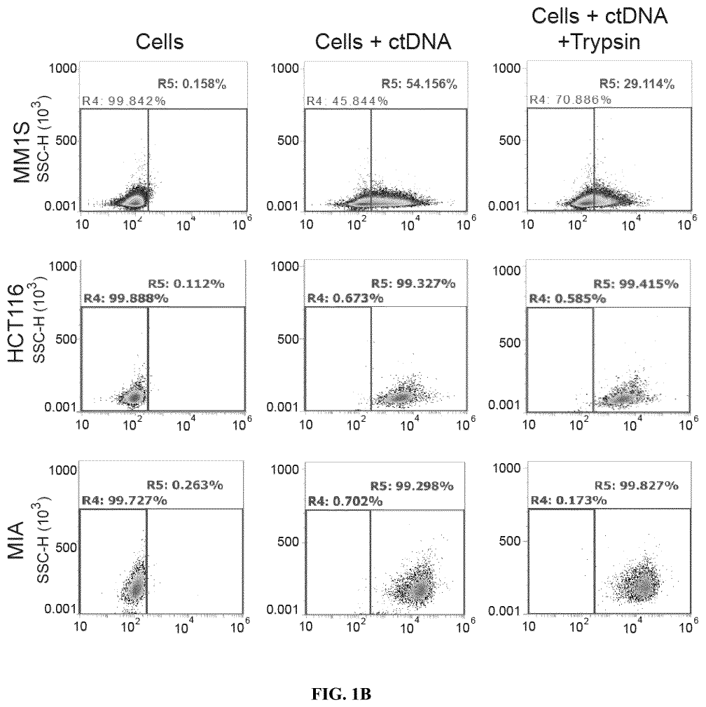

ctDNA from multiple myeloma or pancreatic cancer patients was incubated with 1 g/mL of anti-dsDNA antibodies. The anti-dsDNA antibodies were from subjects with systemic lupus erythematosus (subjects SLE1, SLE2). Control ctDNA samples were incubated with IgG isotype, or no antibody. After 24 hours of incubating the ctDNA with the anti-dsDNA antibodies, the ctDNA was added to cultures of cell lines of the same cell type as the ctDNA's tissue of origin, multiple myeloma (MM1s) cells or pancreatic cancer (MIA) cells. 24 hours later, samples were imaged and the nuclear intensity of ctDNA determined. Pre-treatment of ctDNA with anti-dsDNA antibodies significantly reduced nuclear localization of the ctDNA relative to IgG isotype control or PBS (FIG. 2; ctDNA nuclear intensity: 3.23-4.2 MM and 2.06-2.8 PC; IgG isotype control 16.5 MM ctDNA and 25.6 PC; or PBS 7.9 MM and 14 PC; p<0.0001).

### Example 3: Identification of Cell Membrane Proteins that Bind ctDNA

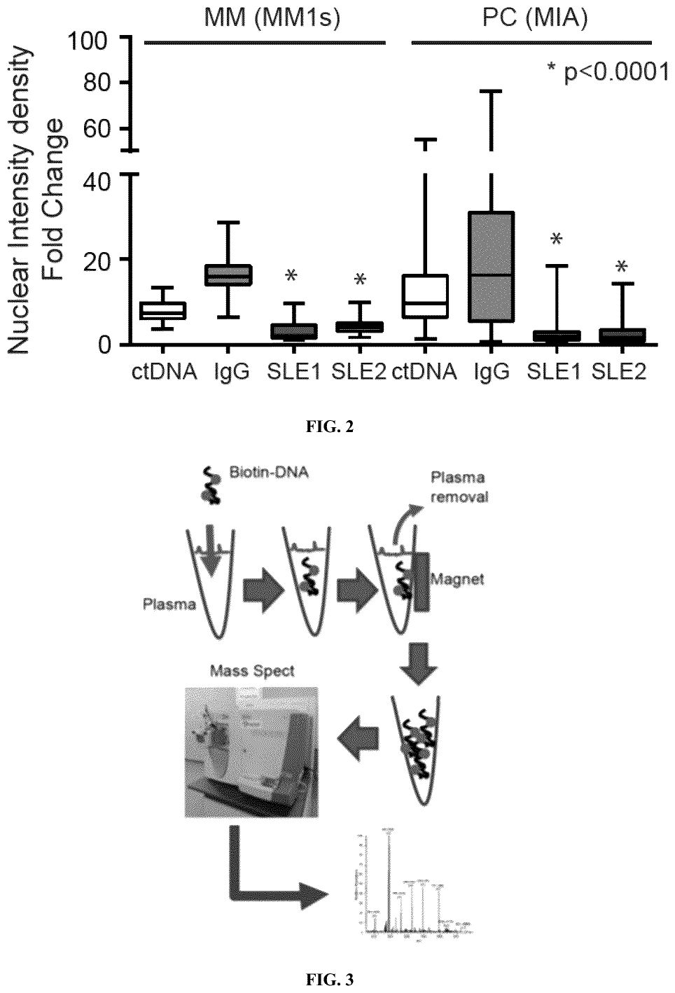

To evaluate proteins that can potentially function as ctDNA recognizing receptors (e.g., that mediate internalization of ctDNA into cells), pull down and mass spectrometry assays were conducted (FIG. 3).

ctDNA was extracted from plasma of patients with multiple myeloma, pancreatic cancer, or colon cancer using a QIAamp Circulating Nucleic Acid Kit. For control DNA, a CMV-GFP vector was linearized. ctDNA and control GFP DNA were labeled with biotin using a Biotin Label IT® Nucleic Acid Labeling kit. A biotin-only control was used to identify false positives.

A first co-incubation was conducted to bind ctDNA to plasma proteins. The ctDNA samples were incubated with plasma from the same cancer type as the ctDNA. In some experimental conditions, the plasma was pre-treated to deplete the most common plasma proteins using a commercial kit to enhance signal from less common proteins. Each of the following were incubated for 24 hours: (a) biotin and plasma; (b) biotinylated GFP DNA and plasma; (c) biotinylated ctDNA and plasma; (d) biotinylated ctDNA and plasma depleted of the most common plasma proteins; and (e) biotinylated ctDNA.

After incubating for 24 hours, a first pulldown was performed using streptavidin magnetic beads to collect biotin-labeled DNA and plasma proteins bound thereto.

A second co-incubation was then conducted to bind the output of the first pulldown to cell lysates. Three cell lines of different tumor types were used: MI1s (multiple myeloma) cells, MIA (pancreatic cancer) cells, and HCT116 (colon cancer) cells. Lysates were obtained from 50 million cells of each cell line, and the outputs of the first pulldown were incubated with the cell lysates for 24 hours. The ctDNA samples were incubated with lysates from the same cell type as the ctDNA.

A second pulldown was then performed to obtain samples with ctDNA that bound to proteins in the cell lysate. The products obtained were subjected to Fusion Orbitrap mass spectrometry to identify proteins.

Enrichment of proteins that bound to ctDNA was determined based on comparing the ctDNA conditions against the biotin alone and biotinylated GFP DNA conditions.

Two methods of analysis were performed. In a first analysis, data were analyzed after quantile normalization and MAxQuant software analysis was used for linear modeling. In a second analysis, proteins selected met the following criteria: (a) at least three-fold increase compared to biotin alone and biotinylated GFP DNA conditions, and (b) enriched across all tumor types in similar experimental conditions.

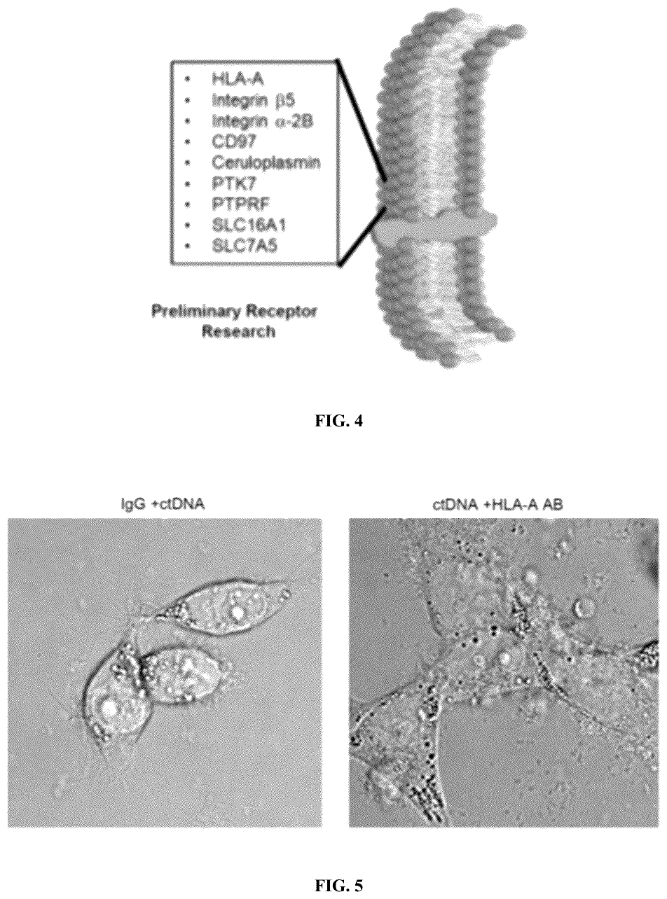

Illustrative proteins identified as candidates that bind to ctDNA are provided in Table 1, and FIG. 4. Non-limiting examples of proteins identified as candidates that bind to ctDNA include HLA-A, CD41 (ITGA21B; Integrin alpha 2b), CD97, Integrin β5 (ITGB5), Integrin αVβ5, PI4K2A, TMEM12A, PTK7A, ceruloplasmin, PTPRF, SLC16A1, SLC7A5, ATP1B1, ATP2B34, and APLP2.

Table 3: illustrative membrane bound proteins identified as interacting with ctDNA,

TABLE 3

| Ratios from raw data | | | Quantile norm/ MaxQuant | | | | |
|---|---|---|---|---|---|---|---|
| | depleted | | | depleted | | | |
| plasma + | plasma + | | plasma + | plasma + | | Accession/ | |
| lysate | lysate | lysate | lysate | lysate | lysate | Gene Symbol | Description |
| X | X | X | X | X | X | A0A0G2JI | HLA class I |
| | | | | | | F2/ | histocompatibility |
| | | | | | | HLA-A | antigen, A alpha |
| | | | | | | | chain |
| | X | X | | | | P48960/ | CD97 antigen |
| | | | | | | CD97 | |
| | X | | | | | P00450/CP | Ceruloplasmin |
| | X | X | | | | Q13308/ | Inactive tyrosine- |
| | | | | | | PTK7 | protein kinase 7 |
| X | X | | X | X | X | P10586/PT | Receptor-type |
| | | | | | | PRF | tyrosine-protein |
| | | | | | | | phosphatase F |
| | X | | | | | P53985/ | Monocarboxylate |
| | | | | | | SLC16A1 | transporter 1 |
| | | X | | | | Q01650/ | Large neutral amino |
| | | | | | | SLC7A5 | acids transporter |
| | | | | | | | small subunit 1 |
| | | X | | X | X | Q9BXJ8/ | Transmembrane |
| | | | | | | TMEM120A | protein 120A |
| | | X | | | | P18084/ | Integrin beta-5 |
| | | | | | | ITGB5 | OS = Homo sapiens |
| | | | | | | | OX = 9606 |
| | | | | | | | GN = ITGB5 PE = 1 |
| | | | | | | | SV = 1 |
| | | X | | | | P08514/ | Integrin alpha-IIb |
| | | | | | | ITGA2B | |
| | | | | | | (CD41) | |
| | | X | | | | P05026/ | Sodium/potassium- |
| | | | | | | ATP1B1 | transporting ATPase |
| | | | | | | | subunit beta-1 |
| | | X | | | | P23634/ | Plasma membrane |
| | | | | | | ATP2B4 | calcium-transporting |

The identified proteins included proteins involved in immune recognition and proteins with DNA binding capacity. HLA-A was the top protein identified and was present in all methodological analyses and in all experimental conditions. APLP2, which can interact with HLA-A during antigen recognition, was also identified.

### Example 4: Anti-HLA-A Antibody Reduces Nuclear Localization of ctDNA

This example demonstrates that an antibody that binds to HLA-A can reduce nuclear localization of ctDNA, suggesting that HLA-A can be involved in uptake of ctDNA.

5×10⁴ PANC-1 (pancreatic cancer) cells per replicate were pre-incubated for 4 hours with 1 μg/mL of anti-HLA-A antibody or IgG isotype control. Rhodamine-labeled ctDNA was then added at 1 μg/mL. After allowing time for uptake, cells were fixed by adding 4% paraformaldehyde for 15 minutes. Slides were washed with PBS twice, then cells were counterstained with 4, 6-diamidino-2-phenylindole (DAPI) for 10 minutes for nuclear detection. Images were acquired using Leica SP8 LIGHTING confocal microscope.

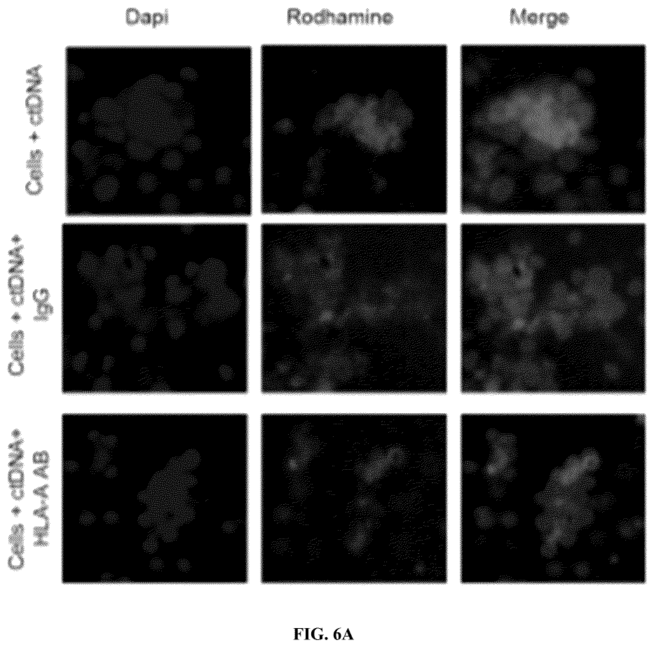

The anti-HLA-A antibody reduced ctDNA uptake and nuclear localization compared to the IgG isotype antibody (FIG. 5), suggesting that HLA-A can be involved in cellular uptake of ctDNA.

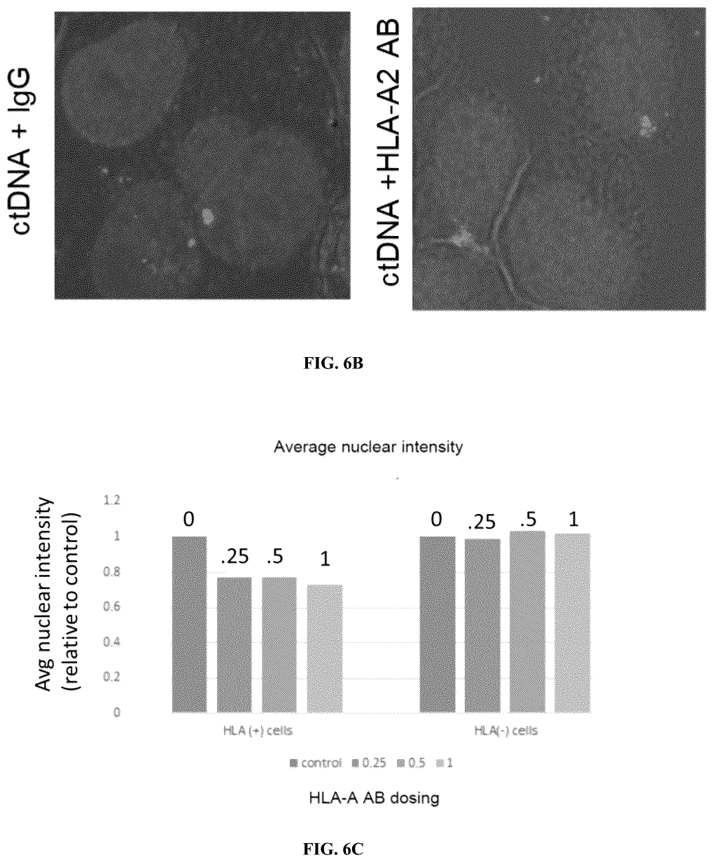

In an additional experiment, 5×10⁴ MM1s cells per replicate were seeded in a 96 well black clear bottom plate and pre-treated for 4 hours with titrating doses of anti-HLA-A (e.g., anti-HLA-A2) antibody or IgG isotype control (doses: 0, 0.25, 0.5, or 1 μg/mL), after which rhodamine-labeled ctDNA was added to the culture wells at 1 μg/mL and incubated for 24 hours. 30 minutes prior to the end of incubation period, cells were stained with Hoechst dye (5 μg/mL final) for nuclear detection, and the plate was scanned using an ImageXpress 5000A Automated Acquisition and Analysis System. Treatment with the anti-HLA-A antibody appeared to reduce internalization of ctDNA (FIG. 6A and FIG. 6B).

In some wells, cells were fixed and stained with a fluorescent secondary antibody to stain HLA-A-expressing cells and differentiate them from cells that were HLA-A negative. Pre-incubation with the anti-HLA-A antibody reduced nuclear intensity of the rhodamine-labeled ctDNA for cells that expressed HLA-A (FIG. 6C). The numbers above the columns indicate the concentration of the anti-HLA-A antibody (0, 0.25, 0.5, or 1 μg/mL).

### Example 5: Inhibition of ctDNA Nuclear Localization by Antibodies

The ability of antibodies to block ctDNA uptake and nuclear localization was evaluated.

MMI1s multiple myeloma cells were seeded at 5×10⁴ cells in mL per replicate, and pre-incubated with 1 g/mL of antibody for 4 hours prior to adding rhodamine-labelled or CY5-labelled ctDNA (1 g/mL). Cells were cultured with the ctDNA for 24 hours, then fixed. In some conditions, the fixed cells were treated with a AF488-labelled secondary antibody to stain cells with antibody bound to the candidate receptor and differentiate the stained cells from cells without antibody bound. Images were taken using Lattice light-sheet microscopy.

Adherent cells were grown on coverslips prior to processing. After cells were attached to slides, slides were washed with PBS twice, and the cells were then counterstained with 4, 6-diamidino-2-phenylindole (DAPI) for nuclear detection. For live-cell imaging, the plasma membrane was labeled following the cellLight Plasma Membrane-green fluorescent protein (GFP), Bacman 2.0 protocol. Images were acquired using a Leica SP8 LIGHTING confocal microscope.

Lattice light-sheet microscopy was used to obtain live image and movie acquisition. Images were acquired using a 3i v1 Lattice Light Sheet microscope in sample scanning mode, with Δs of 0.8 μm and 71 steps, and a 20 μm x-dither scan of the lattice pattern created with a 0.550 outer NA/0.500 inner NA annuli. Volume data were collected using a Hamamatsu ORCA-Flash 4.0 v2 via a Semrock FF01-446/523/600/677 blocking filter for both 488 nm and 560 nm laser channels (5% and 10% power respectively) every 3 mins for 1-2 hours. Raw data were deskewed using 3i SlideBook 6 software to create correctly orientation volumetric data. The 3D visualization, surfaces and movies were created in Bitplane Imaris 9. Isosurface settings were user selected for each dataset to efficiently represent signal boundaries.

For quantification of the nuclear localization of labelled ctDNA, 10 images were obtained per sample in fields with a minimum of 10 cells. Volumetric data sets were acquired using a Leica SP8 confocal microscope. All data were acquired with the same x, y, and z sampling, and with the same xy zoom. Z-stack total heights were varied to encapsulate the thickness of the randomly selected field of view. Data were analyzed using Fiji 24, ilastik 25, and Matlab computational software. A Fiji macro was used to convert raw.lif files as required; ilastik machine learning models were trained and then applied to classify specific nuclear morphologies; and Matlab was used to process resulting probability maps and quantify labelled ctDNA signal within the nuclei. Nuclear intensity fold change values were calculated by measuring the nuclear intensity produced by the labeled ctDNA over the background intensity of the nuclear signal in control cells.

Statistical results were analyzed by comparing each measurement with control ctDNA and ctDNA+IgG coculture conditions. Student's T test was used for measuring statistical significance.

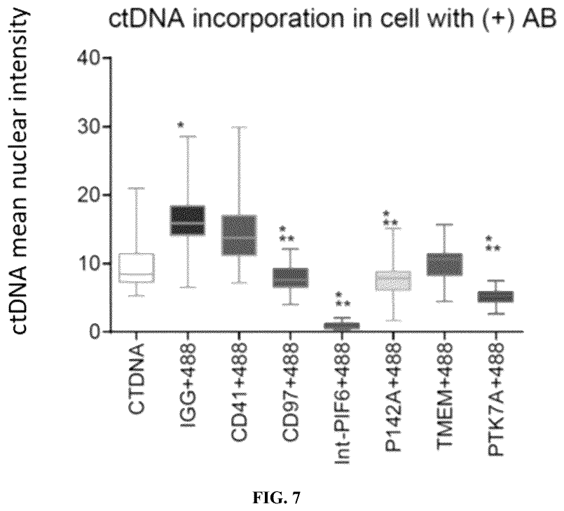

Of cells that stained positive for the anti-receptor antibody, reduced ctDNA mean nuclear intensity was observed for cells treated with anti-CD97, Integrin αVβ5 (labelled as Int-P1F6 for the antibody clone), anti-P142A, or anti-PTK7A antibodies (FIG. 7).

### Example 6: Detection of Multiple Myeloma ctDNA-Receptor Binding Via Modified Amplified Luminescent Proximity Homogeneous Assay (ALPHA)

To evaluate ctDNA binding to proteins that can potentially function as ctDNA recognizing receptors (e.g., that mediate internalization of ctDNA into cells) in multiple myeloma, an Amplified Luminescent Proximity Homogeneous Assay (ALPHA) was performed. Membrane lysates were extracted from 5×10⁷ cells from multiple myeloma cell lines (e.g., MI1a, OPM1, RPMI, JK6L) and non-myeloma cell lines (e.g., MIA, HTC116, A549, MDA-MD-468). ctDNA from a multiple myeloma individual was labeled with biotin. Pre-cleared membrane lysates (2.5 g/mL) were incubated for 1 hour with biotinylated-ctDNA (2 g/mL). A primary antibody (100 nM) (e.g., targeting a ctDNA-receptor candidate) was added to the mixture for 1 hour. The mixture was incubated for an additional hour with streptavidin-coated donor beads and protein A-acceptor beads. Next, luminescence was measured with an ALPHA-capable plate reader. IgG antibodies and biotin were used as controls. The luminescence ratio was measured between an antibody targeting a protein of interest and the biotin control signal. An antibody that increases the IgG/biotin ratio by more than threefold was identified as a protein with an active interaction with ctDNA.

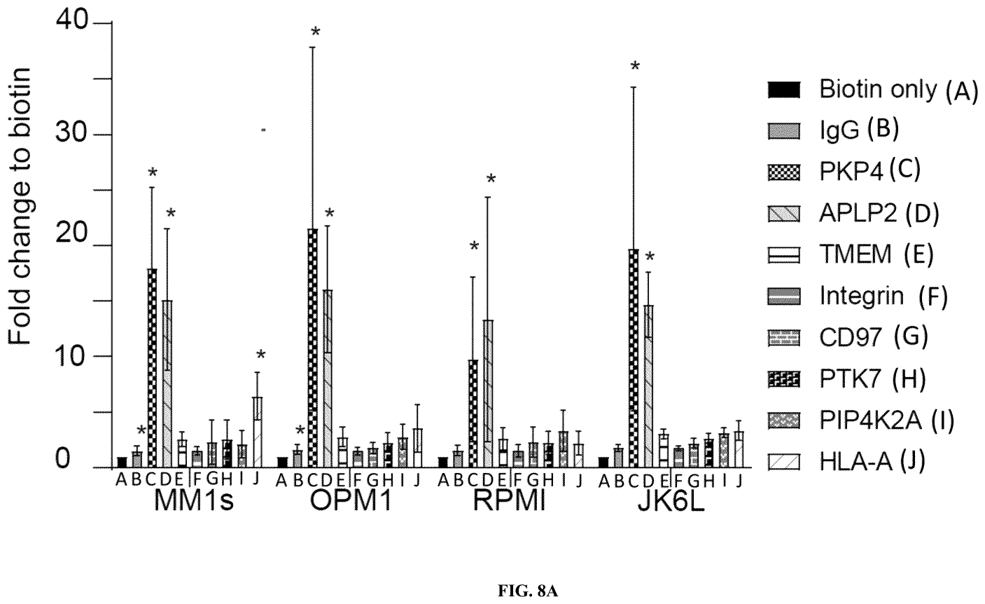

ALPHA revealed that PKP4 and APLP2 obtained from all multiple myeloma cell lines interacted with multiple myeloma ctDNA, as did HLA-A taken from MMIs cells (FIG. 8A). The interaction with multiple myeloma ctDNA was lost when these proteins were extracted from non-myeloma cell lines (FIG. 8B).

These data support a role of PKP4, APLP2, and/or HLA-A in binding and/or mediating uptake of multiple myeloma ctDNA by multiple myeloma cells.

### Example 7: Competitive Inhibition of ctDNA Uptake

This example demonstrates competitive inhibition of ctDNA uptake by other ctDNA, or by a synthetic delivery system polynucleotide, suggesting a shared receptor, binding/capture mechanism, and/or internalization mechanism.

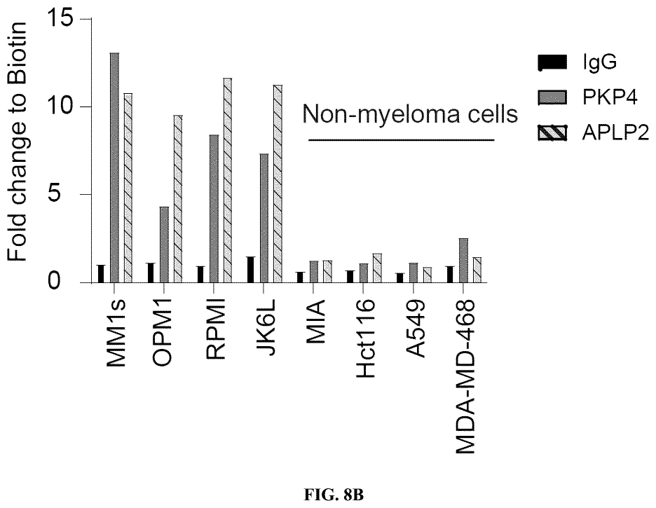

Multiple myeloma cells (e.g., MMIs cells) were cultured with unlabeled DNA at 0, 25, 50, 100, 200, 400, or 800 ng doses (unlabeled control DNA, unlabeled ctDNA, or an unlabeled synthetic delivery system polynucleotide comprising a recognition sequence (MM-ZC)). After one hour in culture with the unlabeled DNA, fluorescently labeled DNA was added (CY5 labeled control DNA, CY5 labeled ctDNA, or CY5 labeled synthetic delivery system polynucleotide comprising a recognition sequence (MM-ZC)). Cells were incubated for two hours to allow for binding and uptake of the labelled DNA, then analyzed by flow cytometry. The unlabeled ctDNA or synthetic delivery system polynucleotide inhibited uptake of labeled ctDNA or synthetic delivery system in a dose-dependent manner, as shown by a reduced percentage of cells positive for CY5 signal (FIG. 9).

These data are consistent with receptor-mediated capture of ctDNA and the synthetic delivery system polynucleotide, and suggest that the ctDNA and synthetic delivery system polynucleotide can share a receptor, binding/capture mechanism, and/or internalization mechanism. The same effect was not observed for control DNA.

These data also demonstrate reduced uptake of ctDNA by target cells upon treatment (e.g., contacting) with a DNA antagonist (e.g., synthetic delivery system polynucleotide, or ctDNA).

### Example 8: Colocalization of ctDNA and Receptors

This example demonstrates colocalization of ctDNA with receptors associated with the uptake of ctDNA.

MM1s cells were cultured for four hours with rhodamine-labeled ctDNA from multiple myeloma patients, then fixed. After fixation, immunofluorescence staining was conducted with primary antibodies specific for APLP2, PKP4, or HLA-A, and an Alexa fluor 488 labelled secondary antibody for detection.

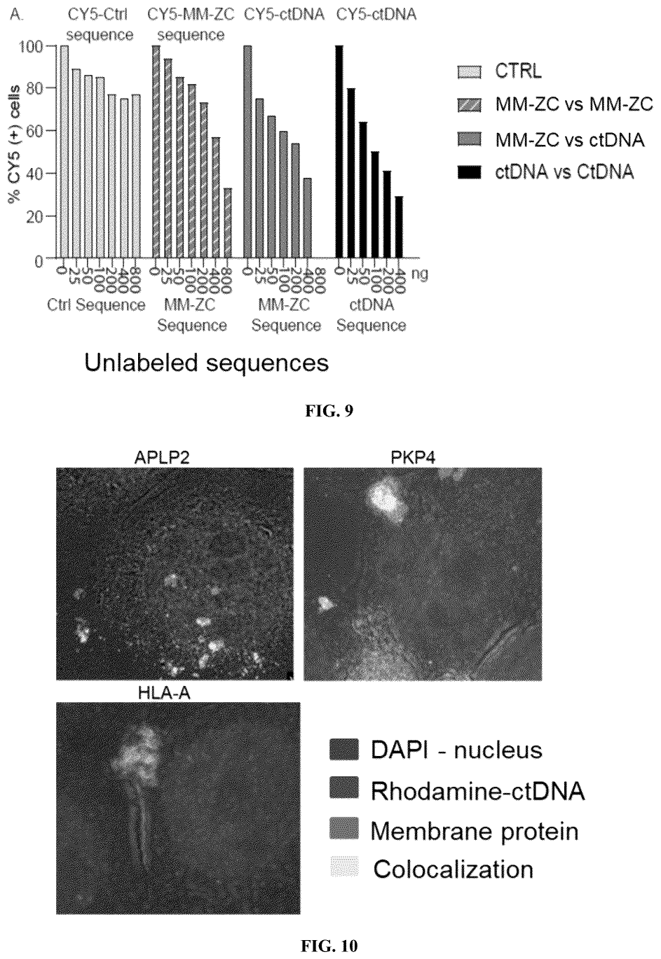

Fluorescence microscopy demonstrated colocalization of ctDNA with APLP2, PKP4, and HLA-A at the cell membrane (FIG. 10).

### Example 9: Detection of Multiple Origin ctDNA-Receptor Binding Via Modified Amplified Luminescent Proximity Homogeneous Assay (ALPHA)

Amplified Luminescent Proximity Homogeneous Assay (ALPHA) is performed to evaluate proteins that can potentially function as ctDNA recognizing receptors (e.g., that mediate internalization of ctDNA into cells). Membrane lysates are extracted from 5×10⁷ cells from various cancer cells (e.g., pancreatic cancer, colorectal cancer) and control cells, such as non-cancer cells, or cells of a different cancer type. ctDNA from an individual with cancer (e.g., pancreatic cancer, colorectal cancer) is labeled with biotin. Precleared membrane lysates (2.5 g/mL) are incubated for 1 hour with biotinylated-ctDNA (2 g/mL). A primary antibody (100 nM) (e.g., targeting a ctDNA-receptor candidate) is added to the mixture for 1 hour. The mixture is incubated for an additional hour with streptavidin-coated donor beads and protein A-acceptor beads. Next, luminescence is measured with an ALPHA-capable plate reader. IgG antibodies and biotin were used as controls. The luminescence ratio is measured between an antibody targeting a protein of interest and the biotin control signal. An antibody that increases the IgG/biotin ratio by more than threefold is identified as a protein with an active interaction with ctDNA.

### Example 10: Competitive Inhibition of ctDNA Uptake

This example demonstrates evaluation of competitive inhibition of ctDNA uptake by other ctDNA, or by a synthetic delivery system polynucleotide. Results of such an assay can suggest a shared receptor, binding/capture mechanism, and/or internalization mechanism.

Cells of suitable types (e.g., pancreatic cancer cells or cell lines, colorectal cancer cells or cell lines) are cultured with unlabeled DNA at 0, 25, 50, 100, 200, 400, or 800 ng doses (unlabeled control DNA, unlabeled ctDNA, or an unlabeled synthetic delivery system polynucleotide comprising a recognition sequence). After one hour in culture with the unlabeled DNA, fluorescently labeled DNA is added (e.g., CY5 labeled control DNA, CY5 labeled ctDNA, or CY5 labeled synthetic delivery system polynucleotide comprising a recognition sequence). Cells are incubated for two hours to allow for binding and uptake of the labelled DNA, then analyzed by flow cytometry. Inhibition of uptake of the labeled DNA by the unlabeled DNA is evaluated.

### Example 11: Colocalization of ctDNA and Receptors

This example demonstrates colocalization of ctDNA with receptors associated with the uptake of ctDNA.

Cells of suitable types (e.g., pancreatic cancer cells or cell lines, colorectal cancer cells or cell lines) are cultured for four hours with rhodamine-labeled ctDNA from suitable sources (e.g., pancreatic cancer, colorectal cancer, etc.), then fixed. After fixation, immunofluorescence staining is conducted with primary antibodies specific for candidate receptors (e.g., receptors identified herein, such as APLP2, PKP4, HLA-A, CD41, CD97, ITGB5, ITGA2B, Integrin αVβ5, PI4K2A, TMEM120A, PTK7A, Ceruloplasmin, PTPRF, SLC16A1, SLC7A5, ATP1B1, ATP2B4, P142A, or PIP4K2A), and an Alexa fluor 488 labelled secondary antibody for detection.

Colocalization of ctDNA with receptors is evaluated via fluorescence microscopy.

### Example 12: Reducing Uptake of ctDNA by Target Cells in a Subject

An antagonist of a ctDNA receptor identified as disclosed herein is administered (e.g., as a pharmaceutical composition) to cancer patients. The antagonist can be an antagonistic antibody or antigen-binding fragment that binds to the receptor. The antagonist can be an antagonistic antibody of, for example, PKP4, APLP2, HLA-A, or another receptor disclosed herein.

Upon administration, the uptake of ctDNA by target cells in patients is monitored via PCR of the subject's biological sample (e.g., blood, plasma, or a sample comprising cancer cells).

The effect of the antagonist on the cancer is also monitored. Response to antagonist administration on cancer progression, metastasis, and/or development of resistance to anti-cancer drugs is evaluated by suitable techniques including imaging modalities (e.g., radiography, magnetic resonance imaging, computerized tomography). Furthermore, the effect of the antagonist on the cancer is determined using criteria for cancer progression and/or tumor growth (e.g., International Uniform Response Criteria for Multiple Myeloma, RECIST criteria).

An effect of administering the antagonist on cancer progression, metastasis, and drug sensitivity is determined.

### Example 13: Evaluation of a Cancer Sample for Expression of a Receptor that Mediates Uptake of ctDNA

A biological sample (e.g., blood or cancer biopsy) is obtained from a subject with cancer (e.g., multiple myeloma, pancreatic cancer, or colon cancer) to measure expression of a ctDNA receptor disclosed herein.

Expression of a ctDNA receptor at the protein and/or mRNA levels is measured using suitable methods. For example, for detection at the protein level, immunohistochemistry is performed on a cancer biopsy with and an antibody for a ctDNA receptor. An ELISA assay (e.g., with plate coated with ctDNA receptor antibody) is performed to detect a ctDNA receptor in biological samples, such as blood and isolated plasma, from subjects with cancer and without cancer as controls. Fusion Orbitrap mass spectrometry is also performed with biological samples from subjects with cancer for unbiased proteome profiling of receptors associated with ctDNA uptake.

RNA is extracted from biological sample from subjects with cancer and subjected to qPCR and/or RNA-sequencing to measure expression of a ctDNA receptor at the mRNA level, relative to control (e.g., biological sample from a subject with no cancer).

Data are analyzed to determine whether tested biological samples express a receptor associated with ctDNA uptake at a level that is associated with the uptake of the ctDNA.

Results can be compared between samples separately assayed for uptake of ctDNA to determine thresholds of receptor expression that are associated with uptake of the ctDNA.

### Example 14: Treating Cancer on the Basis of Expression of a Receptor that Mediates Uptake of ctDNA

Biological samples from subjects with cancer are assayed for expression of a receptor associated with uptake of ctDNA as described in EXAMPLE 13. The presence/absence of receptor expression above a detectable level, or a level of expression of the receptor is used as a criterion for determining whether a subject is a candidate for administration of a therapeutic agent, such as a ctDNA-derived synthetic delivery system disclosed herein (e.g., as part of a pharmaceutical composition). For example, a subject with a cancer shown to express a receptor associated with uptake of ctDNA at a level that is associated with uptake of the ctDNA can be administered a synthetic delivery system disclosed herein that binds to and induces uptake by the receptor, while the synthetic delivery system is not administered to a subject with a cancer that does not express the receptor at a level associated with uptake of ctDNA.

1-235. (canceled)

236. A method of reducing uptake of circulating tumor-derived DNA (ctDNA) by target cells in a subject, the method comprising administering to the subject an antagonist of a receptor that is associated with cellular uptake of the ctDNA, thereby reducing the uptake of the ctDNA by the target cells in the subject.

237. The method of claim 236, wherein the antagonist comprises an antibody or an antigen-binding fragment thereof.

238. The method of claim 236, wherein the antagonist comprises an IgG antibody.

239. The method of claim 236, wherein the antagonist comprises a polypeptide.

240. The method of claim 236, wherein the antagonist comprises a small molecule antagonist of the receptor.

241. The method of claim 236, wherein the antagonist comprises DNA.

242. The method of claim 236, wherein the antagonist comprises a nucleic acid sequence identified in circulating tumor DNA (ctDNA), or a derivative thereof.

243. The method of claim 236, wherein the receptor comprises APLP2.

244. The method of claim 236, wherein the receptor comprises PKP4.

245. The method of claim 236, wherein the receptor comprises HLA-A.

246. The method of claim 236, wherein the receptor comprises CD97.

247. The method of claim 236, wherein the receptor comprises Integrin αVβ5.

248. The method of claim 236, wherein the receptor comprises PI4K2A.

249. The method of claim 236, wherein the receptor comprises PTK7A.

250. The method of claim 236, wherein the target cells comprise cancer cells.

251. The method of claim 236, wherein the target cells comprise multiple myeloma cells.

252. The method of claim 236, wherein the target cells comprise pancreatic cancer cells.

253. The method of claim 236, wherein the target cells are cancer cells and the method reduces development of resistance of the cancer cells to a therapeutic agent.

254. A method comprising determining that a biological sample from a subject expresses a receptor that is associated with uptake of ctDNA at a level that is associated with the uptake of the ctDNA.

255. A method of treating a condition in a subject in need thereof, the method comprising administering a therapeutically-effective amount of a therapeutic agent to the subject, wherein the administering is based at least in part on a result of an assay performed on a biological sample from the subject, wherein the assay determined that a receptor associated with uptake of ctDNA is expressed in the biological sample at a level that is associated with the uptake of the ctDNA
